(19) United States
(12) Patent Application Publication
(10) Pub. No.: US 2024/0066081 A1
(43) Pub. Date: Feb. 29, 2024

(54) CANINE DISTEMPER VIRUS HEMAGGLUTININ AND FUSION POLYPEPTIDES

(71) Applicant: Mayo Foundation for Medical Education and Research, Rochester, MN (US)

(72) Inventors: Stephen James Russell, Rochester, MN (US); Miguel A. Munoz Alia, Rochester, MN (US)

(21) Appl. No.: 17/767,970

(22) PCT Filed: Oct. 9, 2020
(86) PCT No.: PCT/US2020/055100

§ 371 Date: Apr. 11, 2022

Related U.S. Application Data
(60) Provisional application No. 62/913,111, filed on Oct. 9, 2019.

(51) Int. Cl. A61K 35/766; A61P 35/00; C07K 14/005; C07K 16/28; C12N 7/00

(52) U.S. Cl. CPC A61K 35/766; A61P 35/00; C07K 14/005; C07K 16/2896; C12N 7/00; C07K 2317/622; C07K 2317/92; C12N 2760/18422; C12N 2760/20232; C12N 2760/20243

(57) ABSTRACT

This document provides methods and materials related to CDV H and/or CDV F polypeptides. For example, CDV H polypeptides, CDV F polypeptides, recombinant viruses (e.g., vesicular stomatitis viruses (VSVs)) containing CDV H polypeptides and/or CDV F polypeptides, nucleic acid molecules encoding a CDV H polypeptide and/or CDV F polypeptide, methods for making recombinant viruses (e.g., VSVs) containing CDV H polypeptides and/or CDV F polypeptides, and methods for using recombinant viruses (e.g., VSVs) containing CDV H polypeptides and/or CDV F polypeptides to treat cancer or infectious diseases are provided.

### CROSS-REFERENCE TO RELATED APPLICATIONS

This application is a National Stage application under 35 U.S.C. § 371 of International Application No. PCT/US2020/055100, having an International Filing Date of Oct. 9, 2020, which claims the benefit of U.S. Provisional Application Ser. No. 62/913,111, filed Oct. 9, 2019. The disclosure of the prior application is considered part of (and is incorporated by reference in) the disclosure of this application.

### SEQUENCE LISTING

The instant application contains a Sequence Listing which has been submitted electronically in ASCII format and is hereby incorporated by reference in its entirety. Said ASCII copy, created on Nov. 25, 2020, is named 07039-1936WO1_SL.txt and is 1,096,580 bytes in size.

### 1. TECHNICAL FIELD

This document relates to canine distemper virus (CDV) hemagglutinin (H) and fusion (F) polypeptides. For example, this document relates to CDV H polypeptides, CDV F polypeptides, recombinant viruses (e.g., vesicular stomatitis viruses (VSVs)) containing CDV H polypeptides and/or CDV F polypeptides, nucleic acid molecules encoding a CDV H polypeptide and/or CDV F polypeptide, methods for making recombinant viruses (e.g., VSVs) containing CDV H polypeptides and/or CDV F polypeptides, and methods for using recombinant viruses (e.g., VSVs) containing CDV H polypeptides and/or CDV F polypeptides to treat cancer or infectious diseases.

### 2 Background Information

Viruses such as VSVs, measles viruses (MeVs), and adenoviruses can be used as oncolytic viruses to treat cancer. Vesicular stomatitis virus (VSV) is a member of the Rhabdoviridae family. The VSV genome is a single molecule of negative-sense RNA that encodes five major polypeptides: a nucleocapsid (N) polypeptide, a phosphoprotein (P) polypeptide, a matrix (M) polypeptide, a glycoprotein (G) polypeptide, and a viral polymerase (L) polypeptide.

### SUMMARY

This document provides methods and materials related to CDV H and/or CDV F polypeptides. For example, this document provides CDV H polypeptides, CDV F polypeptides, recombinant viruses (e.g., vesicular stomatitis viruses (VSVs)) containing CDV H polypeptides and/or CDV F polypeptides, nucleic acid molecules encoding a CDV H polypeptide and/or CDV F polypeptide, methods for making recombinant viruses (e.g., VSVs) containing CDV H polypeptides and/or CDV F polypeptides, and methods for using recombinant viruses (e.g., VSVs) containing CDV H polypeptides and/or CDV F polypeptides to treat cancer or infectious diseases.

As described herein, CDV F polypeptides can be designed to have increased fusogenic activity when expressed by cells in combination with a CDV H polypeptide as compared to the level of fusogenic activity of a wild-type CDV F polypeptide expressed by comparable cells in combination with that CDV H polypeptide. For example, CDV F polypeptides designed to have a truncated signal peptide sequence can exhibit increased fusogenic activity when expressed by cells in combination with a CDV H polypeptide (e.g., a wild-type or de-targeted CDV H polypeptide) as compared to the level of fusogenic activity of a wild-type CDV F polypeptide containing a full length signal peptide sequence expressed by comparable cells in combination with that CDV H polypeptide. Such CDV F polypeptides can be incorporated into a virus to create a recombinant virus having the ability to increase fusogenic activity observed in cells infected by that virus.

As also described herein, CDV H polypeptides can be designed to be de-targeted such that they do not have the ability, when used in combination with an F polypeptide (e.g., a CDV F polypeptide), to enter cells via, or fuse cells via, a Nectin 4 polypeptide, a SLAMF1 polypeptide, or a virus receptor present on wild-type Vero cells. Such CDV H polypeptides can provide a platform for designing H polypeptides having the ability to be re-targeted to one or more targets of interest. For example, an H polypeptide provided herein can be further engineered to contain a binding sequence (e.g., a single chain antibody (scFv) sequence) having binding specificity for a target of interest such that a recombinant virus containing that re-targeted H polypeptide, and an F polypeptide, can infect cells expressing that target.

In addition, viruses such as VSVs can be designed to have a nucleic acid molecule that encodes a VSV N polypeptide, a VSV P polypeptide, a VSV M polypeptide, a CDV F polypeptide (e.g., a wild-type CDV F polypeptide or an engineered CDV F polypeptide described herein), a CDV H polypeptide (e.g., a wild-type CDV H polypeptide or an engineered CDV H polypeptide described herein), and a VSV L polypeptide. Such a nucleic acid molecule can lack a functional VSV G polypeptide and/or lack the nucleic acid sequence that encodes a full-length VSV G polypeptide. For example, a VSV provided herein can be designed to have a nucleic acid molecule that encodes a VSV N polypeptide, a VSV P polypeptide, a VSV M polypeptide, a CDV F polypeptide (e.g., a wild-type CDV F polypeptide or an engineered CDV F polypeptide described herein), a CDV H polypeptide (e.g., a wild-type CDV H polypeptide or an engineered CDV H polypeptide described herein), and a VSV L polypeptide and lacks the ability to encode a functional VSV G polypeptide. In some cases, a VSV provided herein can be designed to have a nucleic acid molecule that encodes a VSV N polypeptide, a VSV P polypeptide, a VSV M polypeptide, a CDV F polypeptide (e.g., a wild-type CDV F polypeptide or an engineered CDV F polypeptide described herein), a CDV H polypeptide (e.g., a wild-type CDV H polypeptide or an engineered CDV H polypeptide described herein), and a VSV L polypeptide with the nucleic acid sequence encoding the CDV F polypeptide and the CDV H polypeptide being located in the position where the nucleic acid sequence encoding a full-length VSV G polypeptide is normally located in a wild-type VSV. In some cases, a VSV provided herein can be designed to have a nucleic acid molecule where the nucleic acid sequence encoding a VSV G polypeptide is replaced with nucleic acid that encodes a CDV F polypeptide (e.g., a wild-type CDV F polypeptide or an engineered CDV F polypeptide described herein) and a CDV H polypeptide (e.g., a wild-type CDV H polypeptide or an engineered CDV H polypeptide described herein).

As described herein, VSV/CDV hybrids can be designed to have CDV selectivity and a rapid replication as observed with wild-type or parental VSV. In some cases, a VSV/CDV hybrid provided herein can be designed to have a preselected tropism. For example, CDV F and/or H polypeptides having knocked out specificity for Nectin-4 and/or SLAMF1 can be used. In such cases, a scFv or polypeptide ligand can be attached to, for example, the C-terminus of the CDV H polypeptide. In such cases, the scFv or polypeptide ligand can determine the tropism of the VSV/CDV hybrid. Examples of scFvs that can be used to direct VSV/CDV hybrids to cellular receptors (e.g., tumor associated cellular receptors) include, without limitation, anti-EGFR, anti-αFR, anti-CD46, anti-CD38, anti-HER2/neu, anti-EpCAM, anti-CEA, anti-CD20, anti-CD133, anti-CD117 (c-kit), and anti-CD138 and anti-PSMA scFvs. Examples of polypeptide ligands that can be used to direct VSV/CDV hybrids include, without limitation, urokinase plasminogen activator uPA polypeptides, cytokines such as IL-13 or IL-6, single chain T cell receptors (scTCRs), echistatin polypeptides, stem cell factor (SCF), EGF and integrin binding polypeptides.

In some cases, a VSV/CDV hybrid provided herein can have a nucleic acid molecule that includes a sequence encoding an interferon (IFN) polypeptide (e.g., a human IFN-β polypeptide), a sodium iodide symporter (NIS) polypeptide (e.g., a human NIS polypeptide), a fluorescent polypeptide (e.g., a GFP polypeptide), any appropriate therapeutic transgene (e.g., HSV-TK or cytosine deaminase), polypeptide that antagonizes host immunity (e.g., influenza NS1, HSVγ34.5, or SOCS1), or tumor antigen (e.g., cancer vaccine components). The nucleic acid encoding the IFN polypeptide can be positioned between the nucleic acid encoding the VSV M polypeptide and the nucleic acid encoding the VSV L polypeptide. Such a position can allow the viruses to express an amount of the IFN polypeptide that is effective to activate anti-viral innate immune responses in non-cancerous tissues, and thus alleviate potential viral toxicity, without impeding efficient viral replication in cancer cells. The nucleic acid encoding the NIS polypeptide can be positioned between the nucleic acid encoding the VSV M polypeptide and the VSV L polypeptide. Such a position of can allow the viruses to express an amount of the NIS polypeptide that (a) is effective to allow selective accumulation of iodide in infected cells, thereby allowing both imaging of viral distribution using radioisotopes and radiotherapy targeted to infected cancer cells, and (b) is not so high as to be toxic to infected cells. Positioning the nucleic acid encoding an IFN polypeptide between the nucleic acid encoding the VSV M polypeptide and the nucleic acid encoding the VSV L polypeptide and positioning the nucleic acid encoding a NIS polypeptide between the nucleic acid encoding the VSV M polypeptide and the VSV L polypeptide within the genome of a VSV can result in VSVs that are viable, that have the ability to replicate and spread, that express appropriate levels of functional IFN polypeptides, and that expression appropriate levels of functional NIS polypeptides to take up radio-iodine for both imaging and radio-virotherapy.

In general, one aspect of this document features a CDV F polypeptide having signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide.

In another embodiment, this document features a nucleic acid molecule encoding a CDV F polypeptide. The CDV F polypeptide can have a signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide.

In another embodiment, this document features a recombinant virus comprising a CDV F polypeptide. The CDV F polypeptide can have a signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide.

In another embodiment, this document features a recombinant virus comprising a nucleic acid molecule. The nucleic acid molecule can encode a CDV F polypeptide. The CDV F polypeptide can have a signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide.

In another embodiment, this document features a CDV H polypeptide comprising 454A, 464A, 479A, 494A, 510A, 520A, 525A, 526A, 527S, 528A, 529A, 537A, 539A, 547A, 548A, or a combination thereof according to the amino acid numbering of SEQ ID NO:2. The CDV H polypeptide can comprise a combination of two, three, four, five, or six of 454A, 464A, 479A, 494A, 510A, 520A, 525A, 526A, 527S, 528A, 529A, 537A, 539A, 547A, and 548A. The CDV H polypeptide can comprise a combination of seven, eight, nine, ten, or eleven of 454A, 464A, 479A, 494A, 510A, 520A, 525A, 526A, 527S, 528A, 529A, 537A, 539A, 547A, and 548A. The CDV H polypeptide can comprise a combination of 12, 13, or 14 of 454A, 464A, 479A, 494A, 510A, 520A, 525A, 526A, 527S, 528A, 529A, 537A, 539A, 547A, and 548A. The CDV H polypeptide can comprise 454A, 464A, 479A, 494A, 510A, 520A, 525A, 526A, 527S, 528A, 529A, 537A, 539A, 547A, and 548A. The CDV H polypeptide can comprise M437 according to the amino acid numbering of SEQ ID NO:5.

In another embodiment, this document features a CDV H polypeptide comprising the sequence set forth in FIG. 11 except that the sequence comprises a mutation of a presented amino acid residue selected from the group consisting of P454, V/L/F460, L/F/W479, I494, I/J/V510, Y520, Y/N525, D/G526, I/V527, S/T528, R529, Y/D537, Y539, Y/F547, and T/M548 according to the amino acid numbering of SEQ ID NO:5. The CDV H polypeptide can comprise a mutation of two, three, four, five, or six presented amino acid residues selected from the group. The CDV H polypeptide can comprise a mutation of seven, eight, nine, ten, or eleven presented amino acid residues selected from the group. The CDV H polypeptide can comprise a mutation of 12, 13, or 14 presented amino acid residues selected from the group. The CDV H polypeptide can comprise a mutation of the presented amino acid residues of the group. The CDV H polypeptide can comprise M437 according to the amino acid numbering of SEQ ID NO:5.

In another embodiment, this document features a nucleic acid molecule encoding a CDV H polypeptide. The CDV H polypeptide can comprise the sequence set forth in FIG. 11 except that the sequence comprises a mutation of a presented amino acid residue selected from the group consisting of P454, V/L/F460, L/F/W479, I494, I/L/V510, Y520, Y/N525, D/G526, I/V527, S/T528, R529, Y/D537, Y539, Y/F547, and T/M548 according to the amino acid numbering of SEQ ID NO:5. The CDV H polypeptide can comprise a mutation of two, three, four, five, or six presented amino acid residues selected from the group. The CDV H polypeptide can comprise a mutation of seven, eight, nine, ten, or eleven presented amino acid residues selected from the group. The CDV H polypeptide can comprise a mutation of 12, 13, or 14 presented amino acid residues selected from the group. The CDV H polypeptide can comprise a mutation of the presented amino acid residues of the group. The CDV H polypeptide can comprise M437 according to the amino acid numbering of SEQ ID NO:5.

In another embodiment, this document features a recombinant virus comprising a CDV H polypeptide. The CDV H polypeptide comprising the sequence set forth in FIG. 11 except that the sequence comprises a mutation of a presented amino acid residue selected from the group consisting of P454, V/L/F460, L/F/W479, I494, I/L/V510, Y520, Y/N525, D/G526, I/V527, S/T528, R529, Y/D537, Y539, Y/F547, and T/M548 according to the amino acid numbering of SEQ ID NO:5. The CDV H polypeptide can comprise a mutation of two, three, four, five, or six presented amino acid residues selected from the group. The CDV H polypeptide can comprise a mutation of seven, eight, nine, ten, or eleven presented amino acid residues selected from the group. The CDV H polypeptide can comprise a mutation of 12, 13, or 14 presented amino acid residues selected from the group. The CDV H polypeptide can comprise a mutation of the presented amino acid residues of the group. The CDV H polypeptide can comprise M437 according to the amino acid numbering of SEQ ID NO:5. The virus can comprise a CDV F polypeptide, wherein the CDV F polypeptide comprises a signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide.

In another embodiment, this document features a recombinant virus comprising a nucleic acid molecule encoding a CDV H polypeptide. The CDV H polypeptide can comprise the sequence set forth in FIG. 11 except that the sequence comprises a mutation of a presented amino acid residue selected from the group consisting of P454, V/L/F460, L/F/W479, I494, I/L/N510, Y520, Y/N525, D/G526, I/V527, S/T528, R529, Y/D537, Y539, Y/F547, and T/M548 according to the amino acid numbering of SEQ ID NO:5. The CDV H polypeptide can comprise a mutation of two, three, four, five, or six presented amino acid residues selected from the group. The CDV H polypeptide can comprise a mutation of seven, eight, nine, ten, or eleven presented amino acid residues selected from the group. The CDV H polypeptide can comprise a mutation of 12, 13, or 14 presented amino acid residues selected from the group. The CDV H polypeptide can comprise a mutation of the presented amino acid residues of the group. The CDV H polypeptide can comprise M437 according to the amino acid numbering of SEQ ID NO:5.

The virus can comprise a nucleic acid molecule encoding a CDV F polypeptide. The CDV F polypeptide can have a signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide.

In another embodiment, this document features a recombinant virus described herein that is a hybrid virus of (a) CDV and (b) VSV, MeV, or Adenovirus.

In another embodiment, this document features a replication-competent vesicular stomatitis virus comprising an RNA molecule, wherein the RNA molecule comprises a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a CDV F polypeptide, a nucleic acid sequence that is a template for a positive sense transcript encoding a CDV H polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide. The CDV F polypeptide can have a signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide. The CDV H polypeptide can be a CDV H polypeptide described in one of the preceding paragraphs. The CDV H polypeptide can comprises an amino acid sequence of a single chain antibody. The single chain antibody can be a single chain antibody directed to CD19, CD20, CD38, CD46, EGFR, αFR, HER2/neu, or PSMA. The RNA molecule can comprise a nucleic acid sequence that is a template for a positive sense transcript encoding a NIS polypeptide.

In another embodiment, this document features a composition comprising a virus of any of the preceding paragraphs.

In another embodiment, this document features a nucleic acid molecule comprising a nucleic acid strand comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a CDV F polypeptide, a nucleic acid sequence that is a template for a positive sense transcript encoding a CDV H polypeptide, and a nucleic acid sequence that is a template for a positive sense transcript encoding a VSV L polypeptide, wherein the nucleic acid strand lacks a nucleic acid sequence that is a template for a positive sense transcript encoding a functional VSV G polypeptide. The CDV F polypeptide can have a signal peptide sequence that is less than 75 amino acid residues in length. The signal peptide sequence can comprise no more than 75 amino acid residues of SEQ ID NO:6. The CDV F polypeptide can comprise SEQ ID NO:4 with the proviso that the CDV F polypeptide lacks at least amino acid residues 1 to 60 or lacks at least amino acid residues 1 to 105 of SEQ ID NO:4. A recombinant virus comprising the CDV F polypeptide and a CDV H polypeptide can exhibit increased fusogenic activity as compared to a comparable control recombinant virus comprising a full length, wild-type CDV F polypeptide and the CDV H polypeptide. The CDV H polypeptide can be a CDV H polypeptide described in one of the preceding paragraphs. The CDV H polypeptide can comprises an amino acid sequence of a single chain antibody. The single chain antibody can be a single chain antibody directed to CD19, CD20, CD38, CD46, EGFR, αFR, HER2/neu, or PSMA. The RNA molecule can comprise a nucleic acid sequence that is a template for a positive sense transcript encoding a NIS polypeptide.

In another embodiment, this document features a composition comprising a nucleic acid molecule of any of the preceding paragraphs.

In another embodiment, this document features a method for treating cancer. The method comprises administering a composition described herein (e.g., a composition containing a virus described herein) to a mammal comprising cancer cells, wherein the number of cancer cells within the mammal is reduced following the administration. The mammal can be a human. The cancer can be myeloma, melanoma, glioma, lymphoma, mesothelioma, lung cancer, brain cancer, stomach cancer, colon cancer, rectum cancer, kidney cancer, prostate cancer, ovary cancer, breast cancer, pancreas cancer, liver cancer, or head and neck cancer.

In another embodiment, this document features a method for inducing tumor regression in a mammal. The method comprises administering a composition described herein (e.g., a composition containing a virus described herein) to a mammal comprising a tumor, wherein the size of the tumor is reduced following the administration. The mammal can be a human. The cancer can be myeloma, melanoma, glioma, lymphoma, mesothelioma, lung cancer, brain cancer, stomach cancer, colon cancer, rectum cancer, kidney cancer, prostate cancer, ovary cancer, breast cancer, pancreas cancer, liver cancer, or head and neck cancer.

In another embodiment, this document features a method for rescuing replication-competent vesicular stomatitis viruses from cells. The vesicular stomatitis viruses comprise an RNA molecule comprising a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a CDV F polypeptide, a nucleic acid sequence that is a template for a positive sense transcript encoding a CDV H polypeptide, and a nucleic acid sequence that is a template for a positive sense transcript encoding a VSV L polypeptide, wherein the RNA molecule lacks a nucleic acid sequence that is a template for a positive sense transcript encoding a functional VSV G polypeptide. The method comprises (a) inserting nucleic acid encoding the RNA molecule into the cells under conditions wherein replication-competent vesicular stomatitis viruses are produced, and (b) harvesting the replication-competent vesicular stomatitis viruses.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Molecular phylogenetic analysis by Maximum Likelihood method of the hemagglutinin gene of CDV genotypes. The tree was inferred by using the Maximum Likelihood method based on the General Time Reversible model. The analysis involved 119 complete CDV H nucleotides sequences. The strains 22458/15 and 5804 were classified as Artic-like and Europe-1/South America 1 genotypes, respectively. Evolutionary analyses were conducted in MEGA7.

FIG. 2. Generation CDV H/F complexes for targeted-cell fusion. (A) Syncytia formation assay: Monolayers of Vero cells and Vero cells expressing human Nectin-4 polypeptides or dog SLAMF1 polypeptides were co-transfected with H and F polypeptide-encoding expression plasmids from CDV₅₈₀₄, CDV_{22458/16}, or CDV_{onderstepoort} (large-plaque-forming) (CDV_{OL}) as indicated. Syncytia formation was recorded 24 hours later after Giemsa staining. (B) Cells were additionally transfected with a GFP expression plasmid to enhance the sensitivity. DAPI was used for nuclei staining. (C) Schematic drawing of a receptor expanded morbillivirus attachment protein. The receptor binding protein contained a cytoplasmic tail (C), transmembrane domain (T), and an ectodomain fused to a CD38-specific single chain variable fragment (scFv) followed by a His tag. (D) Surface expression of receptor expanded morbillivirus attachment proteins. HEK293T cells were transfected with the indicated attachment proteins retargeted against CD38 receptor, and surface expression was analyzed by FACS using a PE-conjugated anti-HIS antibody. (E) Schematic representation of the quantitative fusion assay based on the self-associating split luciferase assay. Effector cells were transfected with H and F polypeptide-encoding plasmids and plasmids encoding half of the renilla luciferase (RL) and green fluorescence proteins (GFP). Target cells, bearing the receptors, were transfected with plasmids encoding the other half of dual-split reporter genes and mixed with the effector cells. Upon content mixing, the otherwise nonfunctional halves of the reporter genes reconstitute the enzyme activity, which was measured in real time. (F) Cell-targeted fusion activity of CD38-receptor expanded morbillivirus attachment proteins. Effector cells were co-transfected with the attachment/fusion polypeptide pairs indicated. After co-culture with the target cells, luminescence signal was registered. Values and error bars (SDs) were from one representative experiment performed at least in triplicate. Of note, the CDV F signal peptide was replaced with the homologous form from MEV (MeV SP). Statistical significance was calculated by two-way ANOVA with Turkey multiple-comparison test. *, p<0.02; ***, p<0.0005; ****, p<0.0001. (G) Integrity of CDV H polypeptides. HEK293T cells were transfected with the indicated CDV H polypeptides, and cell lysates were immunoblotted with anti-HIS antibodies (CDV H) or anti-β-actin (loading control). (H) Cell-targeted fusion of retargeted H/F complexes. Cell fusion was determined as described in (C). Effector cells were co-transfected with the receptor-blind, CD38 retargeted pairs MeV H/F and CDV H₅₈₀₄/F_{22458/16} and overlaid with CHO cells expressing relevant receptors. Values and error bars (SDs) were from one representative experiment performed at least in triplicate. (I) Retargeting the CDV H/F complex to Her2/neu. A cell fusion assay was performed using CDV H/F complexes displaying Her2/neu-specific scFv or affibodies (Z_{X}). Binding affinities for the displayed ligand are shown. (J) Relationship between receptor expression and receptor-binding affinity. Fusion activity of CDV H polypeptides displaying Her2/neu-specific affibodies with different affinities was assessed on Her2/neu positive cell lines: HT1080 (3.4×10³ molecules/cell), Sko3pi (4.19×10³ molecules/cell), TET67L (1.5×10⁵ molecules/cell). Cells transfected only with CDV F were used as a negative control.

FIG. 3. H/F complexes from CDV can replace the envelope glycoproteins from measles virus to result in cell-targeted entry and anti-measles virus antibody resistance. (A) Cell surface expression. CHO cells were transfected with the indicated H polypeptide expressing plasmid (HIS-tagged) along with one of the DSP plasmids and a F polypeptide (MeV or CDV F224568/16) expressing plasmid. 24 hours later, H expression was determined by CELISA with anti-HIS antibodies. (B) CHO cells transfected as in (A) were co-cultured with the indicated CHO cell derivate and luminescence values were obtained for a 9-hour time course. Values and error bars (SDs) were from one representative experiment performed at least in triplicate. (C) Protein composition of measles virus encoding CDV H/F complexes targeted against CD46 (scFv A09, Stealth 2.0). 1.6E4 TCID₅₀ particles were subjected to SDS-PAGE and immunoblotted with the relevant antibody. Measles Virus was used as a control. (D) Replication kinetics. The growth kinetic of Stealth 2.0 virus was determined on Vero and Vero-HIS (multiplicity of infection (MOI)=0.03) at the indicated time points. The growth kinetics of MeV on Vero/hSLAMF1 cells was included for comparison purposes. (E) Neutralization assay. A fluorescence focus reduction neutralization assay was performed with MeV and CDV antisera. Different dilutions of the antiserum were pre-incubated for 1 hour at 37° C. with a fixed amount of virus. The mix was then used to infect Vero-HIS cells, and the amount of virus in control wells without antibodies was set to 100%. All neutralization curves represent the mean and SD of four replicate curves run on the same 96-well plate. (F) Receptor-selectivity of recombinant viruses. CHO cells expressing the relevant receptors were infected with the recombinant viruses at the indicated MOI. GFP auto-fluorescence was recorded 2 days later.

FIG. 4. Oncolytic activity of CD46-targeted envelope chimeric MeV. CDB17 SCID mice were implanted subcutaneously with the human myeloma cell line U266.B1. When tumor volume reached 500 mm³, mice were randomized and left untreated (PBS control) or treated once with 1×10⁵ TCID⁵⁰ particles administered intravenously. Tumor volumes (B) and survival (C) was recorded afterwards.

FIG. 5. Schematic of exemplary recombinant VSVs according to some embodiments. VSV-hIFNβ-NIS: VSV Indiana was engineered to express human interferon beta (hIFNβ) at the M/G intergenic region, and human sodium iodide symporter (NIS) at the G/L intergenic region and rescued as described elsewhere (Naik et al., Leukemia, 26:1870-78 (2012)). VSV expressing CDV-F_{22455/16} (with the CDV F polypeptide signal peptide replaced with that of MeV F polypeptide) and CDV-H₅₈₀₄ were generated using the pVSV-smart platform. Point mutations in the CDV-H₅₈₀₄ polypeptide, Y539A and R529A, were introduced by site-directed mutagenesis to ablate natural tropisms to canine Nectin-4 and SLAMF1 polypeptides, respectively. Targeted viruses were generated by displaying EGFR or CD38-targeted scFvs at the C-terminus of CDV-H₅₈₀₄ with an IGES linker peptide and an H6 polyhistidine tag (SEQ ID NO: 21). Retargeted VSV-CDV F/H constructs were rescued on Vero-anti-H6, allowing infection, virus amplification, and fusion of target cells. The titers for each recombinant virus are indicated.

FIG. 6. Monolayers of the indicated CHO cells (wild-type or stably overexpressing the indicated receptors) were mock infected or infected (MOI=0.2) with VSV-CDVFH-GFP or VSV-CDVF/Hₐₐ-αEGFR-GFP. Fluorescent micrographs were taken under 100× magnification at the indicated times. GFP expression (green) correlates with virus infection and spread within the monolayers.

FIG. 7. Monolayers of the indicated CHO cells (wild-type or stably expressing receptors EGFR or CD38) were infected (MOI=0.1) with VSV-CDVFHₐₐ-αEGFR or VSV-CDVFHₐₐ-αCD38. After 42 hours, cell monolayers were fixed with paraformaldehyde and stained with crystal violet. Photos were taken under 40× magnification.

FIG. 8. Therapeutic effects of chimeric VSV-CDVFHaa-αEGFR against xenografted human ovarian cancer in the peritoneum. Female 5-6 week-old athymic nude mice (Envigo, Indianapolis, IN) were implanted intraperitoneally with 2×10⁶ SKOV3ip.1-Fluc cells (200 μL/mouse) (day −7). 7 days post-implantation (day 0), tumor-bearing mice were randomized based on firefly luciferase signals using IVIS spectrum (Perkin Elmer, Hopkinton, MA). Mice were identified by microchip and ear notch. Following randomization, mice received a single dose of 1×10⁷ TCID₅₀ of virus or saline control (250 μL/mouse) via intraperitoneal injection. Mice were euthanized if they developed ascites, subcutaneous injection site tumors that were >10% of body weight, or if they lost >20% of body weight. All surviving mice were euthanized at the end of the experiment (day 92 after virus treatment). Kaplan-Meier survival curves were plotted and compared by log-rank sum test. Clinical observations and weight were recorded three times per week until end of study or euthanasia of the mouse.

FIG. 9 is a nucleic acid sequence (SEQ ID NO:1) of a CDV H open reading frame encoding a CDV H polypeptide (SEQ ID NO:2).

FIG. 10 is a nucleic acid sequence (SEQ ID NO:3) of a CDV F open reading frame encoding a CDV F polypeptide (SEQ ID NO:4).

FIG. 11. Retargeting wild-type CDV envelope to EGFR and CD38. (A) Schematic representation of cloning strategy to generate retargeted wild-type CDV H polypeptides (top). Standard one-letter amino acid abbreviations are used to denote the changes introduced to ablate native receptor (SLAMF1 and Nectin-4) usage (bottom). The amino acid numbering is with respect to SEQ ID NO:5. The single-chain antibody fragments are displayed as a C-terminal extension of the H glycoprotein using a Factor Xa (Fxa) cleavage site (an IEGR amino acid sequence). There is an optional six-histidine tag (SEQ ID NO: 21) in all of the constructs to facilitate virus rescue on Vero-His cells. (B) Co-transfection experiments demonstrating targeted fusion competence of receptor blind H polypeptide targeted to CD38. CHO-CD38 cells in 12 well plates were co-transfected with either CMV driven CDV F plasmid and CMV driven wild-type CDV H-CD38 or CMV driven receptor blind CDV H-CD38, and cells were fixed, stained and imaged 24 hours later. (C) Targeted cell fusion mediated by CDV H constructs was resistant to pooled measles immune human serum. CHO-CD38 cells were co-transfected with CMV driven H and F plasmids together with a CMV driven GFP plasmid for visualization and incubated with indicated dilutions. Cells were photographed 24 hours after transfection. (D) Chimeric measles viruses bearing targeted CDV H polypeptides retain specificity on a panel of CHO cells expressing the desired receptors or human tumor cell lines with the desired receptor. Cell lines were infected with the respective viruses at an MOI of 0.5, and photographed were taken 48 hours later. (E) Schematic design of experiment to test in vivo oncolytic efficacy and specificity of chimeric measles viruses with retargeted CDV envelope. Nude mice were implanted with 5×10⁶ SKOV3ip-fluc cells either subcutaneously (SQ) or intraperitoneally (IP) followed by 6 doses of 1×10⁶ TCID₅₀/mL intratumorally (IT) for subcutaneous tumors or 2×10⁶ TCID₅₀/mL IP for IP tumors every other day beginning on day 10. (F) Individual tumor volumes of subcutaneous SKOV3ip tumors treated with respective viruses (top) and survival of animals with intraperitoneal tumors treated with respective viruses (bottom).

FIG. 12 is an alignment of a representative number of CDV H polypeptides. The top sequence (designated AF378705.1_America1) is SEQ ID NO:5 and used for numbering purposes where indicated. FIG. 12 discloses SEQ ID NOS 5, 31-109, 105, 110-145, and 227-228, respectively, in order of appearance.

FIG. 13 is an alignment of a representative number of CDV F polypeptides. The signal peptide sequence extends from amino acid position 1 to amino acid position 135. The top sequence (designated AF378705.1_America1) is SEQ ID NO:7 and used for numbering purposes where indicated. FIG. 13 discloses SEQ ID NOS 7 and 146-226, respectively, in order of appearance.

FIG. 14. CDV OL can infect cells lacking SLAMF1 and NECTIN4 receptors. (A) Assessment of infection of Vero cells by CDV isolates in comparison with the OL strain. Cells were infected at an MOI of 0.1 (determined on Vero-dogSLAMF1 cells) and stained with Hema-Quick 48 hours later for visualization. (B) A panel of CHO cells expressing different relevant receptors was infected with an eGFP reporter MeV comprising the CDV H/F OL glycoproteins. Infectivity was reported using a fluorescence microscope. Magnification 40×.

FIG. 15. Heterologous combination of wild-type CDV H/F with a shortened signal peptide results in enhanced receptor-dependent fusion due to a weaker H-F interaction. (A) Syncytia formation in cells cotransfected with CDV-F, CDV-H, and eGFP. The signal peptides of CDV-F were replaced with the homolog from MeV-F, as indicated by the black boxes in the schematic. Twenty-four hours after cotransfection, fusion score was assessed under the GFP channel. (B) Quantitative fusion assay. Effector BHK cells were transfected with the indicated combination of attachment (CDV-H or Nipah-G) and fusion (F) proteins plus one of the dual-split reporter plasmids. Target CHO cells and CHO cells expressing CD38 (CHO-CD38) were transfected with the other dual-split reporter plasmids. 16 hours post-transfection cells were overlaid and Renilla luciferase activity was determined (RLU) 8 hours later. Values represent the mean ±standard deviation (SD) of one representative experiment performed in triplicate. Statistical significance was determined using one-way ANOVA with Holm-Sidak's multiple comparison test (ns, not significant *, p<0.05; **, p<0.002; ***, p<0.0001). (C) CDV-H/F coimmunoprecipitation. HEK293T cells transiently expressing either wt or mutant HIS-tagged CDV-H proteins together with FLAG-tagged CDV-F proteins were lysed and immunoprecipitated (IP) with an anti-FLAG antibody. The signal intensity was determined using an anti-HIS antibody. (D) Quantitative fusion assay of fully retargeted CDV-H and MeV-H proteins onto CHO cells and CHO cells derivates. Either HIS-tagged or HIS-tagged and CD38-retargeted MeV-H/F and CDV-H/F complexes were transfected into effector cells and luminescence signal was determined over time. MeV-Haals=MeV-H blinded for CD46, NECTIN-4 and SLAMF1 via mutations Y481A, R533A, S548L and F549S.

FIG. 16. Conservation of amino acid residue M437 in the CDV-H protein among different genetic groups. Sequence alignment was performed with CDV-H sequences retrieved from GenBank, including the CDV-H sequence determined here for the SPA.Madrid/22458/16 isolate. The accession numbers are indicated.

FIG. 17. Integrity and expression of chimeric ligand-displaying receptor binding proteins. (A) Western blot analysis of HEK293T cells transfected with the indicated proteins fused to an anti-CD38 scFv or not. Proteins were blotted with an anti-HIS antibody or an anti-β-actin antibody (loading control). (B) Protein expression of the attachment proteins and mutants on HEK293T cells fixed with or without permeabilization analyzed by flow cytometry. Histograms are from one representative experiment out of two biological replicates. Geometric mean intensity f SD from two biological replicates is shown at the upper right comer of each histogram. Filled curves denote cells transfected with empty plasmids.

FIG. 18. FLAG tag insertion in the F ectodomain and its effect on protein bioreactivity. (A) Schematic drawings of uncleaved MeV-F and CDV-F. The NH₂ and COOH termini, signal peptide (SP), fusion peptide (FP), and transmembrane (TM) and cytoplasmic regions are indicated. The sequence surrounding the cleavage site (in bold) and that of the fusion peptide are shown. The numbering considers the homotypic signal peptides. (B) Syncytia formation in Vero cells after cotransfection of homologous H and F expression plasmids with FLAG insertions at different positions. Cells were stained at 16 hours post transfection, and microphotographs were acquired for quantification. (C) Quantification of syncytia formation. The data are shown as the mean±SD (n=20). Significance was determined using one-way ANOVA with Holm-Sidak's multiple comparison test (ns, not significant; ***, p<0.001). (D) Dual-split protein fusion assay for the cotransfection of CDV-H/F SPA with or without a FLAG-tag insertion at amino acid 216. The luciferase signal was measured at 8 hours. The experiment was performed in technical duplicates.

FIG. 19. CD46 specificity of scFvs. (A) SDS-PAGE analysis of target proteins. MW: molecular weight ladder, C: Coomassie staining; WB: western blot analysis using an anti-CD46 antibody. (B) Size exclusion chromatography trace for the CD46 used in the experiments. The estimated MW is from a calibration curve is indicated. (C) Binding of scFv-Fc tagged fusion proteins to CD46 or NECTIN4 as determined by ELISA. Detection was performed with the Fc portion used as a control for the amount of protein. Experiments were performed in technical duplicates. The data are shown as the mean ±SD n=2). Significance was determined using one-way ANOVA with Holm-Sidak's multiple comparison test. *, p<0.05; **, p<0.005.

FIG. 20. CD46 binding affinity of the displayed scFv determines CD46-dependent cell-to-cell fusion of the retargeted CDV H/F complex. (A) Representative sensogram (in resonance units, RU) for the binding of CD46 to biosensor surfaces containing (continuous line) or lacking (discontinuous line) single-chain antibody fragments (scFv). Experimental data represents the injection for 300s of scFv K2 followed by buffer injection. 1 μM of CD46 was subsequently flown over both biosensor surfaces and the signal was recorded during (association) and after injection (dissociation). The surfaces were finally regenerated at the end of the cycle as described herein. (B) Binding of CD46 to scFvs as assessed by surface plasmon resonance. Sensograms showing the response units (black lines) of various concentrations of CD46 to the scFvs. Best fit 1:1 binding model is shown as discontinuous red lines. Binding affinity (Kd) was determined from the association and dissociation rates (Table 1). (C) Quantitative fusion assay for the MeV-H and CDV-H variants on CHO cells. The experiment was carried out in duplicate and repeated twice with similar results (see FIG. 21). The data are shown as the mean f SD.

FIG. 21. Binding affinity of the scFv displayed onto the CDV-H/F complex drives enhanced cell-cell fusion. (A) Cellular enzyme-linked immunosorbent assay (CELISA) for the amount of cellular protein used in the quantitative fusion assay on FIG. 20C. A CELISA was performed on CHO cells transfected with the indicated attachment protein using an anti-6×HIS-tag monoclonal antibody (n=5). (B) Quantitative fusion assay for the CD46-retargeted CDV-H/F complex using affinity tuned scFvs (same data as presented in FIG. 20C). Y539A indicates the substitution in CDV-H to ablate the natural tropism for NECTIN4.

FIG. 22. CD46-retargeted CDV envelope glycoproteins determine virus tropism. (A) Schematic representation of Stealth: a vaccine-derived measles virus pseudotyped with CD46-retargeted CDV H/F envelope proteins. Created with BioRender.com. (B) Role of the CD46 binding affinity into virus entry. Cells were infected at the indicated MOI with Stealth viruses displaying scFv with different affinity to CD46, eGFP expression was monitored 48 h post infection. (C) CHO cells and derivates expressing the HIS-pseudoreceptor or CD46 were infected with Fluc-expressing Stealth viruses (K1 and A09) at MOI 0.5. Luciferase expression was measured 48 hours postinfection. n=2 except for CHO-CD46, *, p-value<0.05 (two-tailed t-test), (D) Multistep growth kinetics of Stealth-A09 in Vero or Vero-αHIS cells. At the indicated time-point, both the supernatant and the cell pellet were collected and virus titers were determined on Vero-αHIS cells. Values and error bars (SD) were determined for a representative experiment performed in triplicate. (E) Protein composition of viruses. Western blot analysis was determined with similar amounts of virus particles and probed with the relevant antibodies. The molecular weight of the standard is indicated. (F) Virus tropism. CHO cell derivatives were infected with the eGFP-expressing viruses as indicated. eGFP autofluorescence was determined 48 hours later. Scale bar, 200 pm. (G) Genetic stability of Stealth. Vero-hSLAMF1 cells were infected with Stealth and passaged multiple times. After 8 passages, the recovered virus was used to infect Vero cells expressing human or canine SLAMF1. Representative microphotographs are shown after infection for three or six days.

FIG. 23. Assessment of receptor interactions for the engineered CDV fusion apparatus complex. Cells were cotransfected with MeV-F and MeV-H or CDV-F and CDV-H retargeted variants with a CD46-specific scFv. For visualization purposes, an expression plasmid encoding eGFP was cotransfected, and eGFP autofluorescence was visualized at 24 hours post transfection. Y539A indicates the substitution in CDV-H to ablate the natural tropism for NECTIN4. “+” and “−” symbols were used for semiquantification (same as presented in FIG. 15A). “Undisplay” indicates no scFv.

FIG. 24. High CD46 binding affinity governs oncolytic activity of CD46-targeted Stealth virus in a mouse model of ovarian cancer. (A) Study schematic of the experimental design. SKVOv3ip.1 tumor cells encoding the firefly luciferase gene (SKOV3ip.Fluc) were implanted intraperitoneally into athymic mice. At day 10, 1×10⁶ TCID₅₀ particles of Stealth were administered following the same route. Tumor burden was then monitored at 7 days interval through bioluminescence imaging (BLI). (B) Kaplan-survival curves of SKOV3ip.Fluc-bearing mice treated with Stealth-N1E and Stealth-A09 viruses (n=5). Statistical significance defined by long-rank test. (C) Representative BLI showing the dorsal view of treated animals. Radiance (photons per second per cm per steradian, p/s/cm²/sr) was translated to colors to indicate tumor burden in the mice, according to the legend shown on the right. (D) Quantification of the total body luminescence in photons per second per cm per steradian (p/s/cm²/sr). n=5. Ns, not significant; *, p-value <0.05; **, p<0.005.

FIG. 25. Stealth-A09 virus achieves oncolysis indistinguisible from the parental MeV in a mouse model of multiple myeloma. (A) SCID mice bearing subcutaneous U266.B1 cell tumors were treated intravenously with a suboptimal dose of virus. Tumor growth was measured with a caliper (n=5), and animals were euthanized when the tumors ulcerated or when the tumor size reached 20% of the body weight. (B) Kaplan-Meier survival curves (n=5). Significant differences among the groups were determined by the long-rank test (*, p<0.05). (C) Virus trafficking to subcutaneous tumor cells after systemic administration. eGFP expression was evaluated by immunohistochemistry of two representative samples from each group collected at euthanasia. Scale, 200 nm.

FIG. 26. Increased binding affinity to CD46 enhances CD46-specific virus entry. Fluc-expressing Stealth viruses were used to infect the indicated cells at decreasing MOI. Luciferase expression was measured 48 hours post-infection. n=2 for all except CHO-CD46 and Stealth-A09 (n=3).

FIG. 27. Stealth virus remains oncolytic in the presence of MeV-immune serum. (A) SKOV3ip.Fluc cells were injected into athymic nude mice and allowed to establish for 10 days. Next, mice in the relevant groups received 600 mIU of anti-MeV IgG antibody intraperitoneally three hours before virus treatment via the same route. (B) Kaplan-Meier survival curves (n=5 mice per group). Significant differences among the groups were determined by the long-rank test (ns, not significant; *, p<0.05; **, p<0.002) (C) Representative BLI showing the dorsal view of treated animals. Radiance (photons per second per cm per steradian, p/s/cm²/sr) was translated to colors to indicate tumor burden in the mice, according to the legend shown on the right. (D) Quantification of the total body luminescence in photons per second per cm per steradian (p/s/cm²/sr). n=5. Statistical significance was determined by one-way ANOVA with Dunnetts' multiple comparison test. Ns, no significant.

FIG. 28. Lack of cross-neutralization between measles virus and Stealth. (A) Virus neutralization assay for MeV and Stealth. Human AB pooled serum (left panel) or ferret anti-CDV serum (right panel) was used. Relative infection refers to the amount of infection in the presence of serum compared with that in the absence of serum. Values were calculated from two or three biological replicates performed in technical quadruplicates. (B) Antisera from infected HuCD46Ge-IFNARKO mice was also used to determine the cross-neutralization between the viruses, n=8 (note that some data points overlap). ND₅₀ titers were converted to mIU/mL based on the ND₅₀ obtained for MeV when assessed with the 3rd WHO International serum standard (3 IU/mL).

### DETAILED DESCRIPTION

This document provides CDV F polypeptides. As described herein, a CDV F polypeptide can be designed such that virus particles containing the CDV F polypeptide together with a CDV H polypeptide exhibit enhanced fusogenic activity. For example, a CDV F polypeptide can be designed to contain a signal peptide sequence that is no longer than 75 amino acids in length. Typically, wild-type CDV F polypeptides contain a signal peptide sequence that is about 135 amino acids in length. An example of a 135 amino acid signal peptide sequence of a wild-type CDV F polypeptide is set forth in SEQ ID NO:6 (MHKEIPEKSRTRTHTQQDLPQQKSTEYTEIKTSRARHGITPAQRSTH YGPRTLDRLVCYIMNRAMSCKQASYRSDNIPAHGDHEGVVHHTPESVSQGARSQ LKRRTSNAINSGFQYIWLVLWCIGIASLFLCSKA). As described herein, truncating the signal peptide sequence of CDV F polypeptides such that it is no longer than 75 amino acids in length can result in CDV F polypeptides that, when part of viruses together with CDV H polypeptides, allow for increased fusogenic activity of the viruses as compared to the level of fusogenic activity exhibited by comparable control viruses containing a CDV F polypeptide having a full-length wild-type signal peptide sequence (e.g., SEQ ID NO:6).

A CDV F polypeptide provided herein can contain a signal peptide sequence that is from 7 amino acids to 75 amino acids in length. For example, a CDV F polypeptide provided herein can contain a signal peptide sequence that is from 7 to 75 (e.g., from 7 to 70, from 7 to 65, from 7 to 60, from 7 to 55, from 7 to 50, from 7 to 45, from 7 to 40, from 7 to 35, from 7 to 30, from 7 to 25, from 10 to 75, from 15 to 75, from 20 to 75, from 25 to 75, from 35 to 75, from 45 to 75, from 50 to 75, from 55 to 75, from 65 to 75, from 20 to 60, from 25 to 50, from 30 to 60, or from 30 to 40) amino acids in length. A CDV F polypeptide provided herein can be produced by truncating a wild-type signal peptide sequence from its N-terminus, from its C-terminus, or from both its N-terminus and C-terminus or by deleting amino acids from in between the N-terminal and C-terminal regions of a wild-type signal peptide sequence. In some cases, a measles virus signal peptide sequence can be used for a signal peptide of a CDV F polypeptide described herein. Examples of signal peptide sequences of CDV F polypeptides provided herein include, without limitation, those set forth in Table 1.

TABLE 1
Examples of signal peptide sequences.

| Ex- | | SEQ |
|---|---|---|
| ample | | ID |
| # | Sequence | NO: |
| 1 | MNRAMSCKQASYRSDNIPAHGDHEGVVHHTPESVSQG | 8 |
| | ARSQLKRRTSNAINSGFQYIWLVLWCIGIASLFLCSKA | |
| 2 | MTSNAINSGFQYIWLVLWCIGIASLFLCSKA | 9 |
| 3 | MSIMGLKVNVSAIFMAVLLTLQTPTG | 10 |
| 4 | MAINSGSQCTWLVLWCLGIASLFLCSKA | 11 |
| 5 | MAINSGSQCTWLVLWCLGMASLFLCSKA | 12 |
| 6 | MAINSGSQCTWLVLWCFGTASLFLCSKA | 13 |
| 7 | MATSPGPQCTWLVLWCIGIASLFLCSEA | 14 |
| 8 | MAVKSGSQCTWLVLWCIGVASLFLCSKA | 15 |
| 9 | MASNPGSQCTWLVLWCIGIASLFLCSKA | 16 |
| 10 | MATNAGSQYTWLVLWCIGIASLFLCSKA | 17 |
| 11 | MATSSGSQCTWLVLWCIGVASLFLCSKA | 18 |
| 12 | MAINSGSQCTWLVLWCIGVASLFLCSKA | 19 |
| 13 | MAINSGFQYIWLVLWCIGIASLFLCSKA | 20 |

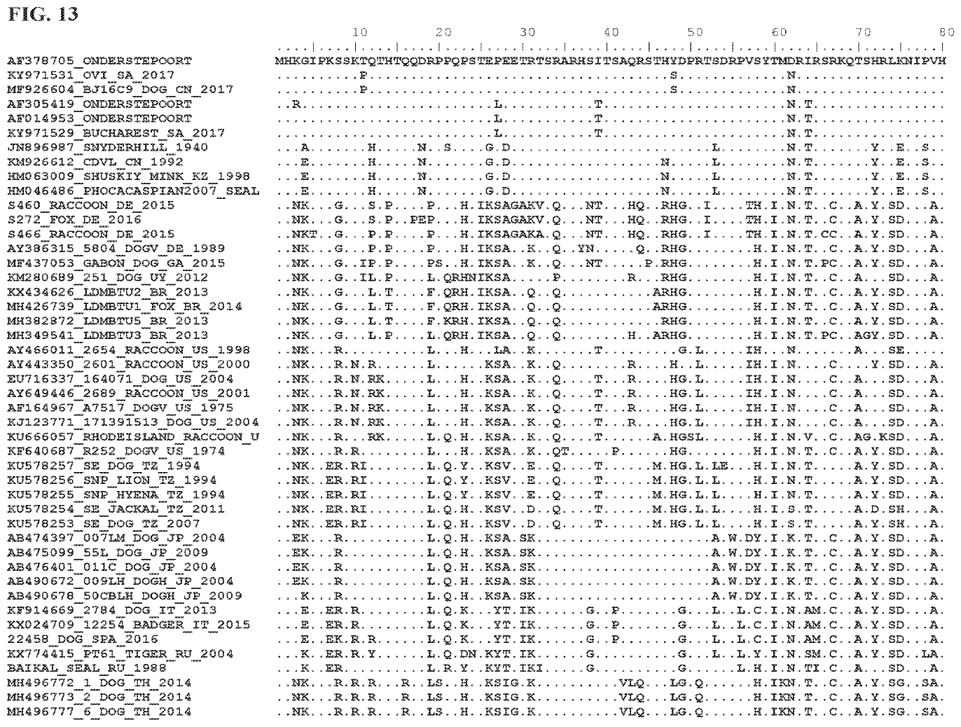

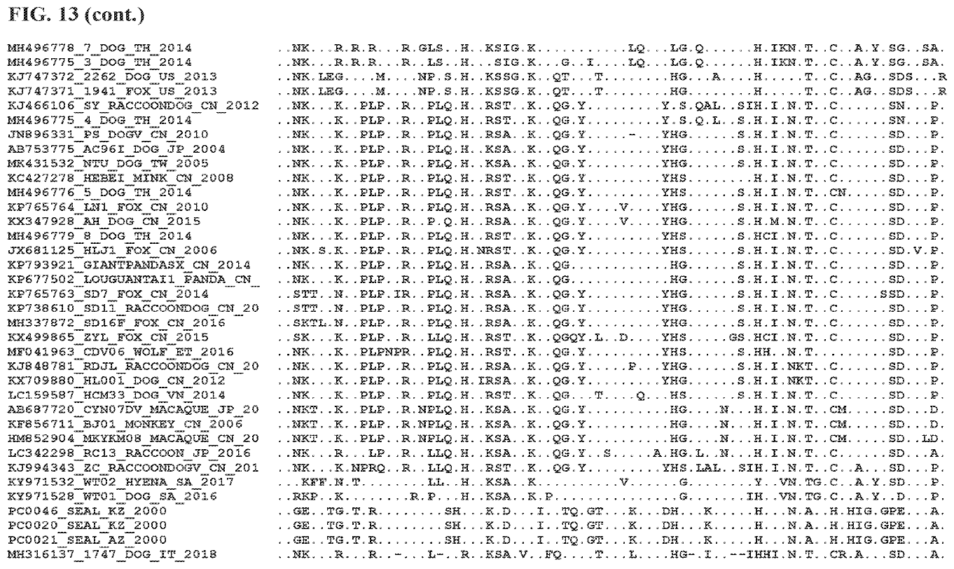

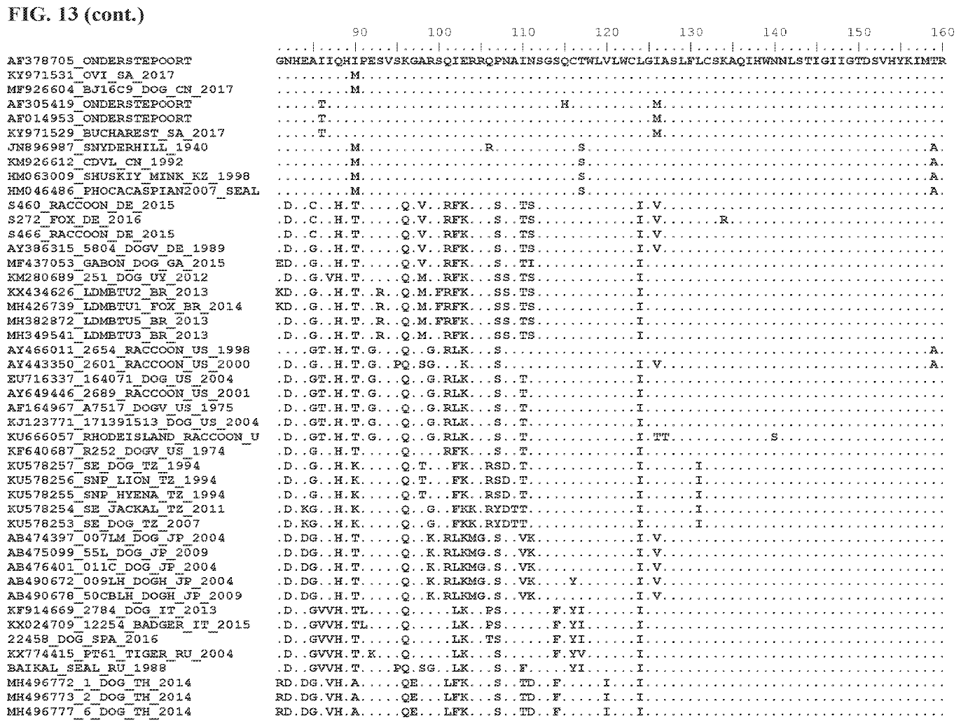

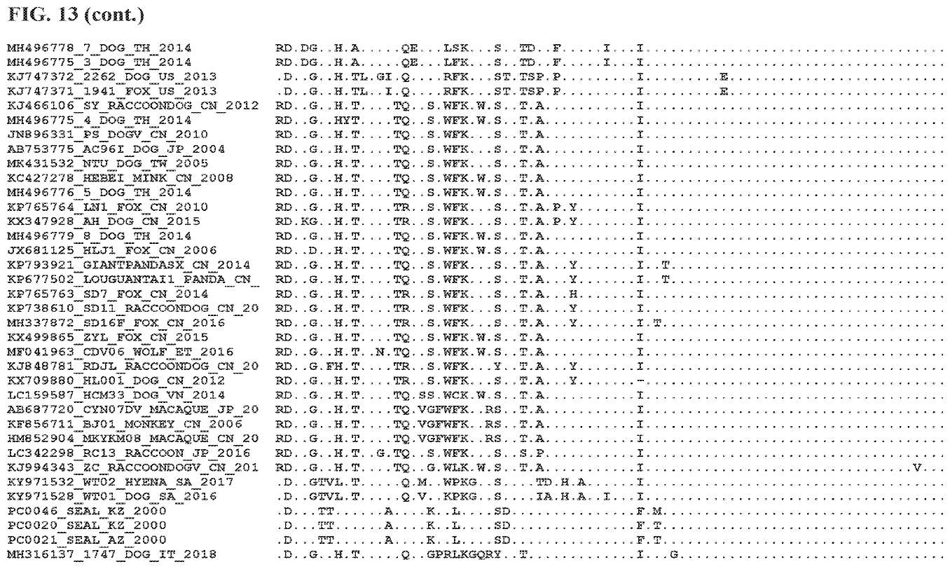

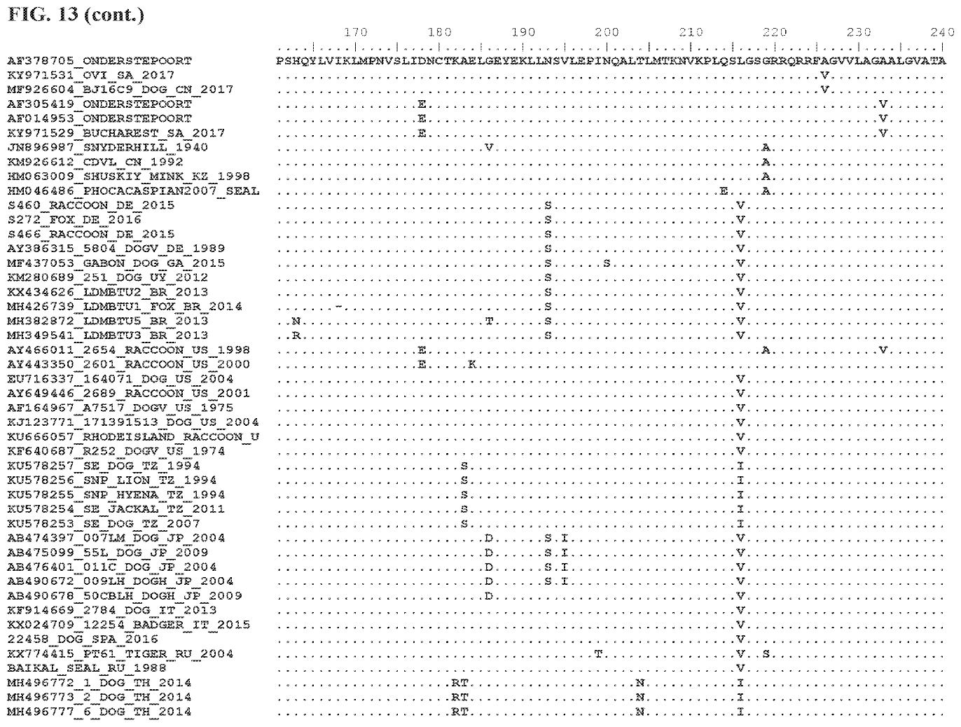

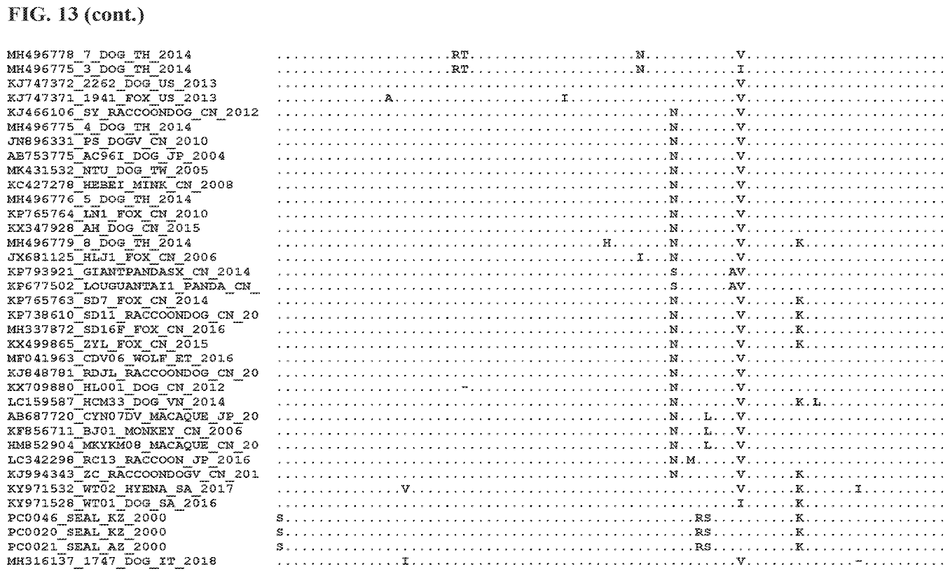

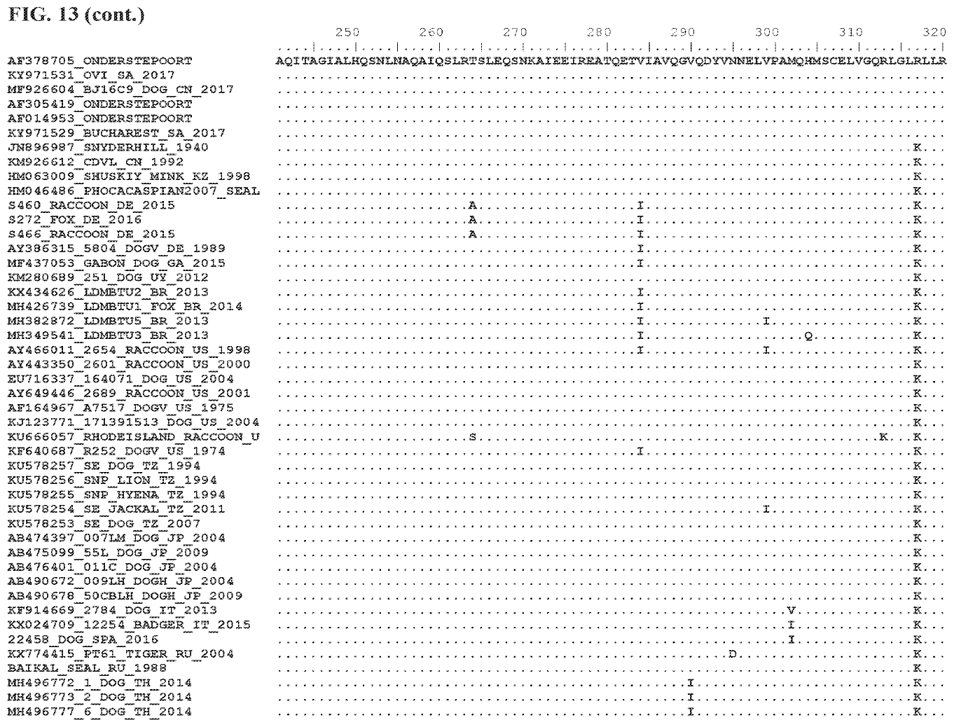

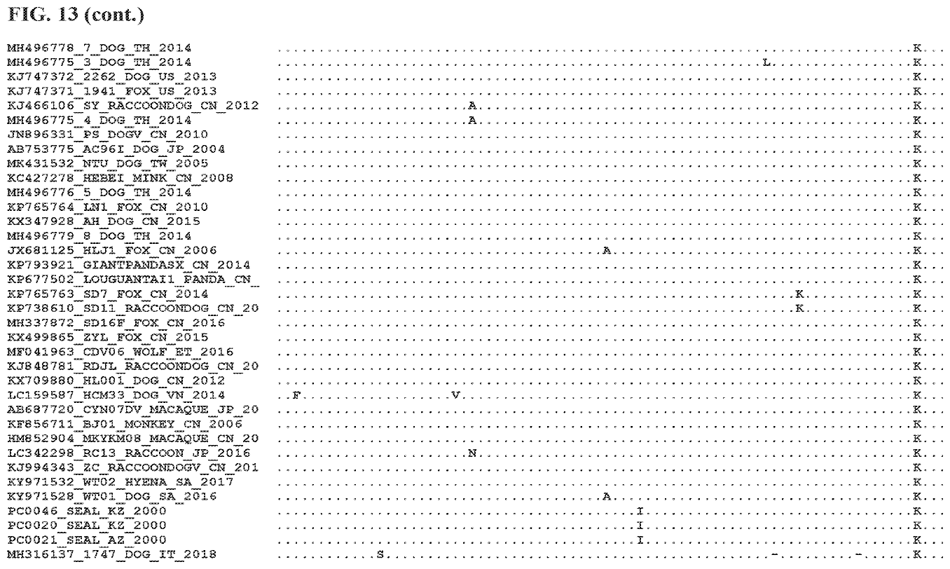

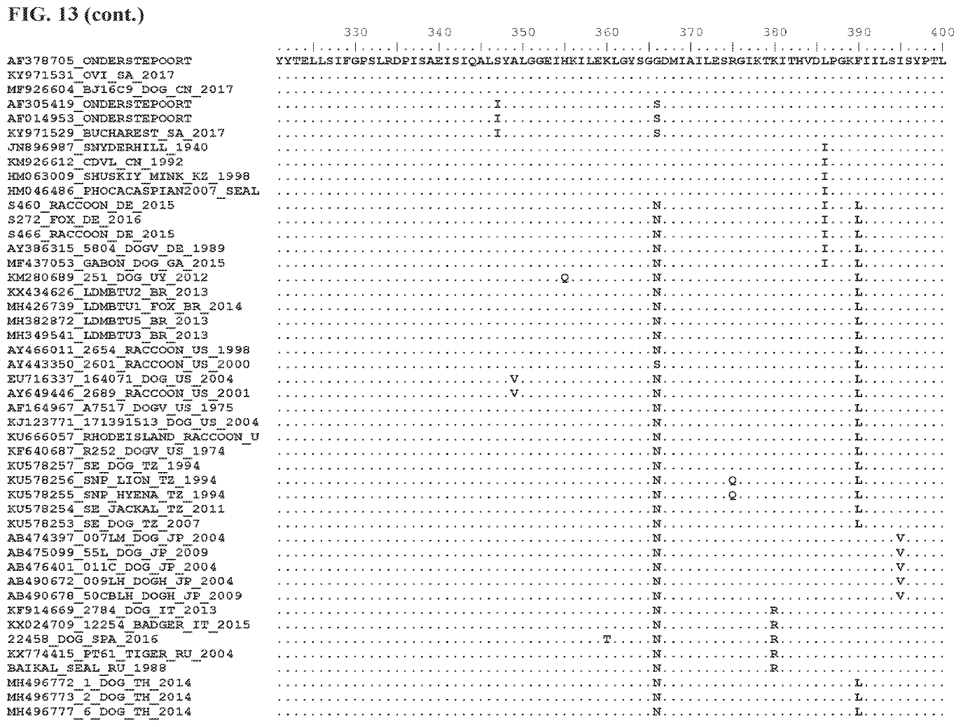

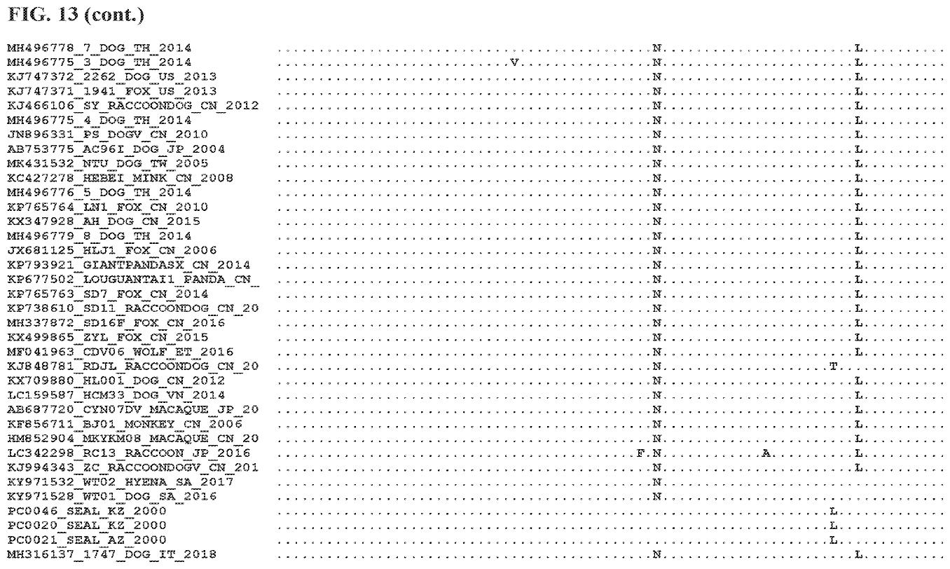

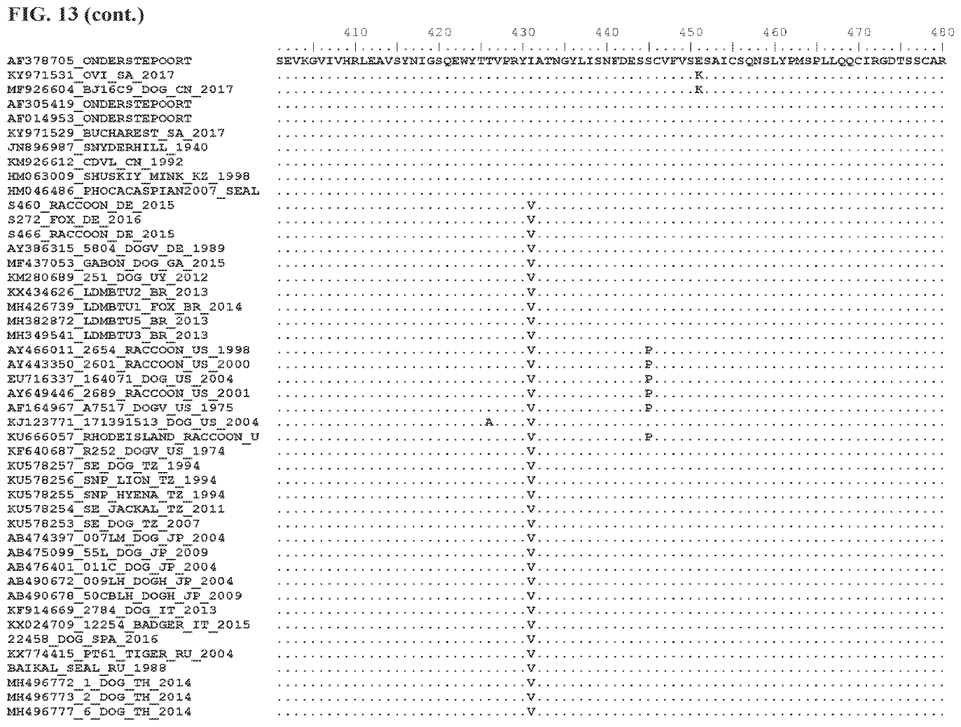

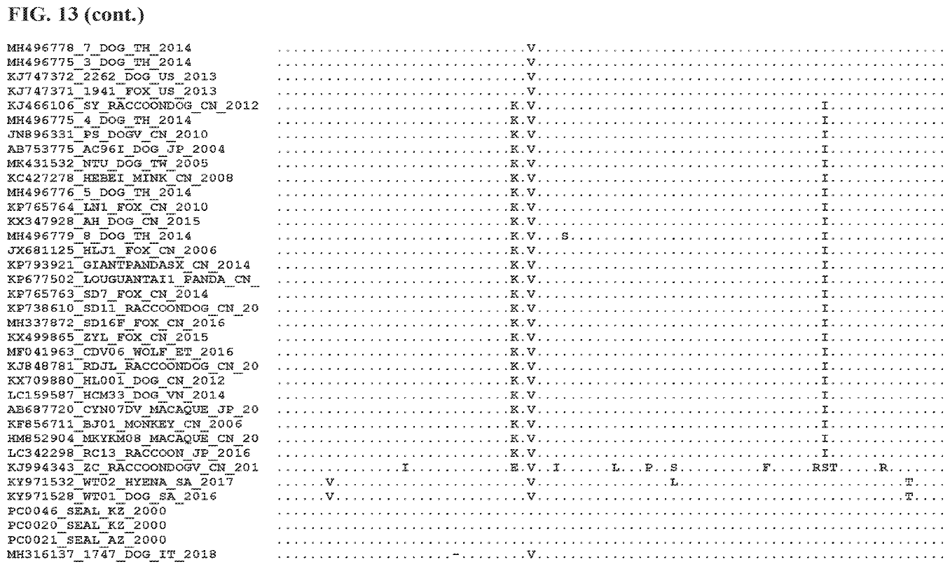

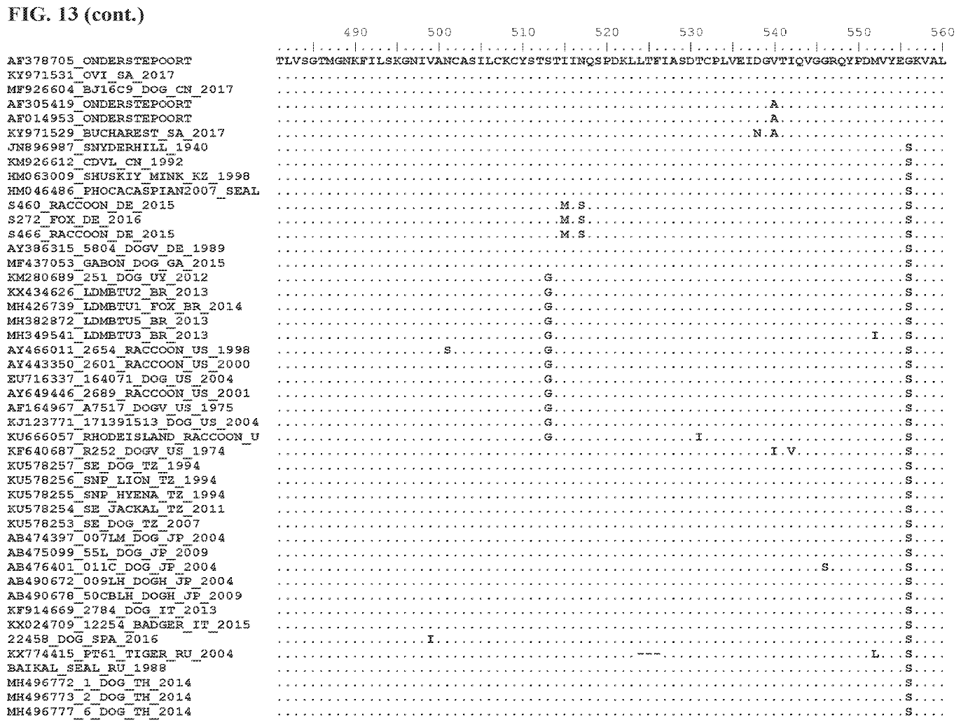

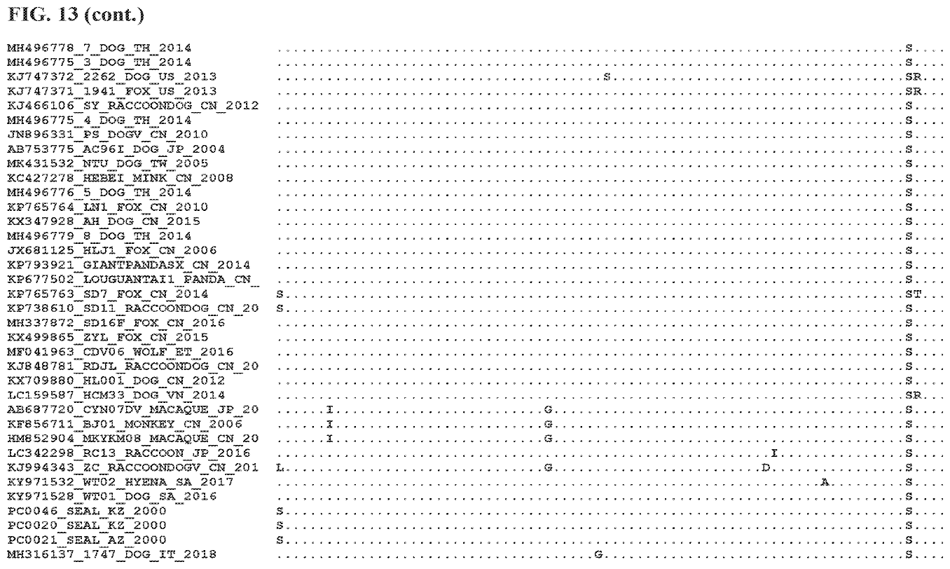

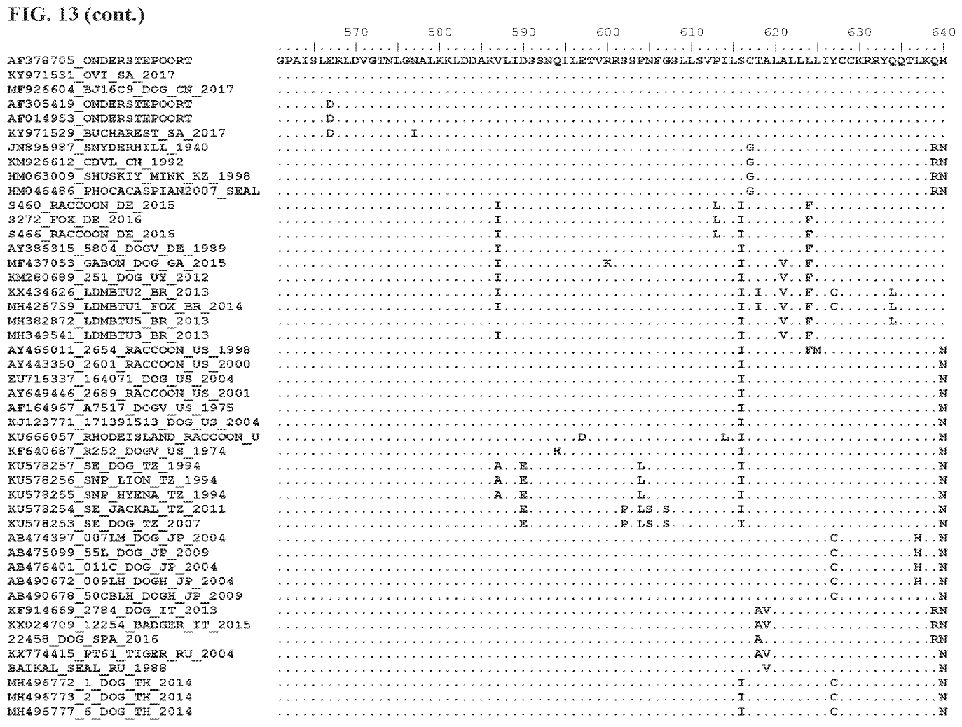

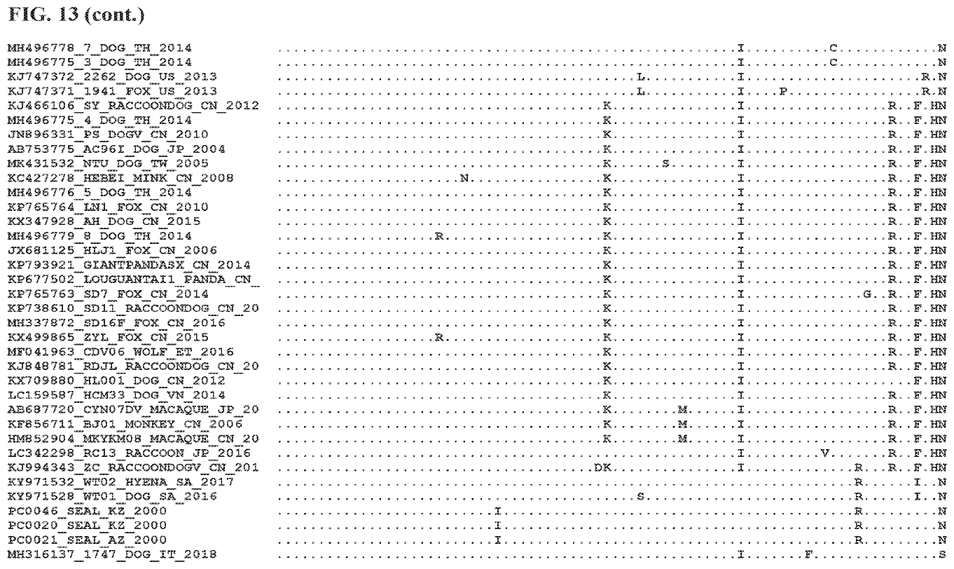

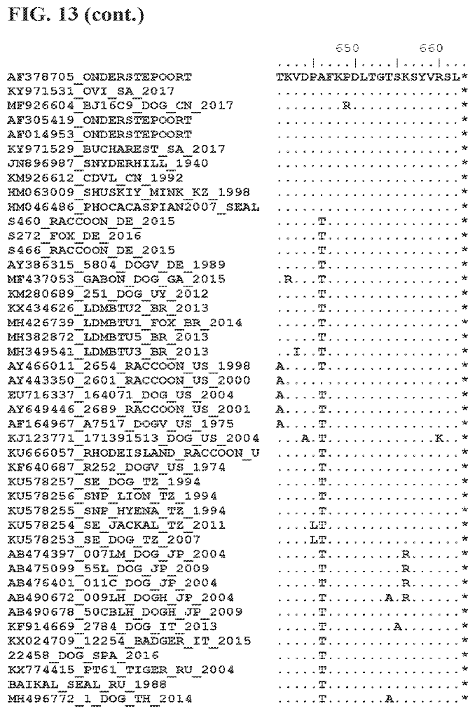

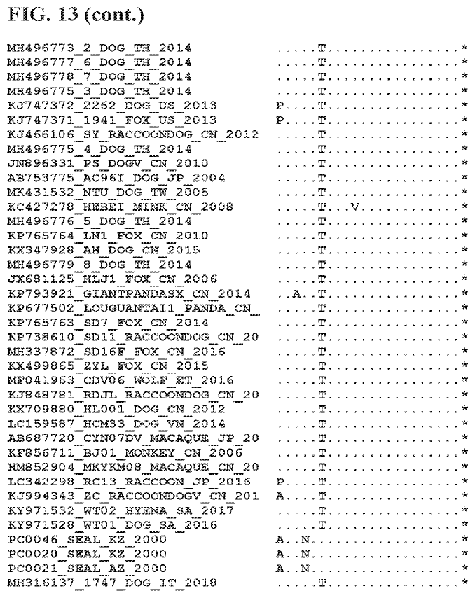

In some cases, a CDV F polypeptide provided herein can be designed to lack the entire signal peptide sequence. For example, a CDV F polypeptide provided herein can have one of the amino acid sequences set forth in FIG. 13 starting with the amino acid at position 140.

A CDV F polypeptide provided herein can have any appropriate amino acid sequence provided that the CDV F polypeptide does not contain a signal peptide sequence longer than 75 amino acid residues in length. Examples of amino acid sequences of CDV F polypeptides that can be used as described herein include, without limitation, those amino acid sequences set forth in FIG. 13.

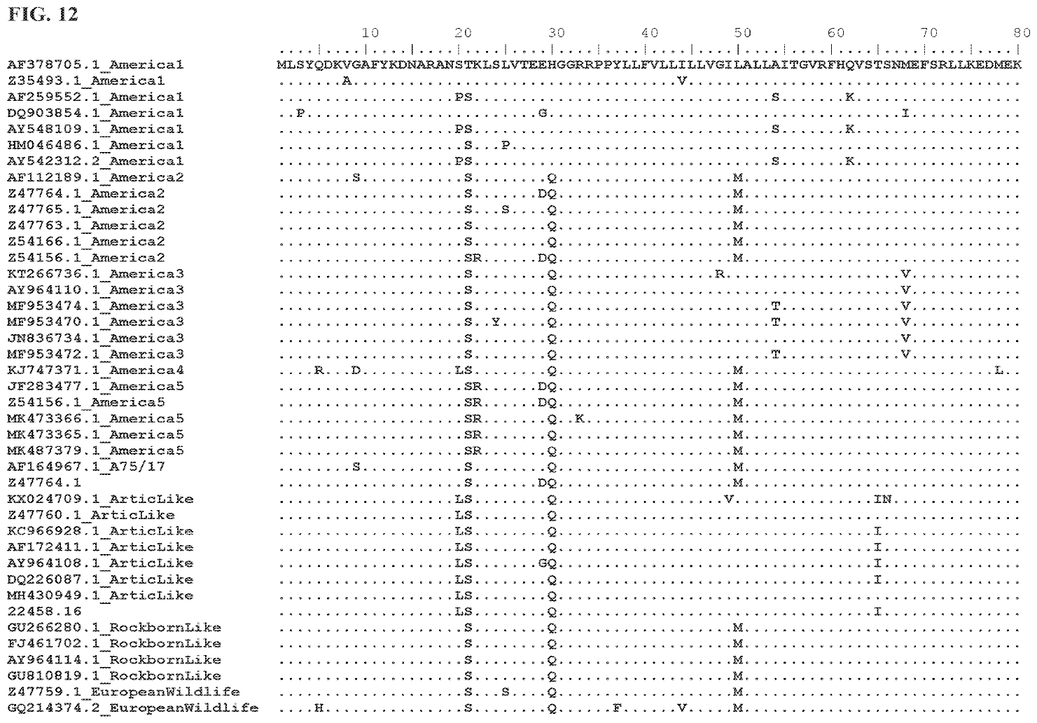

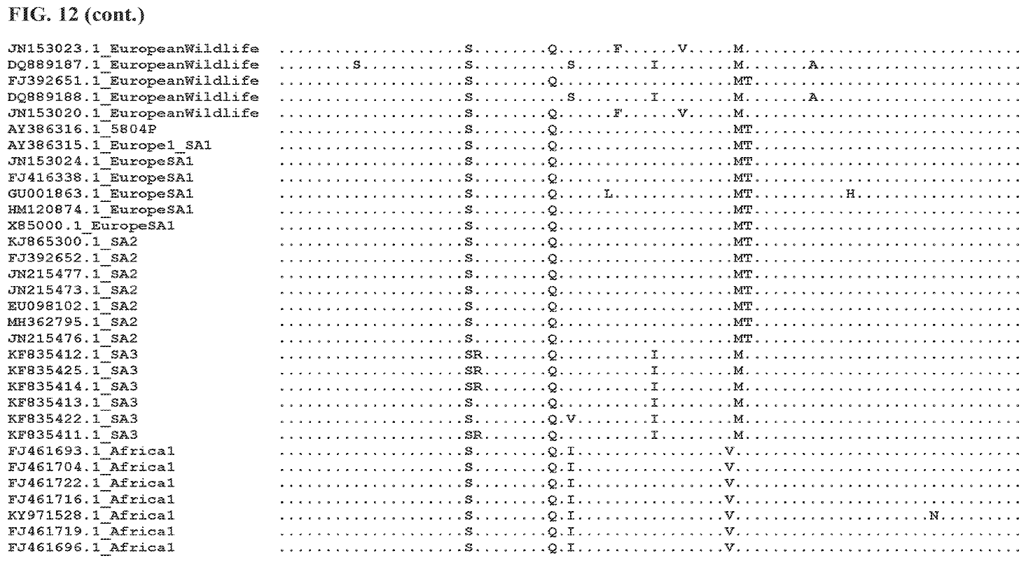

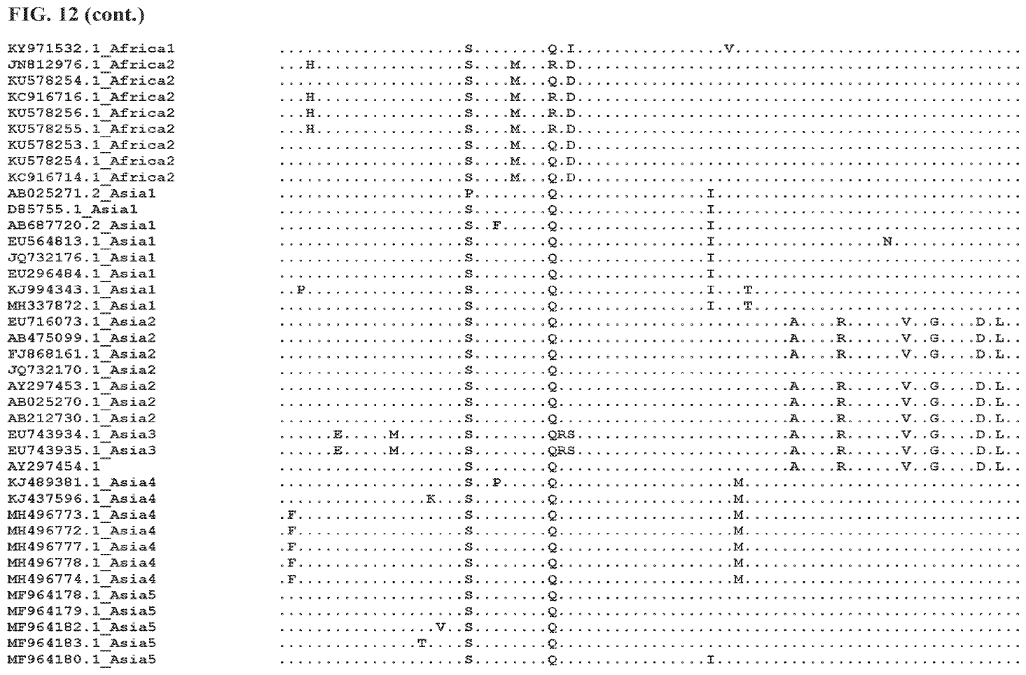

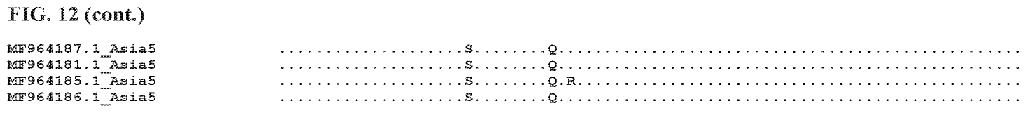

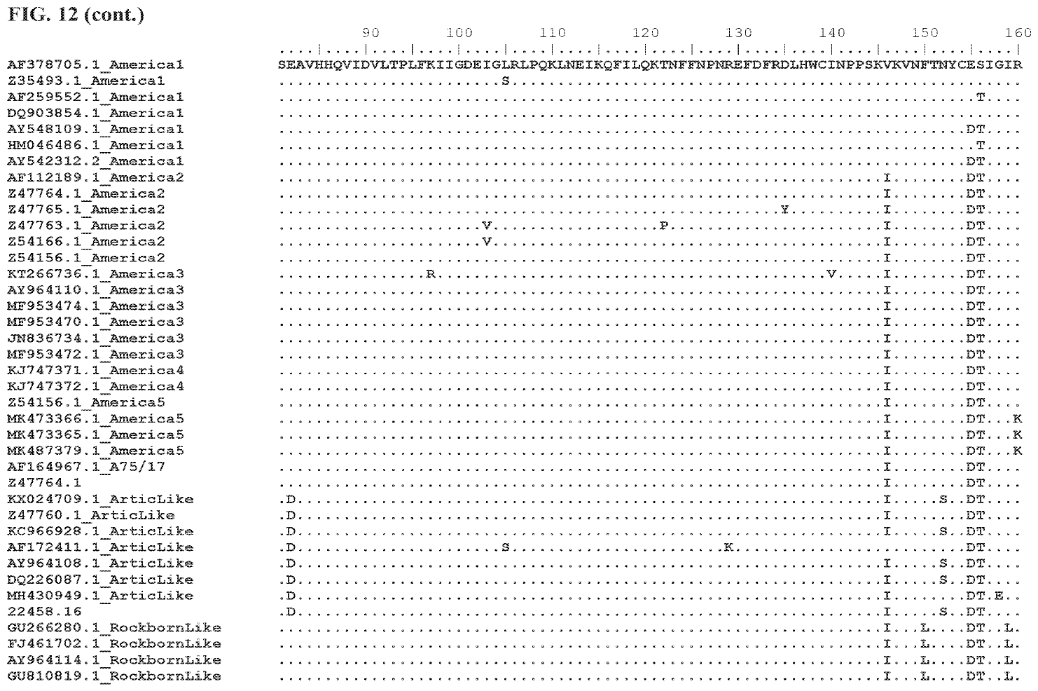

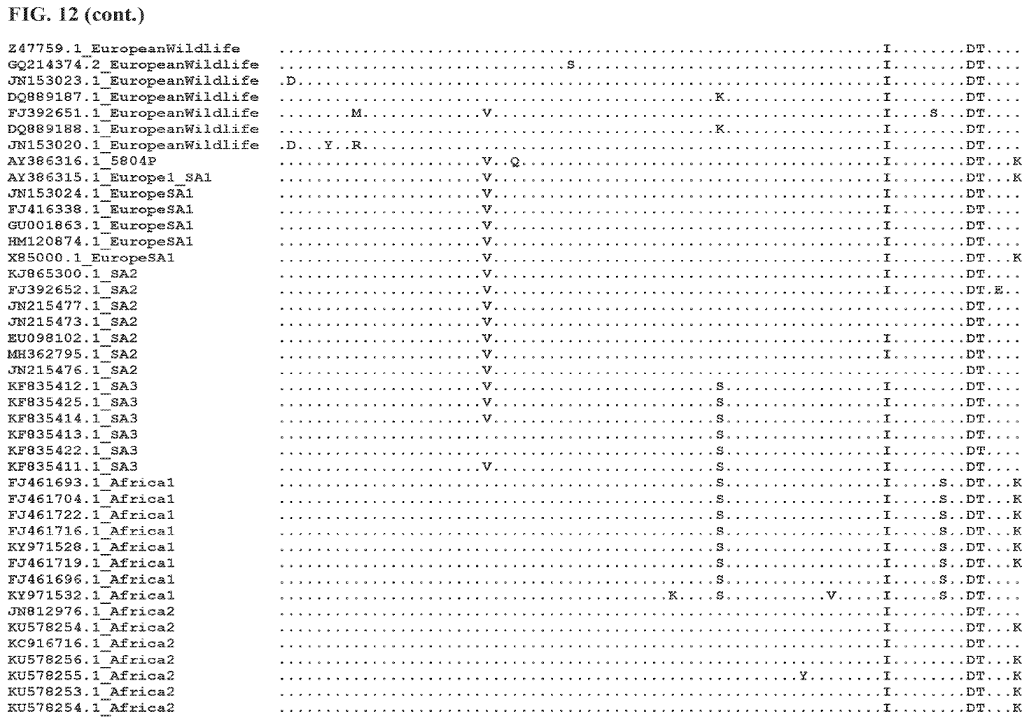

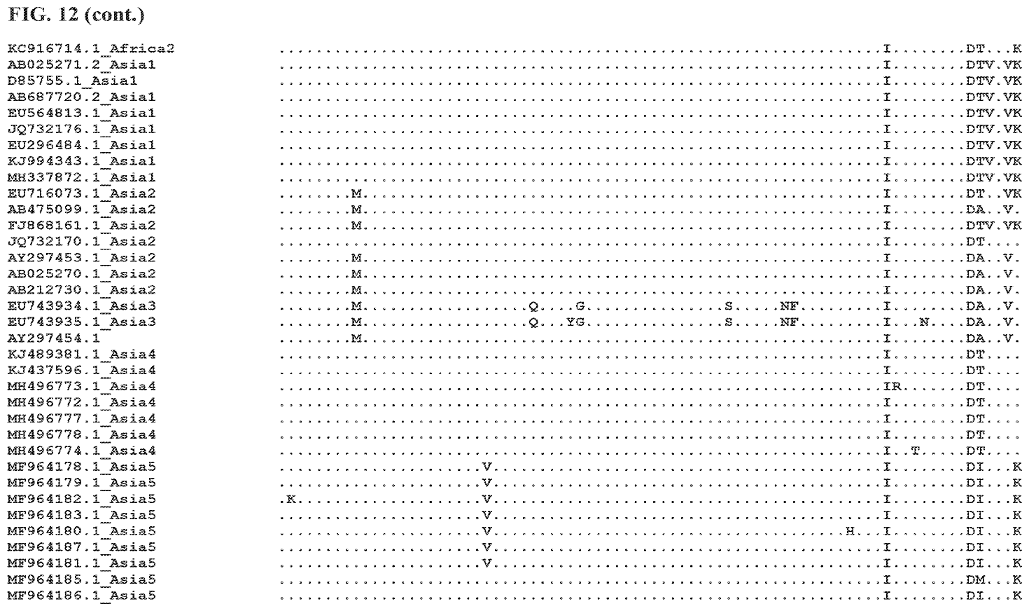

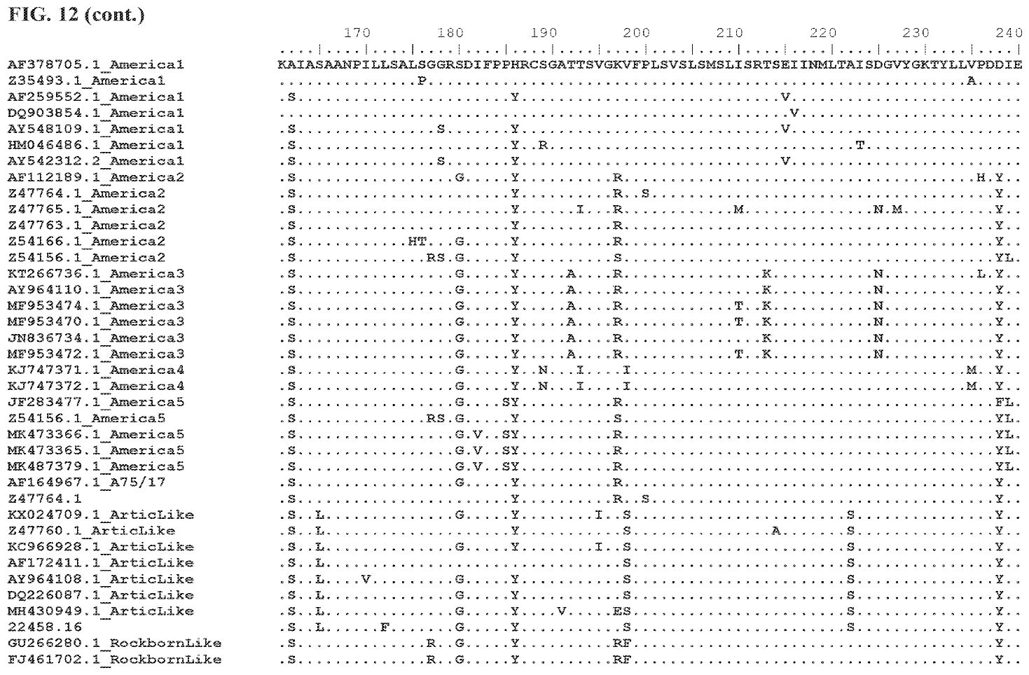

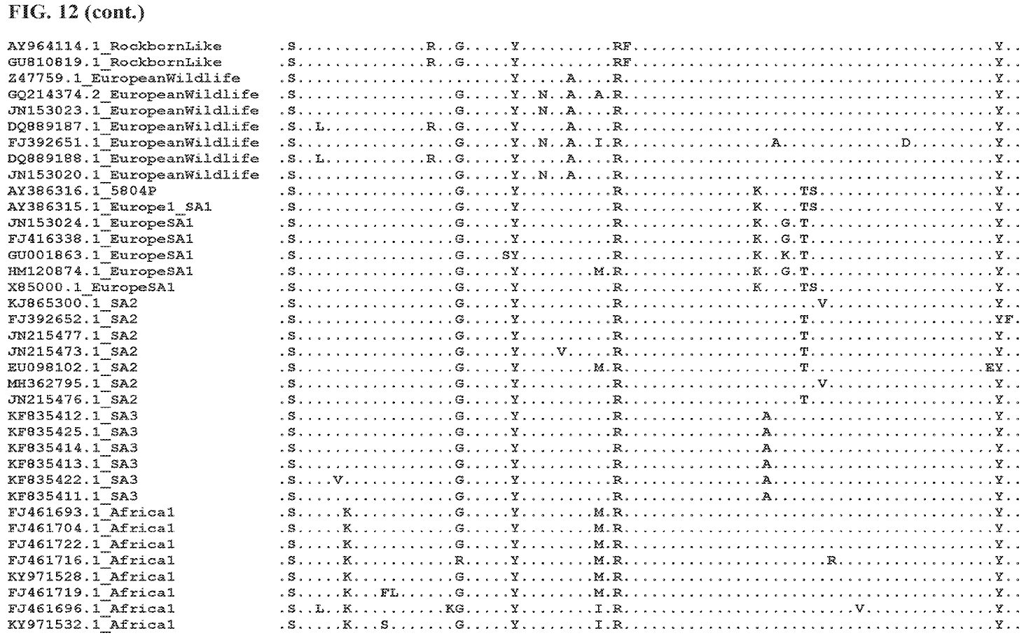

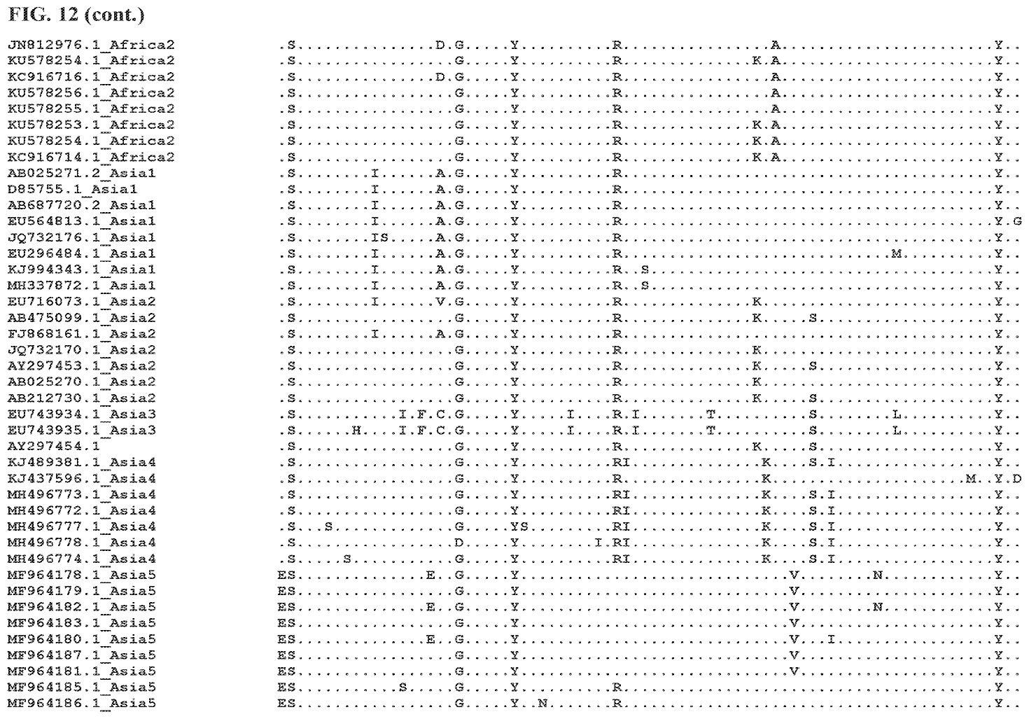

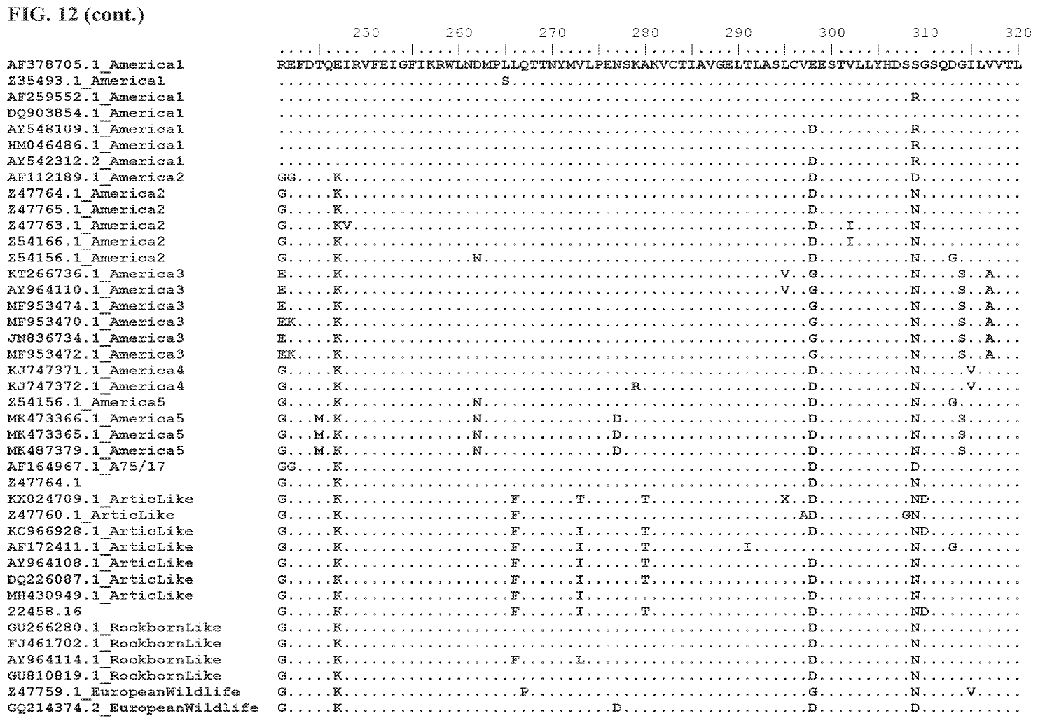

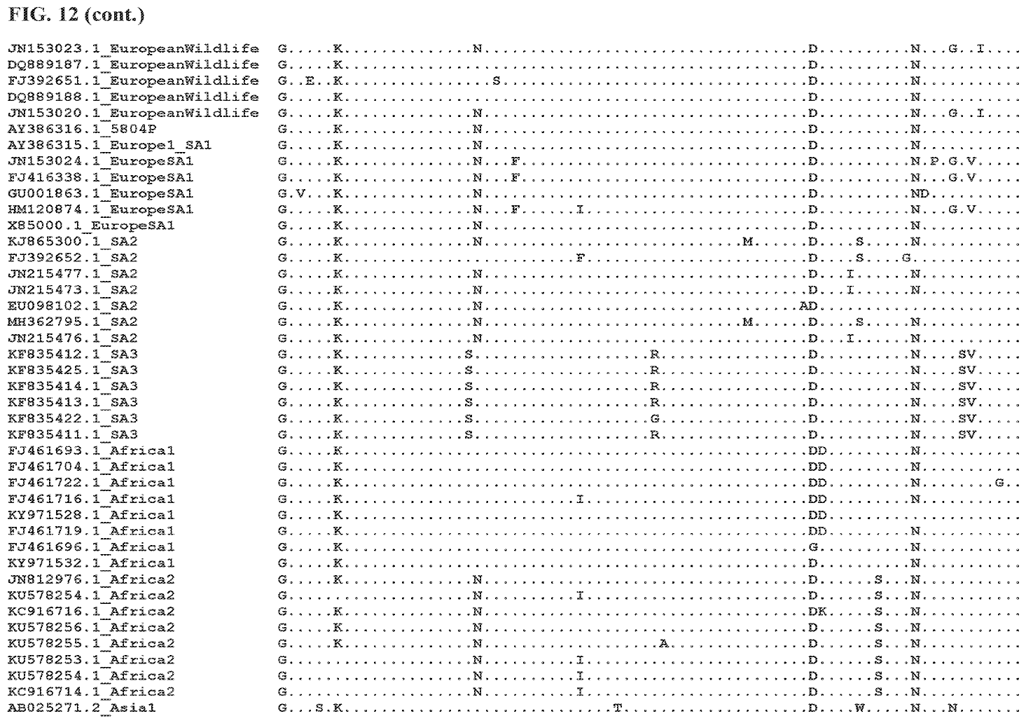

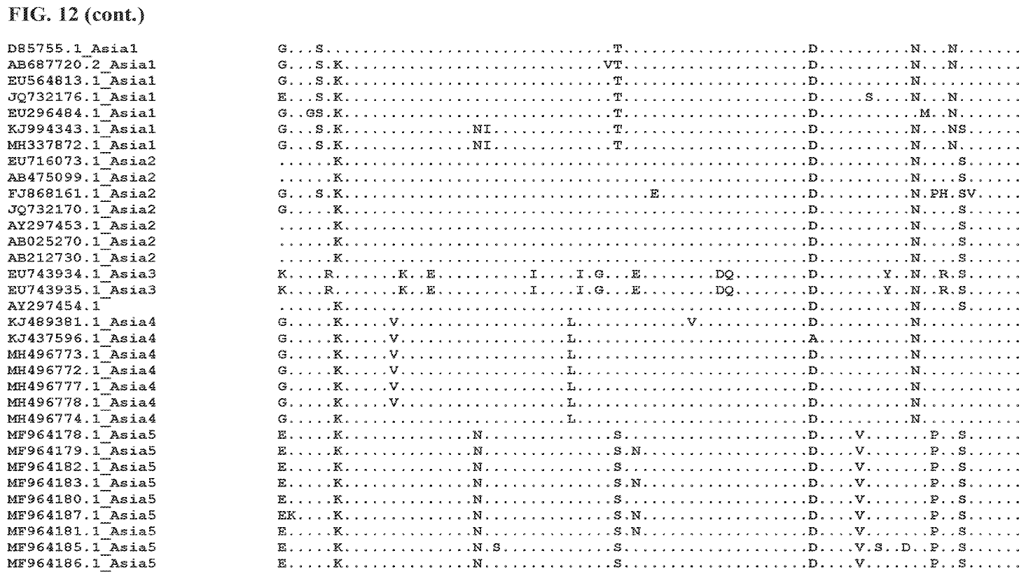

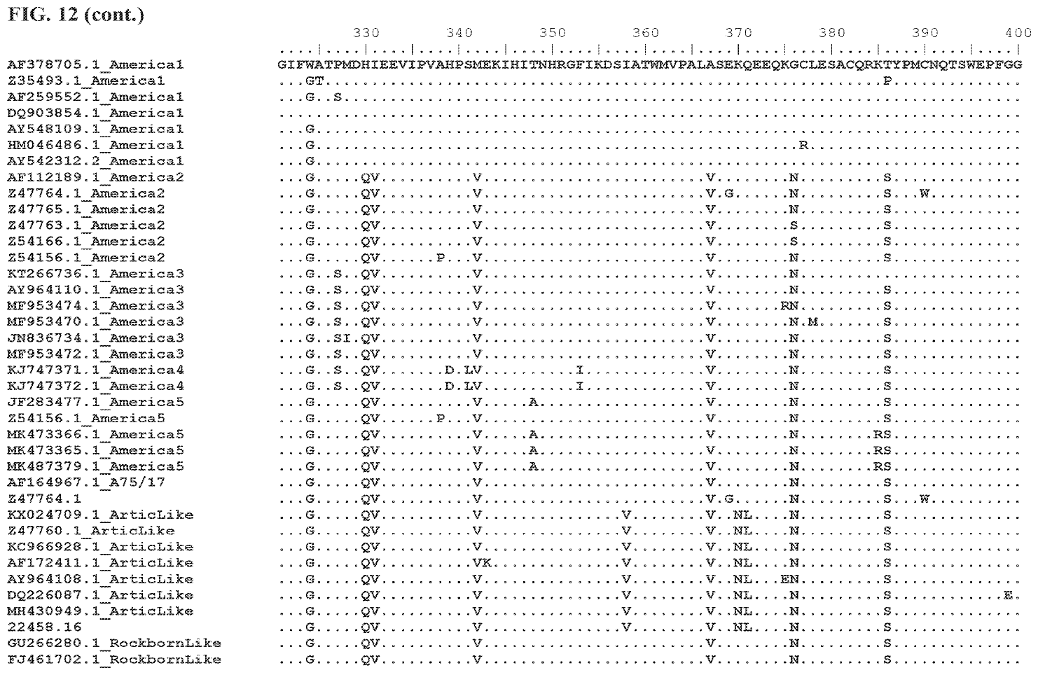

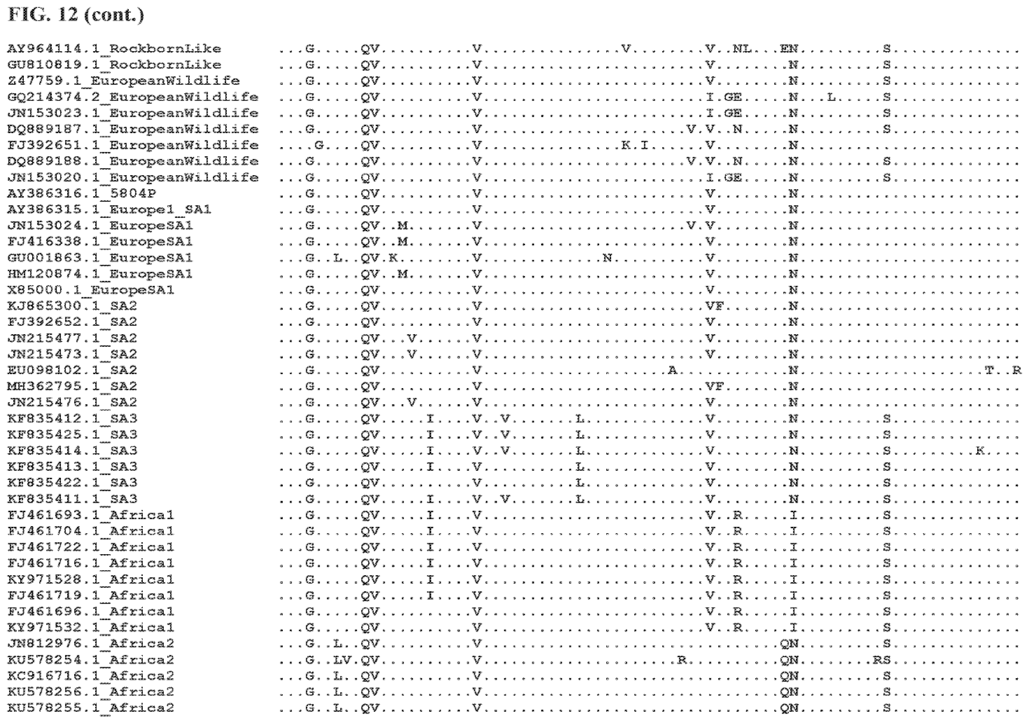

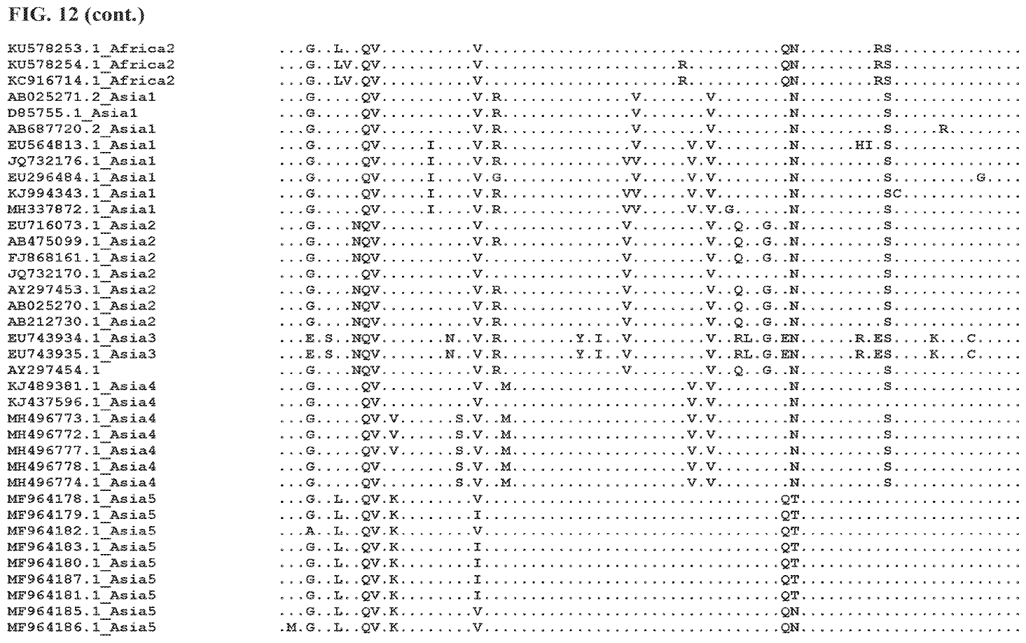

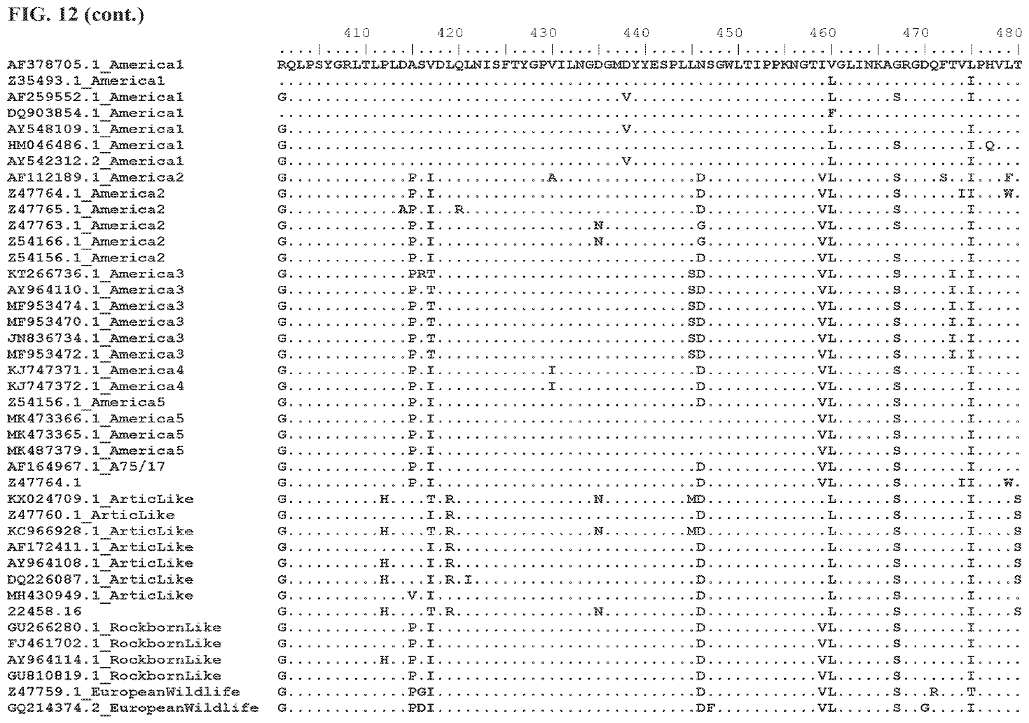

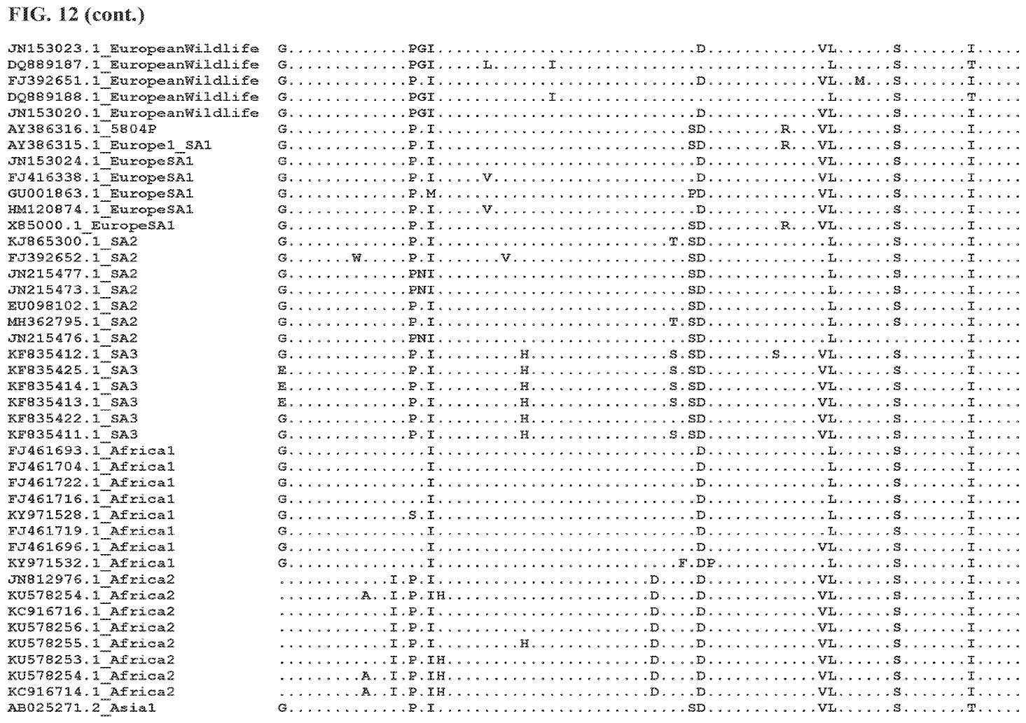

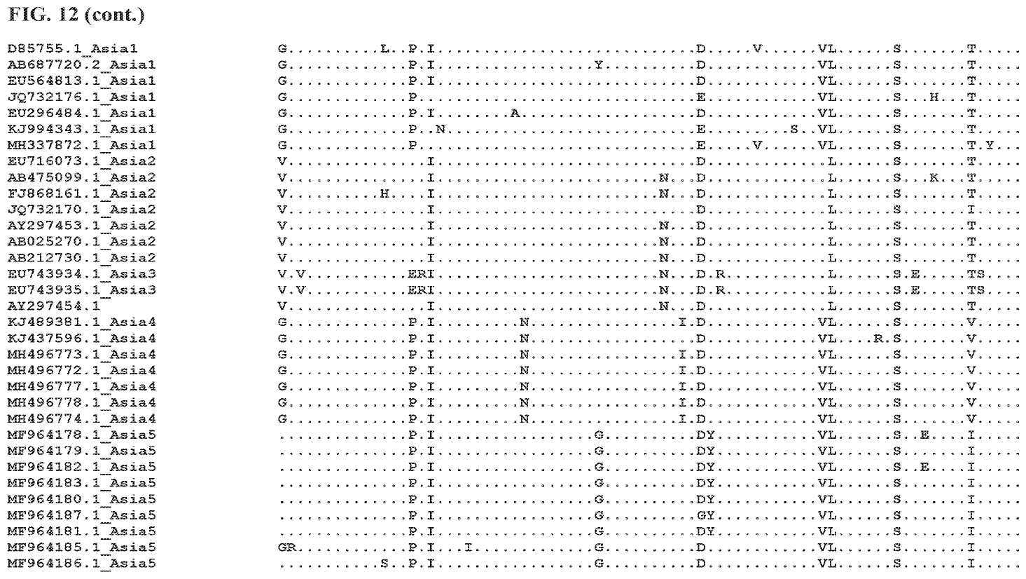

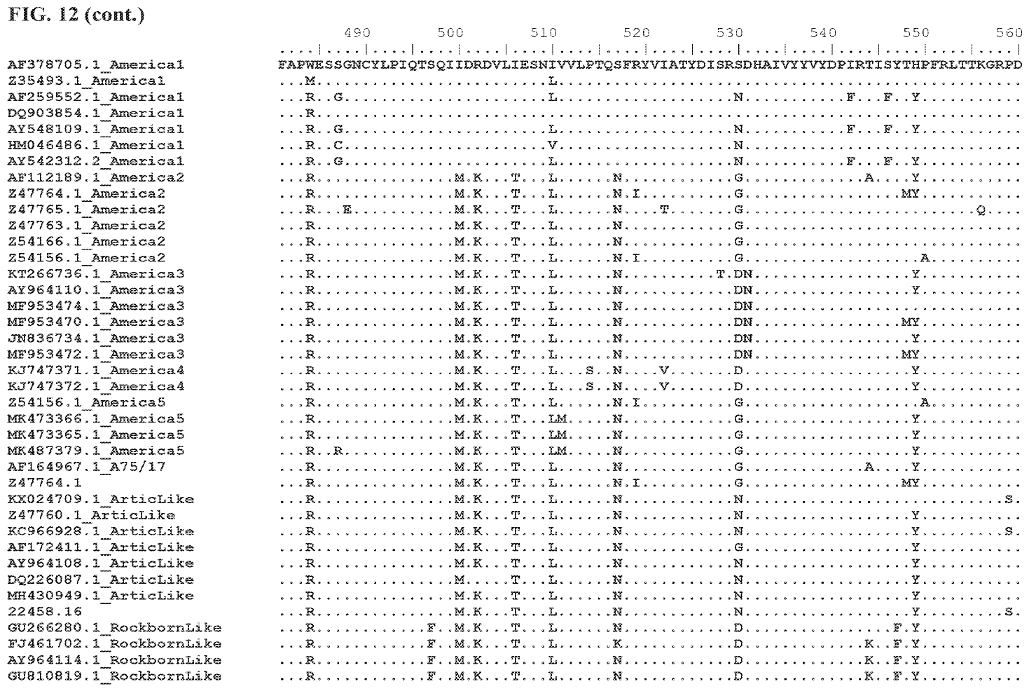

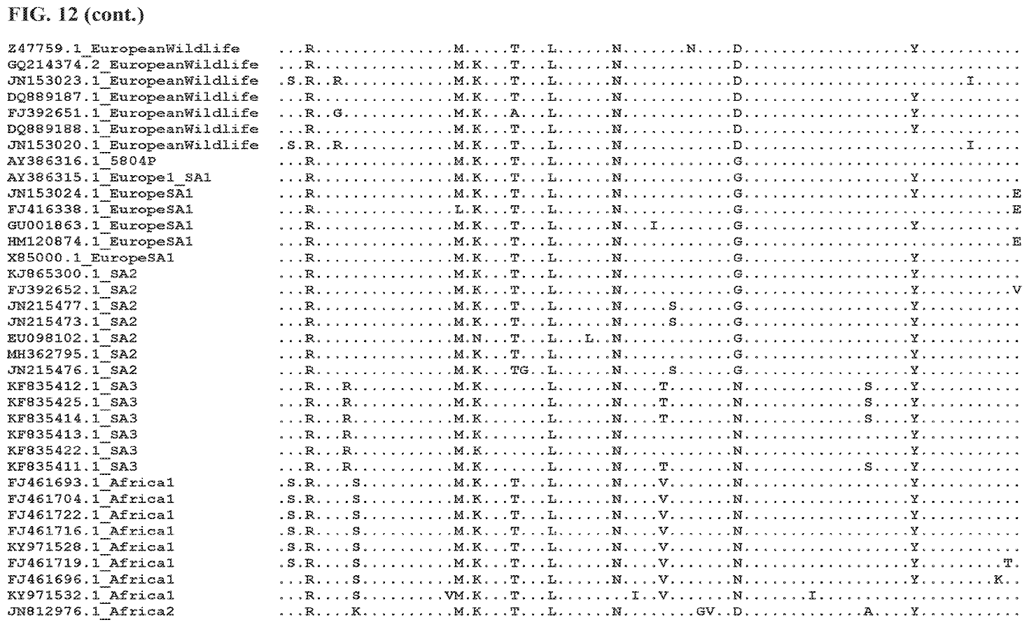

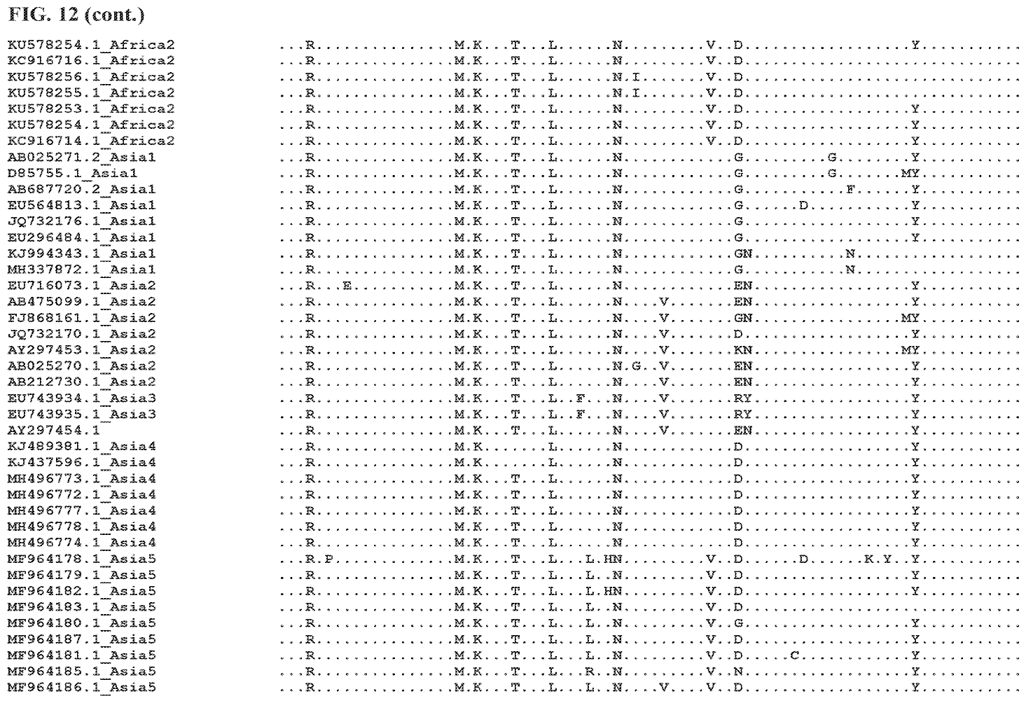

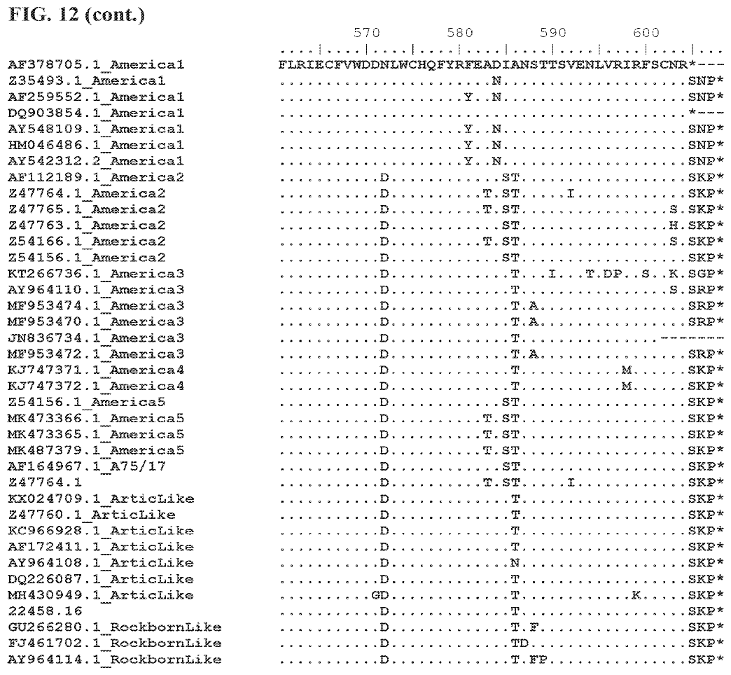

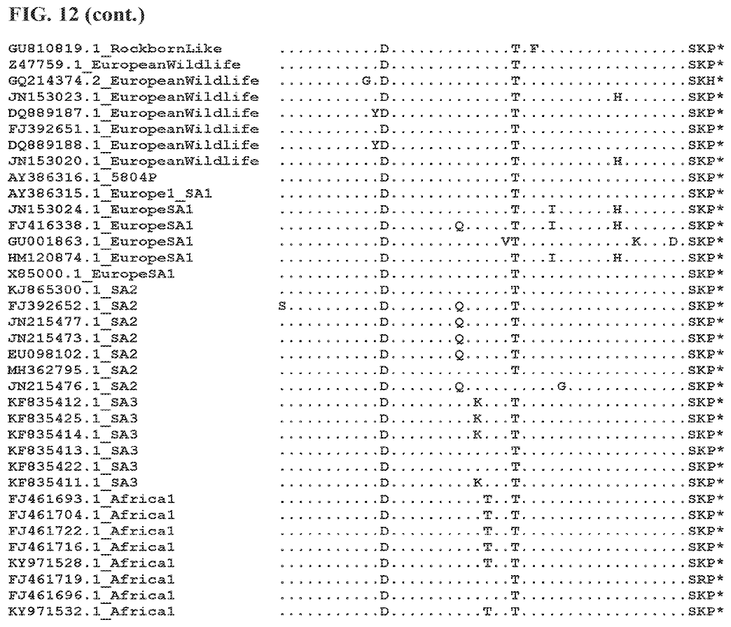

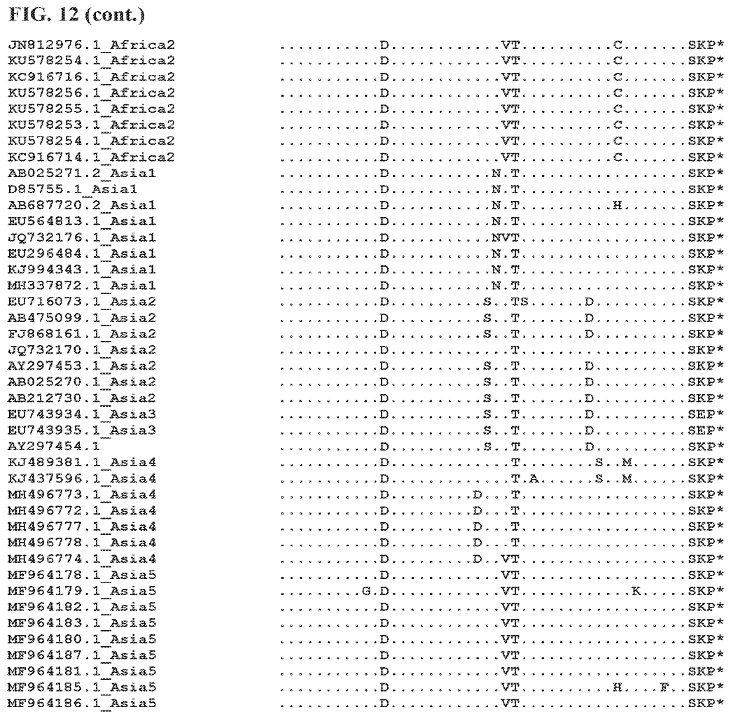

This document also provides CDV H polypeptides. As described herein, a CDV H polypeptide can be designed such that viruses containing the CDV H polypeptide together with a CDV F polypeptide exhibit reduced or eliminated tropism for SLAMF1 polypeptides and/or Nectin-4 polypeptides as compared to viruses containing wild-type CDV H polypeptides. For example, a CDV H polypeptide can be designed to contain a mutation at one or more (e.g., one, two, three, four, five, six, seven, eight, nine, ten, 11, 12, 13, 14, or 15) of amino acid positions 454, 460, 479, 494, 510, 520, 525, 526, 527, 528, 529, 537, 539, 547, and 548. Typically, viruses containing wild-type CDV H polypeptides (together with CDV F polypeptides) exhibit tropism for SLAMF1 polypeptides and Nectin-4 polypeptides such that the viruses infect SLAMF1-positive cells and Nectin-4-positive cells. As described herein, mutating one or more of amino acid positions P/S454, V/L/F460, L/F/W479, I494, I/L/V510, Y520, Y/N525, D/G526, I/V527, S/T528, R529, Y/D537, Y539, Y/F547, and Y/M548 of a CDV H polypeptide to a different amino acid (e.g., alanine) can reduce or eliminate the ability of viruses containing that CDV H polypeptide (together with a CDV F polypeptide) to infect SLAMF1-positive cells and/or Nectin-4-positive cells. Examples of CDV H polypeptides provided herein having reduced or eliminated tropism for SLAMF1 polypeptides and/or Nectin-4 polypeptides include, without limitation, those CDV H polypeptides set forth in FIG. 12 provided that the CDV H polypeptide contains a mutation of one or more (e.g., one, two, three, four, five, six, seven, eight, nine, ten, 11, 12, 13, 14, or 15) of WP/454, V/L/F460, L/F/W479, I494, I/L/N510, Y520, Y/N525, D/G526, I/V527, S/T528, R529, Y/D537, Y539,Y/F547, and Y/M548. Examples of mutations that can be used to make a CDV H polypeptide having reduced or eliminated tropism for SLAMF1 polypeptides and/or NectinS4 polypeptides include, without limitation, those set forth in Table 2. Examples of combinations of the mutations set forth in Table 2 that can be used to make a CDV H polypeptide having reduced or eliminated tropism for SLAMF1 polypeptides and/or Nectin-4 polypeptides include, without limitation, those set forth in Table 3.

TABLE 2
Examples of mutations that can be introduced into a CDV H
polypeptide (e.g., a CDV H polypeptide set forth in Figure 12).

| | | | Specific |
|---|---|---|---|
| Example # | Position | Amino acid change | examples |
| 1 | P454 | Any amino acid other than | A, I, L, M, F, W, |
| | | P and S | Y, V, R, H, K, D, |
| | | | E, S, T, N, Q, C, |
| | | | G or P |
| 2 | V/L/F460 | Any amino acid other than | A, I, M, F, W, Y, |
| | | V, L, and F | S, T, N, Q, C, G, |
| | | | P, R, H, K, D, or |
| | | | E |
| 3 | L/F/W479 | Any amino acid other than | A, I, M, Y, V, S, |
| | | L, F, and W | T, N, Q, C, G, P, |
| | | | R, H, K, D, E |
| 4 | I494 | Any amino acid other than | A, L M, F, W, Y, |
| | | I | V, S, T, N, Q, C, |
| | | | G, P, R, H, K, D, |
| | | | or E |
| 5 | I/L/V510 | Any amino acid other than | A, M, F, W, Y, S, |
| | | I, L, and V | T, N, Q, C, G, P, |
| | | | R, H, K, D, or E |
| 6 | Y520 | Any amino acid other than | A, I, L M, F, W, |
| | | Y | V, S, T, N, Q, C, |
| | | | G, P, R, H, K, D, |
| | | | or E |
| 7 | Y/N525 | Any amino acid other than | A, I, L M, F, W, |
| | | Y and N | V, S, T, Q, C, G, |
| | | | P, R, H, K, D, or |
| | | | E |
| 8 | D/G526 | Any amino acid other than | A, I, L M, F, W, |
| | | D and G | Y, V, S, T, N, Q, |
| | | | C, P, R, H, K, or |
| | | | E |
| 9 | I/V527 | Any amino acid other than | A, L M, F, W, Y, |
| | | I and V | S, T, N, Q, C, G |
| | | | P, R, H, K, D, or |
| | | | E |
| 10 | S/T528 | Any amino acid other than | A, I, L M, F, W, |
| | | S and T | Y, V, N, Q, C, G, |
| | | | P, R, H, K, D, or |
| | | | E |
| 11 | R529 | Any amino acid other than | A, I, L M, F, W, |
| | | R | Y, V, S, T, N, Q, |
| | | | C, G, P, H, K, D, |
| | | | or E |
| 12 | Y/D537 | Any amino acid other than | A, S, T, N, Q, G, |
| | | Y and D | C, P, D, R, H, K, |
| | | | or E |
| 13 | Y539 | Any amino acid other than | A, S, T, N, Q, G, |
| | | Y | C, P, D, E, R, H, |
| | | | K, or E |
| 14 | Y/F547 | Any amino acid other than | A, S, T, N, Q, G, |
| | | Y and F | C, P, D, E, R, H, |
| | | | K, or E |
| 15 | Y/M548 | Any amino acid other than | A, S, T, N, Q, G, |
| | | Y and M | C, P, D, E, R, H, |
| | | | K, or E |

TABLE 3
Examples of combinations of mutations from Table 2 that can be
included within a CDV H polypeptide.

| Combination # | Combination of mutations from Table 2 |
|---|---|
| 1 | All of 1-15 |
| 2 | 8, 9, 10, 11, 14, and 15 |
| 3 | 7, 8, and 11 |
| 4 | 7 and 8 |
| 5 | 7 and 11 |
| 6 | 8 and 11 |

This document also provides recombinant viruses (e.g., VSVs) containing a CDV H polypeptide provided herein and/or a CDV F polypeptide provided herein as well as methods for making recombinant viruses (e.g., VSVs) containing a CDV H polypeptide provided herein and/or a CDV F polypeptide provided herein. For example, a recombinant virus (e.g., VSV) can be designed to include (a) a CDV H polypeptide provided herein and a wild-type CDV F polypeptide, (b) a wild-type CDV H polypeptide and a CDV F polypeptide provided herein, or (c) a CDV H polypeptide provided herein and a CDV F polypeptide provided herein. In some cases, a recombinant virus (e.g., VSV) can be designed to include a CDV H polypeptide having CDV H 5804 and a CDV F polypeptide having CDV F 22458/16.

This document also provides nucleic acid molecules encoding a CDV H polypeptide provided herein and/or nucleic acid molecules encoding a CDV F polypeptide provided herein. For example, a nucleic acid molecule (e.g., a vector) can be designed to encode a CDV H polypeptide provided herein and/or a CDV F polypeptide provided herein.

This document provides methods and materials related to VSVs. For example, this document provides replication-competent VSVs, nucleic acid molecules encoding replication-competent VSVs, methods for making replication-competent VSVs, and methods for using replication-competent VSVs to treat cancer or infectious diseases.

As described herein, a VSV can be designed to have a nucleic acid molecule that encodes a VSV N polypeptide, a VSV P polypeptide, a VSV M polypeptide, a CDV F polypeptide (e.g., a CDV F polypeptide provided herein), a CDV H polypeptide (e.g., a CDV H polypeptide provided herein), and a VSV L polypeptide, and does not encode a functional VSV G polypeptide. It will be appreciated that the sequences described herein with respect to a VSV are incorporated into a plasmid coding for the positive sense cDNA of the viral genome allowing generation of the negative sense genome of VSVs. Thus, it will be appreciated that a nucleic acid sequence that encodes a VSV polypeptide, for example, can refer to an RNA sequence that is the template for the positive sense transcript that encodes (e.g., via direct translation) that polypeptide.

The nucleic acid encoding the CDV F polypeptide and the CDV H polypeptide can be positioned at any location within the VSV genome. In some cases, the nucleic acid encoding the CDV F polypeptide and the CDV H polypeptide can be positioned downstream of the nucleic acid encoding the VSV M polypeptide. For example, nucleic acid encoding a CDV F polypeptide and nucleic acid encoding a CDV H polypeptide can be positioned between nucleic acid encoding a VSV M polypeptide and nucleic acid encoding a VSV L polypeptide.

Any appropriate nucleic acid encoding a CDV F polypeptide can be inserted into the genome of a VSV. For example, nucleic acid encoding a wild-type CDV F polypeptide or a CDV F polypeptide provided herein can be inserted into the genome of a VSV.

Any appropriate nucleic acid encoding a CDV H polypeptide can be inserted into the genome of a VSV. For example, nucleic acid encoding a wild-type H polypeptide or a H polypeptide provided herein can be inserted into the genome of a VSV. In some cases, nucleic acid encoding a CDV H polypeptide that lacks specificity for SLAMF1 and/or Nectin-4 can be inserted into the genome of a VSV. For example, nucleic acid encoding a CDV H polypeptide having one or more mutations set forth in Table 2 can be inserted into the genome of a VSV. In some cases, a VSV/CDV hybrid provided herein can be designed to have a preselected tropism. For example, CDV F and/or H polypeptides having knocked out specificity for SLAMF1 and/or Nectin-4 can be used such that a scFv or polypeptide ligand can be attached to, for example, the C-terminus of the CDV H polypeptide. In such cases, scFv or polypeptide ligand can determine the tropism of a VSV/CDV hybrid. Examples of scFvs that can be used to direct VSV/CDV hybrids to cellular receptors (e.g., tumor associated cellular receptors) include, without limitation, anti-EGFR, anti-CD46, anti-αFR, anti-PSMA, anti-HER-2, anti-CD19, anti-CD20, or anti-CD38 scFvs. Examples of polypeptide ligands that can be used to direct VSV/CDV hybrids include, without limitation, urokinase plasminogen activator uPA polypeptides, cytokines such as IL-13, single chain T cell receptors (scTCRs), echistatin polypeptides, and integrin binding polypeptides.

In some cases, the nucleic acid molecule of VSV provided herein can encode an IFN polypeptide, a fluorescent polypeptide (e.g., a GFP polypeptide), a NIS polypeptide, a therapeutic polypeptide, an innate immunity antagonizing polypeptide, a tumor antigen, or a combination thereof. Nucleic acid encoding an IFN polypeptide can be positioned downstream of nucleic acid encoding a VSV M polypeptide. For example, nucleic acid encoding an IFN polypeptide can be positioned between nucleic acid encoding a VSV M polypeptide and nucleic acid encoding a CDV F polypeptide or nucleic acid encoding a CDV H polypeptide. Such a position can allow the viruses to express an amount of IFN polypeptide that is effective to activate anti-viral innate immune responses in non-cancerous tissues, and thus alleviate potential viral toxicity, without impeding efficient viral replication in cancer cells.

Any appropriate nucleic acid encoding an IFN polypeptide can be inserted into the genome of a VSV. For example, nucleic acid encoding an IFN beta polypeptide can be inserted into the genome of a VSV. Examples of nucleic acid encoding IFN beta polypeptides that can be inserted into the genome of a VSV include, without limitation, nucleic acid encoding a human IFN beta polypeptide of the nucleic acid sequence set forth in GenBank® Accession No. NM_002176.2 (GI No. 50593016), nucleic acid encoding a mouse IFN beta polypeptide of the nucleic acid sequence set forth in GenBank® Accession Nos. NM_010510.1 (GI No. 6754303), BC119395.1 (GI No. 111601321), or BC119397.1 (GI No. 111601034), and nucleic acid encoding a rat IFN beta polypeptide of the nucleic acid sequence set forth in GenBank® Accession No. NM_019127.1 (GI No. 9506800).

Nucleic acid encoding a NIS polypeptide can be positioned downstream of nucleic acid encoding a CDV F polypeptide or nucleic acid encoding a CDV H polypeptide. For example, nucleic acid encoding a NIS polypeptide can be positioned between nucleic acid encoding a CDV F or H polypeptide and nucleic acid encoding a VSV L polypeptide. Such a position of can allow the viruses to express an amount of NIS polypeptide that (a) is effective to allow selective accumulation of iodide in infected cells, thereby allowing both imaging of viral distribution using radioisotopes and radiotherapy targeted to infected cancer cells, and (b) is not so high as to be toxic to infected cells.

Any appropriate nucleic acid encoding a NIS polypeptide can be inserted into the genome of a VSV. For example, nucleic acid encoding a human NIS polypeptide can be inserted into the genome of a VSV. Examples of nucleic acid encoding NIS polypeptides that can be inserted into the genome of a VSV include, without limitation, nucleic acid encoding a human NIS polypeptide of the nucleic acid sequence set forth in GenBank® Accession Nos. NM_000453.2 (GI No. 164663746), BC105049.1 (GI No. 85397913), or BC105047.1 (GI No. 85397519), nucleic acid encoding a mouse NIS polypeptide of the nucleic acid sequence set forth in GenBank® Accession Nos. NM_053248.2 (GI No. 162138896), AF380353.1 (GI No. 14290144), or AF235001.1 (GI No. 12642413), nucleic acid encoding a chimpanzee NIS polypeptide of the nucleic acid sequence set forth in GenBank® Accession No. XM_524154 (GI No. 114676080), nucleic acid encoding a dog NIS polypeptide of the nucleic acid sequence set forth in GenBank® Accession No. XM_541946 (GI No. 73986161), nucleic acid encoding a cow NIS polypeptide of the nucleic acid sequence set forth in GenBank® Accession No. XM_581578 (GI No. 297466916), nucleic acid encoding a pig NIS polypeptide of the nucleic acid sequence set forth in GenBank® Accession No. NM_214410 (GI No. 47523871), and nucleic acid encoding a rat NIS polypeptide of the nucleic acid sequence set forth in GenBank® Accession No. NM_052983 (GI No. 158138504).

The nucleic acid sequences of a VSV provided herein that encode a VSV N polypeptide, a VSV P polypeptide, a VSV M polypeptide, and a VSV L polypeptide can be from a VSV Indiana strain as set forth in GenBank® Accession Nos. NC_001560 (GI No. 9627229) or can be from a VSV New Jersey strain.

In one aspect, this document provides VSVs containing a nucleic acid molecule (e.g., an RNA molecule) having (e.g., in a 3′ to 5′ direction) a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a CDV F polypeptide, a nucleic acid sequence that is a template for a positive sense transcript encoding a CDV H polypeptide, and a nucleic acid sequence that is a template for a positive sense transcript encoding a VSV L polypeptide while lacking a nucleic acid sequence that is a template for a positive sense transcript encoding a functional VSV G polypeptide. Such VSVs can infect cells (e.g., cancer cells) and be replication-competent.

Any appropriate method can be used to insert nucleic acid (e.g., nucleic acid encoding a CDV F polypeptide, nucleic acid encoding a CDV H polypeptide, nucleic acid encoding an IFN polypeptide, and/or nucleic acid encoding a NIS polypeptide) into the genome of a VSV. For example, the methods described elsewhere (Schnell et. al., PNAS, 93:11359-11365 (1996), Obuchi et al., J. Virol., 77(16):8843-56 (2003)); Goel et al., Blood, 110(7):2342-50 (2007)); and Kelly et al., J. Virol., 84(3):1550-62 (2010)) can be used to insert nucleic acid into the genome of a VSV. Any appropriate method can be used to identify VSVs containing a nucleic acid molecule described herein. Such methods include, without limitation, PCR and nucleic acid hybridization techniques such as Northern and Southern analysis. In some cases, immunohistochemistry and biochemical techniques can be used to determine if a VSV contains a particular nucleic acid molecule by detecting the expression of a polypeptide encoded by that particular nucleic acid molecule.

In another aspect, this document provides nucleic acid molecules that encode a VSV N polypeptide, a VSV P polypeptide, a VSV M polypeptide, a CDV F polypeptide, a CDV H polypeptide, and a VSV L polypeptide, while lacking the ability to encode a functional VSV G polypeptide. For example, a nucleic acid molecule provided herein can be a single nucleic acid molecule that includes a nucleic acid sequence that encodes a VSV N polypeptide, a nucleic acid sequence that encodes a VSV P polypeptide, a nucleic acid sequence that encodes a VSV M polypeptide, a nucleic acid sequence that encodes a CDV F polypeptide, a nucleic acid sequence that encodes a CDV H polypeptide, and a nucleic acid sequence that encodes a VSV L polypeptide, while lacking a nucleic acid sequence that encodes a functional VSV G polypeptide.

In another aspect, this document provides nucleic acid molecules that encode a VSV N polypeptide, a VSV P polypeptide, a VSV M polypeptide, an IFN polypeptide, a CDV F polypeptide, a CDV H polypeptide, a NIS polypeptide, and a VSV L polypeptide, while lacking the ability to encode a functional VSV G polypeptide. For example, a nucleic acid molecule provided herein can be a single nucleic acid molecule that includes a nucleic acid sequence that encodes a VSV N polypeptide, a nucleic acid sequence that encodes a VSV P polypeptide, a nucleic acid sequence that encodes a VSV M polypeptide, a nucleic acid sequence that encodes an IFN polypeptide, a nucleic acid sequence that encodes a CDV F polypeptide, a nucleic acid sequence that encodes a CDV H polypeptide, a nucleic acid sequence that encodes a NIS polypeptide, and a nucleic acid sequence that encodes a VSV L polypeptide, while lacking the ability to encode a functional VSV G polypeptide.

The term “nucleic acid” as used herein encompasses both RNA (e.g., viral RNA) and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

This document also provides method for treating cancer (e.g., to reduce tumor size, inhibit tumor growth, or reduce the number of viable tumor cells), methods for inducing host immunity against cancer, and methods for treating an infectious disease such as HIV or measles. For example, a recombinant virus (e.g., a VSV) provided herein can be administered to a mammal having cancer to reduce tumor size, to inhibit cancer cell or tumor growth, to reduce the number of viable cancer cells within the mammal, and/or to induce host immunogeneic responses against a tumor. A recombinant virus (e.g., a VSV) provided herein can be propagated in host cells in order to increase the available number of copies of that virus, typically by at least 2-fold (e.g., by 5- to 10-fold, by 50- to 100-fold, by 500- to 1,000-fold, or even by as much as 5,000- to 10,000-fold). In some cases, a recombinant virus (e.g., a VSV) provided herein can be expanded until a desired concentration is obtained in standard cell culture media (e.g., DMEM or RPMI-1640 supplemented with 5-10% fetal bovine serum at 37° C. in 5% CO₂). A viral titer typically is assayed by inoculating cells (e.g., Vero cells) in culture.

Recombinant viruses (e.g., VSVs) provided herein can be administered to a cancer patient by, for example, direct injection into a group of cancer cells (e.g., a tumor) or intravenous delivery to cancer cells. A recombinant virus (e.g., a VSV) provided herein can be used to treat different types of cancer including, without limitation, myeloma (e.g., multiple myeloma), melanoma, glioma, lymphoma, mesothelioma, and cancers of the lung, brain, stomach, colon, rectum, kidney, prostate, ovary, breast, pancreas, liver, and head and neck.

Recombinant viruses (e.g., VSVs) provided herein can be administered to a patient in a biologically compatible solution or a pharmaceutically acceptable delivery vehicle, by administration either directly into a group of cancer cells (e.g., intratumorally) or systemically (e.g., intravenously). Suitable pharmaceutical formulations depend in part upon the use and the route of entry, e.g., transdermal or by injection. Such forms should not prevent the composition or formulation from reaching a target cell (i.e., a cell to which the virus is desired to be delivered to) or from exerting its effect For example, pharmacological compositions injected into the blood stream should be soluble.

While dosages administered will vary from patient to patient (e.g., depending upon the size of a tumor), an effective dose can be determined by setting as a lower limit the concentration of virus proven to be safe and escalating to higher doses of up to 10¹² pfu, while monitoring for a reduction in cancer cell growth along with the presence of any deleterious side effects. A therapeutically effective dose typically provides at least a 10% reduction in the number of cancer cells or in tumor size. Escalating dose studies can be used to obtain a desired effect for a given viral treatment (see, e.g., Nies and Spielberg, “Principles of Therapeutics,” In Goodman & Gilman's The Pharmacological Basis of Therapeutics, eds. Hardman, et al., McGraw-Hill, N Y, 1996, pp 43-62).

Recombinant viruses (e.g., VSVs) provided herein can be delivered in a dose ranging from, for example, about 10³ pfu to about 10¹² pfu (e.g., about 10⁵ pfu to about 10¹² pfu, about 10⁶ pfu to about 10¹¹ pfu, or about 10⁶ pfu to about 10¹⁰ pfu). A therapeutically effective dose can be provided in repeated doses. Repeat dosing is appropriate in cases in which observations of clinical symptoms or tumor size or monitoring assays indicate either that a group of cancer cells or tumor has stopped shrinking or that the degree of viral activity is declining while the tumor is still present. Repeat doses can be administered by the same route as initially used or by another route. A therapeutically effective dose can be delivered in several discrete doses (e.g., days or weeks apart) and in one embodiment, one to about twelve doses are provided. Alternatively, a therapeutically effective dose of recombinant viruses (e.g., VSVs) provided herein can be delivered by a sustained release formulation. In some cases, a recombinant virus (e.g., a VSV) provided herein can be delivered in combination with pharmacological agents that facilitate viral replication and spread within cancer cells or agents that protect non-cancer cells from viral toxicity. Examples of such agents are described elsewhere (Alvarez-Breckenridge et al., Chem. Rev., 109(7):3125-40 (2009)).

Recombinant viruses (e.g., VSVs) provided herein can be administered using a device for providing sustained release. A formulation for sustained release of recombinant viruses (e.g., VSVs) provided herein can include, for example, a polymeric excipient (e.g., a swellable or non-swellable gel, or collagen). A therapeutically effective dose of recombinant viruses (e.g., VSVs) provided herein can be provided within a polymeric excipient, wherein the excipient/virus composition is implanted at a site of cancer cells (e.g., in proximity to or within a tumor). The action of body fluids gradually dissolves the excipient and continuously releases the effective dose of virus over a period of time. Alternatively, a sustained release device can contain a series of alternating active and spacer layers. Each active layer of such a device typically contains a dose of virus embedded in excipient, while each spacer layer contains only excipient or low concentrations of virus (i.e., lower than the effective dose). As each successive layer of the device dissolves, pulsed doses of virus are delivered. The size/formulation of the spacer layers determines the time interval between doses and is optimized according to the therapeutic regimen being used.

In some cases, recombinant viruses (e.g., VSVs) provided herein can be directly administered. For example, a virus can be injected directly into a tumor (e.g., a breast cancer tumor) that is palpable through the skin. Ultrasound guidance also can be used in such a method. Alternatively, direct administration of a virus can be achieved via a catheter line or other medical access device, and can be used in conjunction with an imaging system to localize a group of cancer cells. By this method, an implantable dosing device typically is placed in proximity to a group of cancer cells using a guidewire inserted into the medical access device. An effective dose of a recombinant virus (e.g., a VSV) provided herein can be directly administered to a group of cancer cells that is visible in an exposed surgical field.

In some cases, recombinant viruses (e.g., VSVs) provided herein can be delivered systemically. For example, systemic delivery can be achieved intravenously via injection or via an intravenous delivery device designed for administration of multiple doses of a medicament. Such devices include, but are not limited to, winged infusion needles, peripheral intravenous catheters, midline catheters, peripherally inserted central catheters, and surgically placed catheters or ports.

The course of therapy with a recombinant virus (e.g., a VSV) provided herein can be monitored by evaluating changes in clinical symptoms or by direct monitoring of the number of cancer cells or size of a tumor. For a solid tumor, the effectiveness of virus treatment can be assessed by measuring the size or weight of the tumor before and after treatment. Tumor size can be measured either directly (e.g., using calipers), or by using imaging techniques (e.g., X-ray, magnetic resonance imaging, or computerized tomography) or from the assessment of non-imaging optical data (e.g., spectral data). For a group of cancer cells (e.g., leukemia cells), the effectiveness of viral treatment can be determined by measuring the absolute number of leukemia cells in the circulation of a patient before and after treatment. The effectiveness of viral treatment also can be assessed by monitoring the levels of a cancer specific antigen. Cancer specific antigens include, for example, carcinoembryonic antigen (CEA), prostate specific antigen (PSA), prostatic acid phosphatase (PAP), CA 125, alpha-fetoprotein (AFP), carbohydrate antigen 15-3, and carbohydrate antigen 19-4.

The invention will be further described in the following examples, which do not limit the scope of the invention described in the claims.

### EXAMPLES

### Example 1—CDV F and H polypeptides and recombinant viruses

### Cell Lines

Vero African green monkey kidney cells (Vero; American type culture collection [ATCC], Cat. #CCL-81) and their derivatives (either expressing Nectin-4 (Noyce et al., Virology, 436(1):210-20 (2013)), SLAMF1 (Tatsuo et al., Nature, 406(6798):893-7 (2000)), dog SLAMF1 (von Messling et al., J. Virol., 77(23):12579-91 (2003)), or a membrane-anchored single-chain antibody specific for a hexahistidine peptide (SEQ ID NO: 21) (Nakamura et al., Nat. Biotechnol., 23(2):209-14 (2005)) were kept in Dulbecco's modified Eagle's medium (DMEM) (GE Healthcare Life Sciences, Cat. #SH30022.01) supplemented with 5% (vol./vol.) heat-inactivated fetal bovine serum (FBS, Gibco) and 0.5 mg/mL of Geneticin (G418; Coming) for Vero/NECTIN-4 and Vero/SLAMF1 or 1 mg/mL Zeocin (ThermoFisher) for Vero/dogSLAMF1. Human kidney epithelial cells (HEK293T) cells, obtained from Dr. Cosset (Universitd de Lyon), baby hamster kidney cells (BHK, ATCC, Cat. #CCL-10), human glioblastoma U-87 MG cells ((ATCC, Cat. #HTB-14), and SKOV3ip.1 human ovarian tumor cells were maintained in DMEM plus 10% FBS. Chinese hamster ovary cells (CHO) cells, CHO-CD46, and CHO-EGFR (Nakamura et al., Nat. Biotechnol., 22(3):331-6 (2004)), CHO-SLAMF1 (Tatsuo et al., Nature, 406(6798):893-7 (2000)), CHO-dogSLAMF1 (Seki et al., J. Virol., 77(18):9943-50 (2003)), CHO-NECTIN4 (Liu et al., J. Virol., 88(4):2195-204 (2014)), CHO-CD38 (Peng et al. Blood, 101:2557-62 (2003), CHO-HER2/neu (Hasegawa et al., J. Virol., 81(23): 13149-57 (2007), Burkitt's B cell lymphoma Ramos (ATCC, Cat. #CRL-1596), and Raji cells (ATCC, Cat. #CCL-86) were cultured in Roswell Park Memorial Institute (RPMI) 1640 medium (Corning Inc., Cat. #10-040-CV, Coming, NY, United States) as described elsewhere.

Plasmids and Construction of Full-Genome rMeV

To generate CDV 22458/16 expression plasmids, total RNA was extracted from CDV 22458/16 isolate-infected Vero/dog SLAMF1 cells (passage 1) using RNeasy Mini Kit (Qiagen, Hilden, Germany). Both CDV-H and CDV-F genes were reverse transcribed with SuperScript III Reverse Transcriptase (Thermo Fisher Scientific, Cat.#11752050) and amplified by PCR with the following primers:

CDVH7050(+):
(SEQ ID NO: 22)
AGAAAACTTAGGGCTCAGGTAGTCC
CDVH8949(−):
(SEQ ID NO: 23)
TCGTCTGTAAGGGATTTCTCACC
CDVF4857(+):
(SEQ ID NO: 24)
AGGACATAGCAAGCCAACAGG
CDVH7050(−):
(SEQ ID NO: 25)
GGACTACCTGAGCCCTAAGTTTTCT

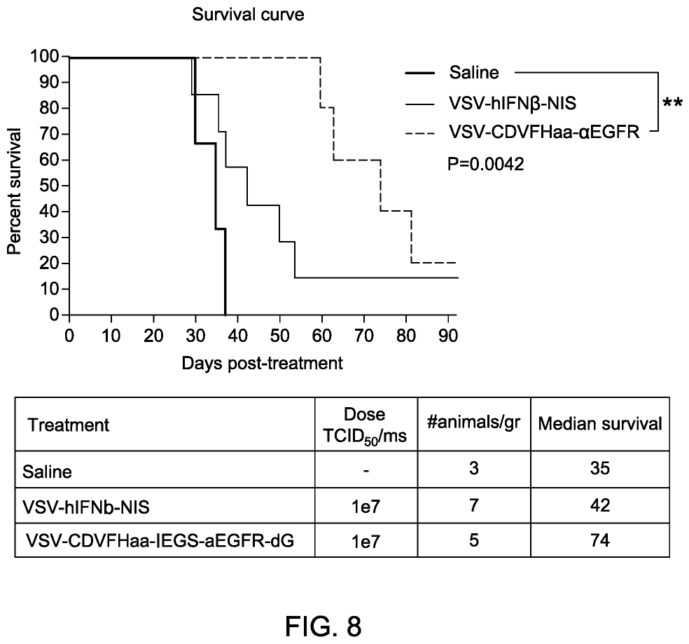

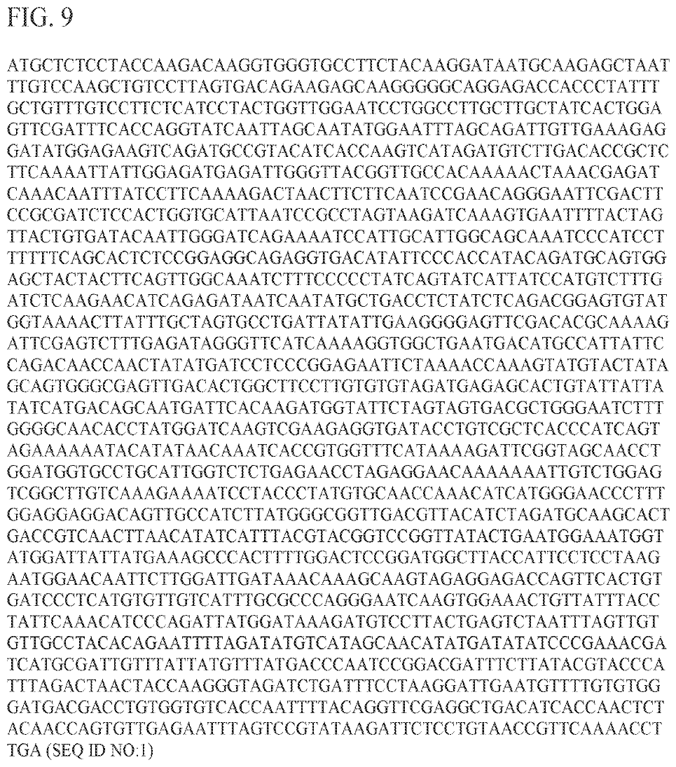

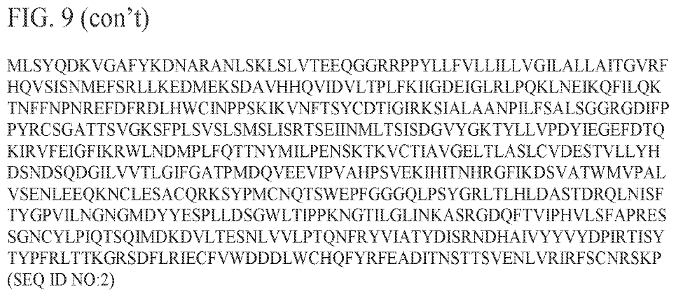

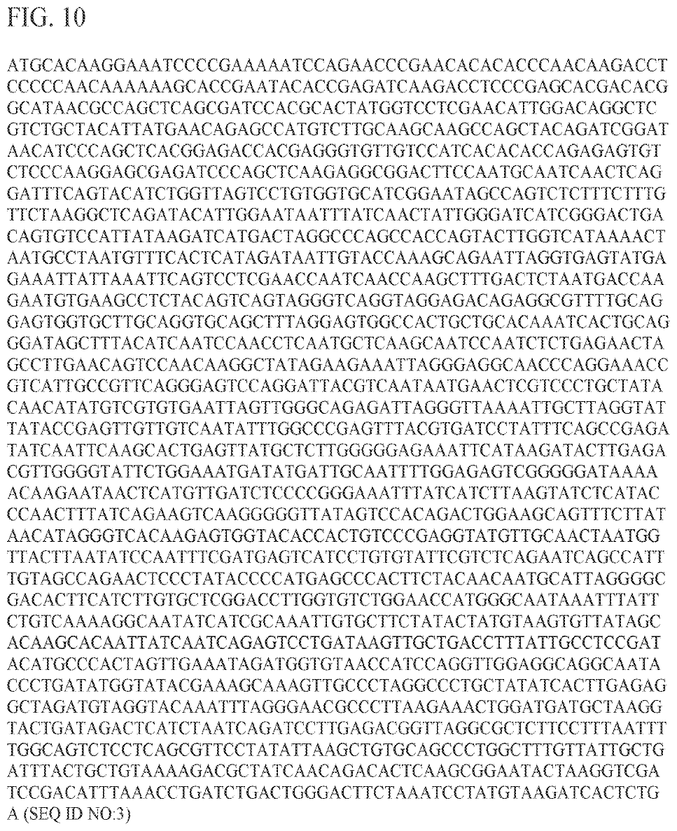

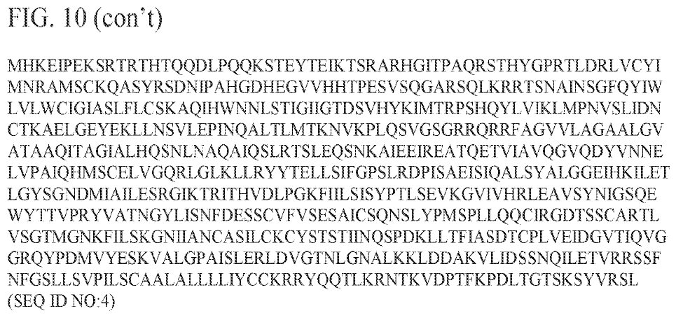

PCR products were sequenced directly by Sanger (Genewiz, Plainfield NJ, USA) and cloned into pJET1.2 vector (Thermo Fisher). CDV H open reading frame (FIG. 8) was next PCR amplified with a forward primer (5′-CCG GTA GTT AAT TAA AAC TTA GGG TGC AAG ATC ATC GAT AAT GCT CTC CTA CCA AGA TAA GGT G-3′ (SEQ ID NO: 26)) and a reverse primer (5′-CTA TTT CAC ACT AGT GGG TAT GCC TGA TGT CTG GGT GACATC ATG TGA TTG GTT CAC TAG CAG CCT CAA GGT TTT GAA CGG TTA CAG GAG-3′ (SEQ ID NO: 27)) and cloned into the PacI and SpeI (New England Biolabs, Iswich MA, USA) restricted pCG vector (Cathomen et al., J. Virol., 72(2):1224-34 (1998)) using the In Fusion HD kit (Takara, Shinagawa, Tokyo, Japan). The primers provided the PacI and SpeI restriction sites (underlined, respectively) as well as the MeV-H untranslated region of MeV-H (italic). Similarly, a CDV F open reading frame (FIG. 9; amino acid residues 136-662 of SEQ ID NO:4) was cloned into HpaI/SpeI restricted pCG-CDV-F plasmid (von Messling et al., J. Virol., 75(14):6418-27 (2001)). The resulting plasmid pCG-CDV F 22458/16 possesses the MeV-F untranslated region and the MeV-F signal peptide.

Expression plasmids for CDV H/F Onderstepoort vaccine and 5804 isolate (von Messling et al., J. Virol., 75(14):6418-27 (2001)), as well as the MeV Nse are described elsewhere (Cathomen et al., J. Virol., 72(2):1224-34 (1998)). Retargeted versions of the H protein were generated by inserting the homologous PacI/SfiI-digested PCR product into the pTNH6 vectors (Nakamura et al., Nat. Biotechnol., 23(2):209-14 (2005); and Nakamura et al., Nat. Biotechnol., 22(3):331-6 (2004)). Site-directed mutagenesis (QuickChange Site-Directed Mutagenesis Kit, Agilent Technologies, Santa Clara CA, USA) was used to ablate tropism in H as well as to remove a SpeI site in CDV-F and to introduce truncations in the cytoplasmic tails.

Envelope-exchange rMeVs were produced by shuttling the PacI/SpeI and NarI/PacI region of the corresponding expression plasmids. Rescue of rMeVs was carried out employing the START system (Nakamura et al., Nat. Biotechnol., 23(2):209-14 (2005)).

### Protein Expression

Cells were transfected using Fugene HD (PROMEGA, Fitchburg WI, USA) or TransIT-LT1 transfection reagent (Mirus Bio LLC, Madison WI, USA). For quantitative fusion assay, the dual-split reporter system (Kondo et al., J. Biol. Chem., 285(19):14681-8 (2010); and Ishikawa et al., Protein Eng. Des. Sel., 25(12):813-20 (2012)) was used as described elsewhere (Muñoz-Alia et al., Viruses, 11(8), pii: E688, doi: 0.3390/v11080688 (2019)), using BHK cells as effector cells. For semiquantitative assessment of fusion, Vero cells and derivates were transfected with 1 μg each of H and F expression plasmids and stained 1 day later with Hema-Quik (Thermo Fisher Scientific, Cat. #123-745). Images were obtained with a microscope (Eclipse Ti-S; Nikon) at 4× magnification. Alternatively, a GFP expression plasmid was included for additional visualization of syncytia formation. For assessment of the level of H polypeptide, transfected cells were analyzed by flow cytometry or CELISA using a 6×His tag monoclonal antibody (Miltenyi Biotec, Cat. #130-120-787 or Thermo Fisher Scientific, Cat. #MA1-135), as described elsewhere (Muñoz-Alia et al., Viruses, 11(8), pii: E688, doi: 10.3390/v11080688 (2019); and Saw et al., Methods, 90:68-75 (2015)). For total protein expression by flow cytometry, cells were treated with eBioscience Intracellular Fixation & Permeabilization buffer (Thermofisher, Cat. #88-8823-88).

### Virus Protein Content

Virus preparations were heated in the presence of dithiothreitol, fractionated into 4-12% Bis-Tris polyacrylamide gel, and transferred to polyvinylidene fluoride membranes. Blots were analyzed with anti-MeV-Hcyt (Cathomen et al., J. Virol., 72(2):1224-34 (1998)), anti-MeV-N(Toth et al., J. Virol., 83(2):961-8 (2009)), or anti-His tag (Genscript, Piscataway NJ, USA, Cat. #A01857-40) antibodies and probed with a conjugated secondary rabbit antibody (ThermoFisher, Cat. #31642). The blots were incubated with SuperSignal Wester Pico chemiluminescent substrate (ThermoFisher) and analyzed with a ChemiDoc Imaging Sytem (Bio-Rad).

### Neutralization Assays

A fluorescence focus reduction neutralization assay was used as described elsewhere (Munoz-Alia et al., J. Virol., 91(11): e00209-17 (2017)). The polyclonal Anti-Canine Distemper Virus, Lederle Avirulent (antiserum, Ferret), was obtained through BEI resources (NR-4025). The human sera used was pooled from 60 to 80 donors who were specifically blood type AB (Valley Biomedical Products & Services, Inc, Cat. #HS1017, Lot #C80553).

### Results

### Engineering a Fusogenic CDV H/F Complex

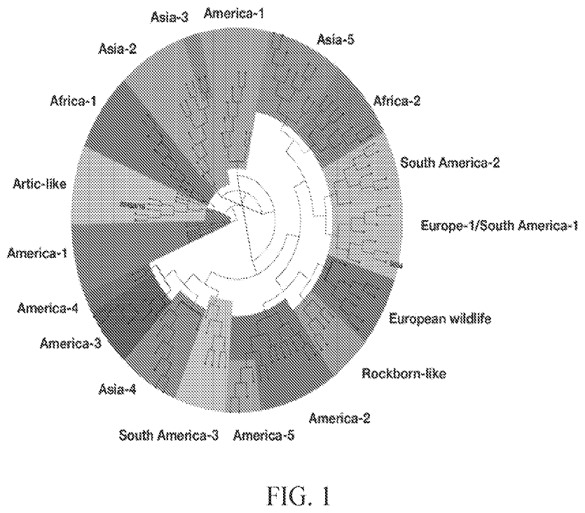

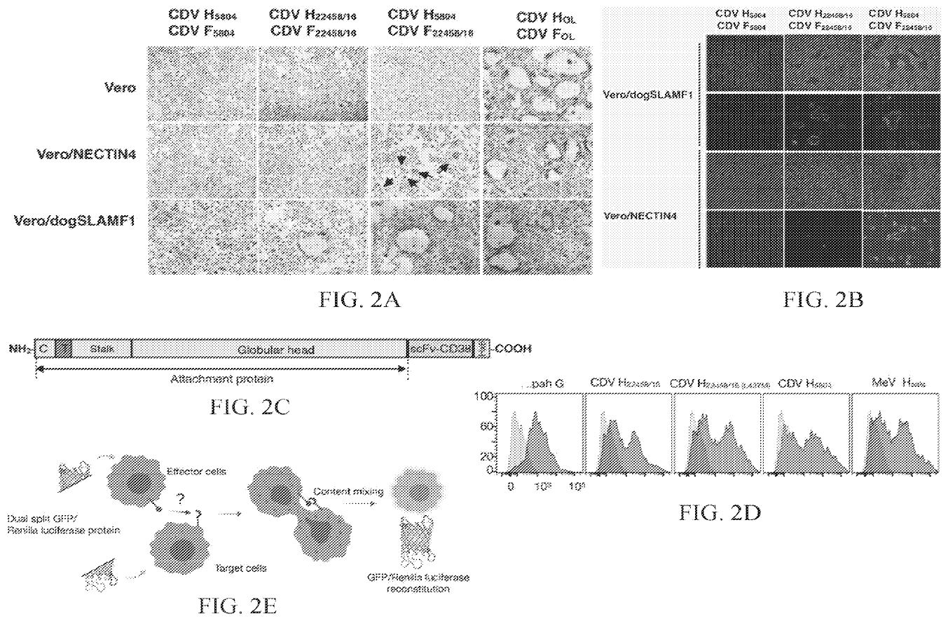

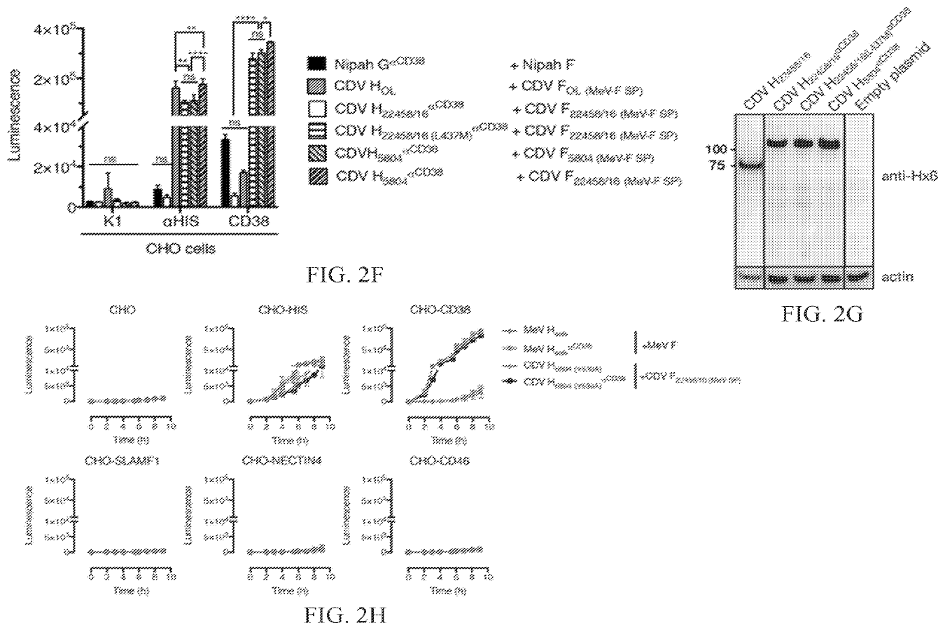

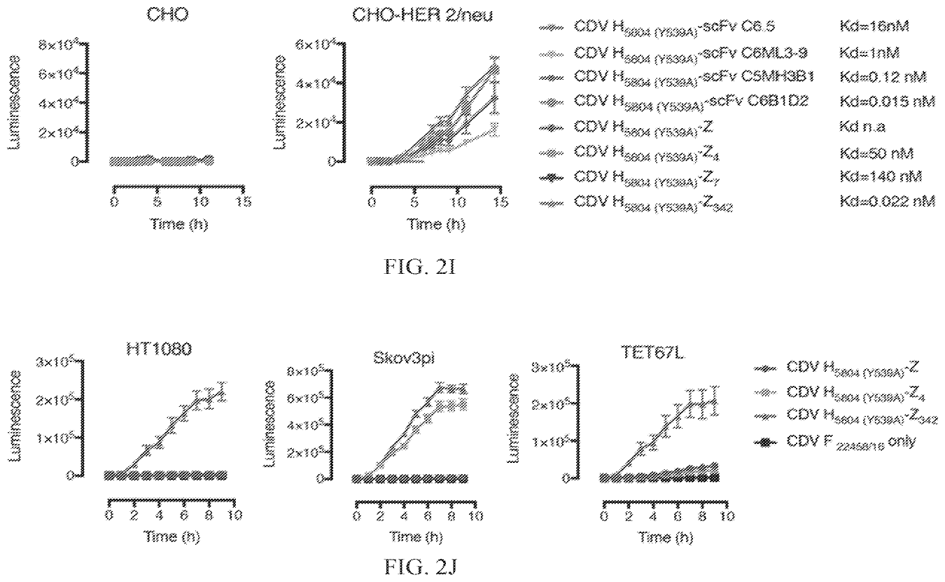

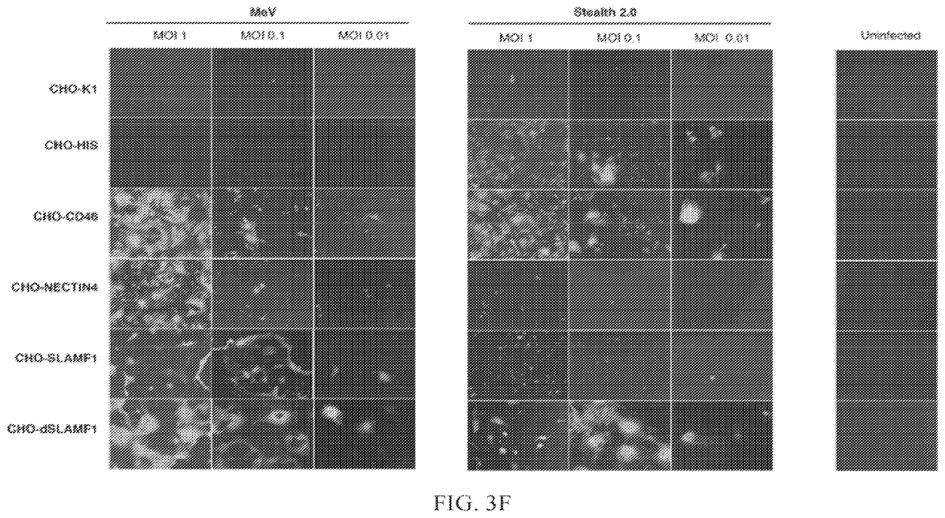

CDV envelope glycoproteins share a 36% (H polypeptide) and 66% (F polypeptide) amino acid homology with those of MeV. The open reading frame for the H and F polypeptides were obtained from a first-passage wild-type CDV isolate SPA.Madrid/22458/16 (CDV 22458/16) from post-mortem tissues from moribund dog (SPA.Madrid/22458/16). Maximum-likelihood phylogenetic analysis of fill-length hemagglutinin genes showed that CDV H polypeptide of 22458/16 grouped within the artic clade (FIG. 1). Co-transfection of H/F complexes from 22458/16 revealed fusion activity only when the SLAMF1 receptor was present (FIG. 2A). A lack of fusion activity on Nectin-4-expressing cells was observed. Co-expression of a heterotypic wild type CDV H polypeptide of 5804 with CDV F polypeptide of 22458/16 resulted in noticeable syncytia formation in Vero cells expressing Nectin-4. On the other hand, H/F complexes from the large-plaque forming variant of the Onderstepoort vaccine strain resulted in large syncytia formation regardless of the expression of Nectin-4 and SLAMF1.

Using a reporter gene to more accurately identify syncytia formation, the above fusogenic phenotype was confirmed for the heterotypic wild-type combination CDV H₅₈₀₄ polypeptide and CDV F_{22458/16} polypeptide (FIG. 2B). Although the lack of syncytia formation for the homotypic H/F₅₈₀₄ could be attribute to the presence of the native 135 amino acid-long signal peptide, the distinct fusogenic phenotype observed for CDV H/F_{22458/16}, which induced cell-to-cell fusion exclusively in a SLAMF1-dependent manner, was studied further. The clone used for CDV H_{22458/16} polypeptide was found to contain an amino acid change with regard to the consensus sequence (M437L). This clone was used to create a clone encoding a CDV H_{22458/16} polypeptide with position 437 changed from a leucine to a methionine.

### Tropism Expansion of CDV Fusion Apparatus

Since co-transfection of wild-type CDV H/F complexes, but not the Ondertepoort vaccine-derived H/F, resulted in syncytia formation in a specific receptor-dependent manner, the following was performed to determine whether receptor usage could be expanded to alternative receptors. As a target receptor, CD38 was chosen and for this purpose a CD38-specific scFv was displayed at the carboxy-terminal domain of the attachment protein (FIG. 2C). For comparison purposes, the analysis included the MeV H polypeptide as well as the Nipah G polypeptide. Results presented in FIG. 2D demonstrate that the different constructs were expressed on the surface at comparable levels. Of note, the L437M substitution of CDV H_{22458/16} did not seem to affect cell surface expression. Next, fusion proficiency was compared in a quantitative manner using the self-associating split luciferase assay described elsewhere (Kondo et al., J. Biol. Chem., 285:14681-14688 (2010); and Ishikawa et al., Protein Eng. Des. Sel., 25:813-820 (2012)). In this assay (FIG. 2E), effector cells were transfected with expression plasmids for the H/F complex and one half of the dual split GFP/Renilla luciferase protein (DSP1-7). Similarly, target cells expressing the relevant receptor were transfected with the other half (DSP8-12). Upon content mixing, the otherwise nonfunctional halves of the GFP/Renilla luciferase protein associate, and the activity measured. The results of this experiment with effector cells expressing different H or G/F complexes are shown in FIG. 2F. Whereas no fusion activity was observed when using the parental CHO cell line, fusion activity was evident on CHO cells engineered to stably express either a HIS-specific scFv (CHO-HIS) or CD38 molecule. In general, activity levels were more evident when using CD38 targeting versus the pseudoreceptor system 6×His-anti-6×HIS scFv. These differences were more noticeable when Nipah G^{αCD38} was used. The fusion capacity of the former was significantly reduced in comparison with the heterotypic CDV H₅₈₀₄^{αCD38} polypeptide/CDV F_{22458/16} polypeptide or the homotypic H/F from the Onderstepoort strain. Strikingly, the L437M substitution on CDV H_{22458/16} polypeptide drastically changed the fusogenic phenotype of the polypeptide, even when no differences at the expression levels were observed (FIG. 2G). This new fusogenic capacity was now equivalent to that obtained with homotypic CDV H/F from 5804, only when the signal peptide of the CDV F₅₈₀₄ polypeptide was replaced by a shorter MeV F signal peptide. Nevertheless, the fusion levels for the heterotypic combination of CDV H₅₈₀₄ ^{αCD38} polypeptide/CDV F_{22458/16} polypeptide were superior and similar to those obtained by the large-plaque forming variant of the Onderstepoort. These results demonstrate not only that the CDV fusion apparatus can be engineered to use alternative receptors, but also that a hyperfusogenic phenotype can be obtained from a heterotypic combination of H/F complexes from different CDV strains in a receptor-dependent manner. This enhanced fusogenic capacity for the CDV H/F complex was achieved by shortening of the signal peptide of the CDV F polypeptide and by using a CDV polypeptide having M437.

### Receptor Targeting

The CDV H polypeptides described in the preceding paragraph could still use human Nectin-4 as a receptor. To disrupt a Nectin-4 interaction, nucleic acid encoding a CDV H polypeptide containing a Y539A mutation was produced. FIG. 2H shows that the CDV H^{αCD38} polypeptide including the Y539A point mutation (CDVH_{Y539A}^{αCD38}) lost fusion activity on human Nectin-4 cells, but it was still able to induce fusion on CHO-HIS (CDV HY_{539A}^{αCD388} and CDV H_{Y539A}) and CHO-CD38 (CDV H_{Y539A}^{αCD388}) cells. This fusion activity was comparable to that obtained by a fully-retargeted MeV H polypeptide (MeV Haals^{αCD38} and MeV Haals^{αCD38}). These results demonstrate that the CDV H/F complex can be efficiently retargeted to specific receptors.

### Impact of Ligand-Binding Affinity on CDV H/F Driven Cell-to-Cell Fusion

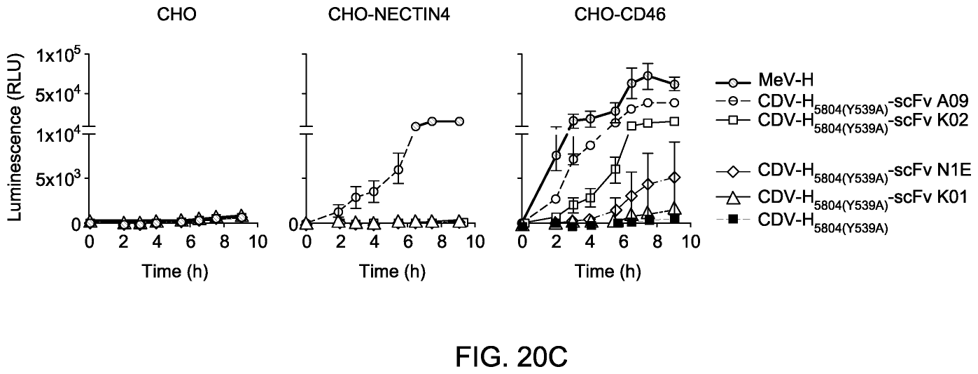

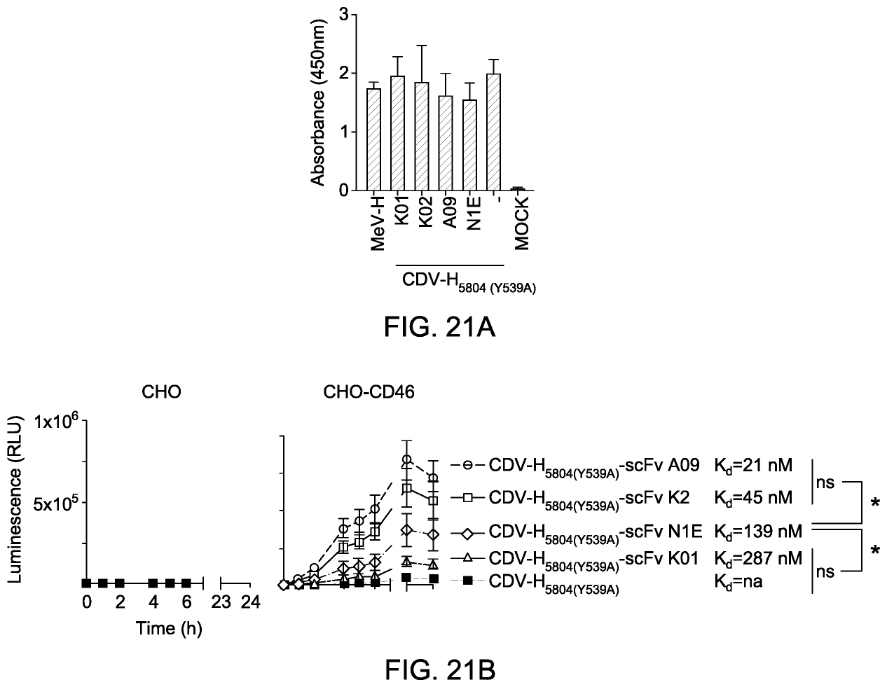

To assess whether differences in the binding affinity of the ligand displayed to the ectodomain of the CDV H polypeptide influenced fusogenicity, Her2/neu-specific scFv binders as well as affibodies molecules (Hasegawa et al. J. Virol., 81(23): 13149-57 (2007); Wikman et al. Protein Eng. Des. Sel., 17(5):455-62 (2004); and Orlova et al. Cancer Res., 66(8):4339-48 (2006)) were displayed. FIG. 21 shows that binding affinities higher than 1 nM were necessary to trigger fusion on CHO cells engineered to stably express Her2/neu molecules, but not in the parental cell line. This was true independently of the nature of the binder, whether it was in the form of a scFv or an affibody molecule. To investigate the relationship between the receptor density and binder affinity, the quantitative fusion assay was repeated with an array of cancer cell lines expressing different levels of Her2/neu molecules on the surface: HT1080 (1.2×10⁴), Sko3pi (1.5×10⁵), and TET67L (4.3×10³). Whereas display of the affibody with the highest binding affinity (Z342, 0.022 nM) enabled CDV H to trigger fusion in all cell lines tested regardless of the receptor density, Z4 (50 nM) enabled intercellular fusion only in Skov3pi cells, which expressed the highest receptor density. These results demonstrate that there is an interplay between the binder-affinity and the receptor density on the target cell: lower receptor densities being effect with higher binder affinities.

### CD46-Targeted CDV t F Complexes can Overcome Neutralization-Sensitivity of Oncolytic Measles Virus

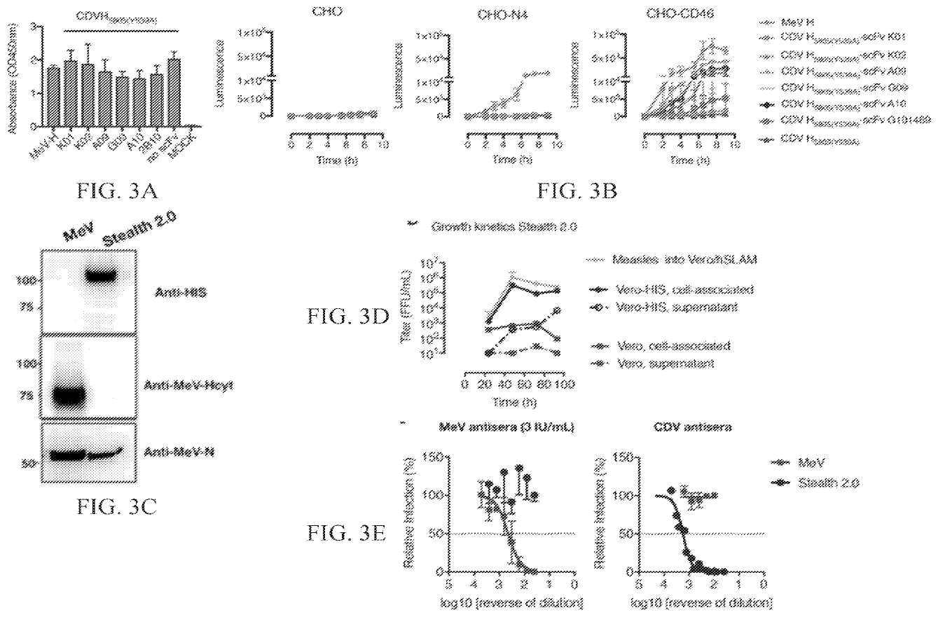

Different CD46-specific scFv binders were displayed on CDV H polypeptides in an attempt to obtain scFv-CDV H polypeptides supporting a similar fusion level to those of a MeV H Nse strain Cell surface expression levels were compared (FIG. 3A). A cellular enzyme-linked immunosorbent assay (CELISA) showed that both untargeted MeV H polypeptides and CDV H polypeptides as well as CD46-targeted CDV H polypeptides were similarly expressed at the cell surface. Next, quantitative fusion assays revealed that whereas only MeV H polypeptides resulted in fusion activity in CHO-Nectin-4 cells. all but the untargeted CDV H polypeptides produced fusion in CHO-CD46 cells (FIG. 3B). scFv binders A10, A09, G09. and K2 displayed on CDV H polypeptides induced fusion levels similar to those seen in MeV H polypeptides, whereas scFV G101469 and K01 were comparably lower CDV H-scFv A09/CDV F polypeptide were chosen to replace the existing MeV coat. This virus. referred to herein as Stealth 2.0, was rescued, and the H/F polypeptides were confirmed to be successfully displayed on the virions. Western Blot analysis confirmed that MeV H polypeptide was detected only in the case of MeV when an anti-MeV H polypeptide antibody against the cytoplasmic tail was used. On the contrary. CDV H-scFV A09 polypeptide was only detected when the membrane was probed with an anti 6×HIS-tag antibody (FIG. 3C). This same HIS-tag system allowed the replication of the Stealth 2.0 virus in Vero cells stably expressing the anti-HIS scFv but not in the parental Vero cell line (FIG. 3D). The replication kinetics was comparable to that obtained by MeV on Vero/hSLAM. Together, these results demonstrate that the measles virus envelope H and F polypeptides can be replaced by H and F polypeptides from CDV without a negative impact on virus replication.

The neutralization sensitivity of Stealth 2.0 was studied using pooled serum from 20-30 American donors. As control, CDV antisera was used. FIG. 3E shows that Stealth 2.0 was insensitive to the neutralization activity of MeV antisera On the contrary, the neutralization pattern of MeV was essentially the opposite, being neutralized by anti-measles antibodies, but not by anti-CDV antibodies.

The following was performed to confirm the virus tropism endowed by the new envelope since virus-entry could occurred in the absence of evident fusion. Virus-derived GFP autofluorescence was observed when CHO cells expressed the receptors CD46, Nectin-4, and either canine or human SLAMF1 (FIG. 3F). Conversely. GFP-autofluorescence driven by Stealth 2.0 was only observed in the case of anti-6×HIS scFv expression (CHO-HIS). CD46. and canine SLAMF1. These results demonstrate that a human CD46-tropic measles virus resistant to anti-measles virus antibody neutralization can be generated by using a CD46-targeted CDV H/F envelope.

### Stealth 2.0 Induces a Similar Anti-Tumor Effect as the Parental MeV

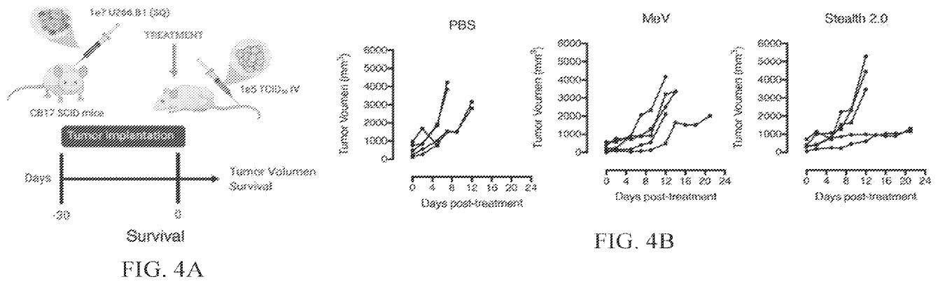

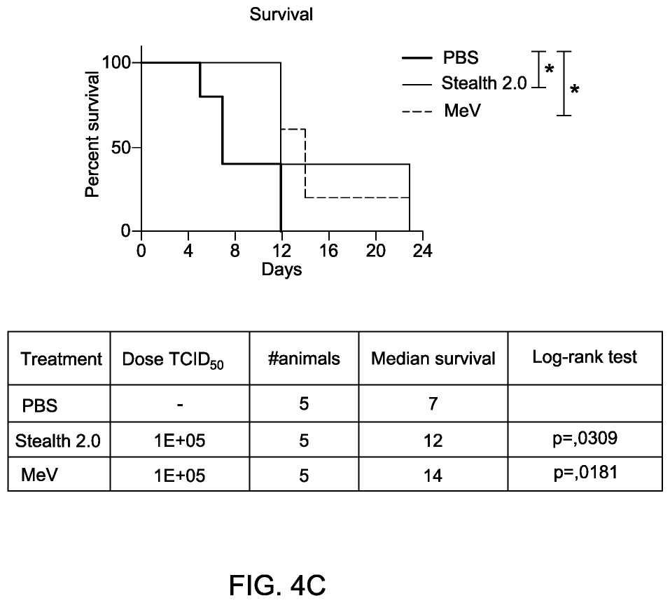

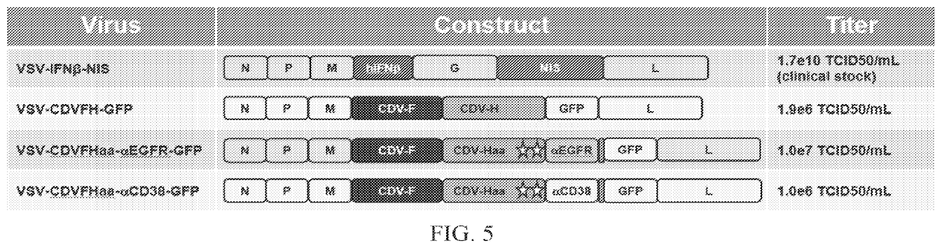

The following was performed to assess the use of Stealth 2.0 as an oncolytic agent. SCID mice bearing U266.B1 tumors were treated with a single intravenous dose of either MeV or Stealth 2.0 (FIG. 4A). The implanted tumors in the PBS-treated group continued to grow exponentially (FIG. 4B), and they all had to be sacrificed because of tumor burden by day 12 (FIG. 4C). On the contrary, both treatment groups had a slower tumor progression that resulted in a significantly increased median survival. Because similar oncolytic activity was observed for Stealth 2.0 and MeV, the latter being able to use in addition to CD46, the receptors Nectin-4 and SLAMF1, these results demonstrate that CD46 targeting is sufficient for tumor regression in a myeloma multiple model. Moreover, Stealth 2.0 demonstrated that it could substitute the current oncolytic MeV vaccine when high levels of neutralizing anti-measles virus antibodies are present in patients.

### CDV H/F Complexes can Retarget Other Mononegavirales

The following was performed to investigate the suitability of CDV H/F complexes to govern the tropism of VSVs, which are a member of the genus Lysasavirus, family Rhadoviridae. A VSV engineered to express interferon-beta (IFN-β) and the sodium iodide symporter (NIS), VSV-hIFNβ-NIS, (Naik et al. Mol. Cancer Ther., 17(1):316-326 (2018)) was obtained and modified by replacing the VSV-G polypeptide with CDV H and F polypeptides using the techniques described elsewhere (Ayala-Breton et al. Hum. Gene Ther., 23(5):484-91 (2012)). CDV F_{22458/16} polypeptide and either the parental CDV H₅₈₀₄ polypeptide (VSV-CDVFH-GFP) or the CDV H polypeptide retargeted against EGFR (VSV-CDVFHaal-αEGFR-GFP) or CD38 (VSV-CDVFHaal-αCD38-GFP) receptors were used (FIG. 5). In addition, each of the CDV H polypeptides contained a R529A mutation (CDV Haa) within the existing CDV H_{Y539A} background to abrogate interaction with canine SLAMF1.

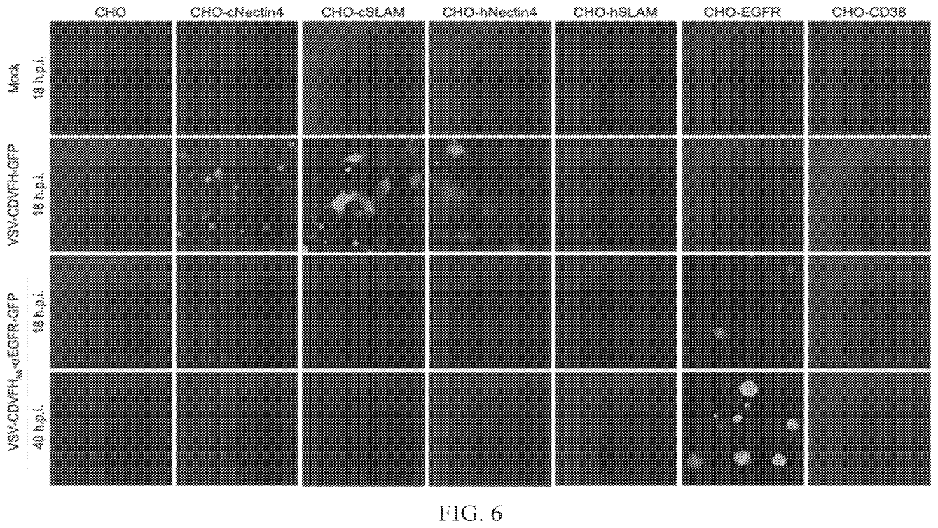

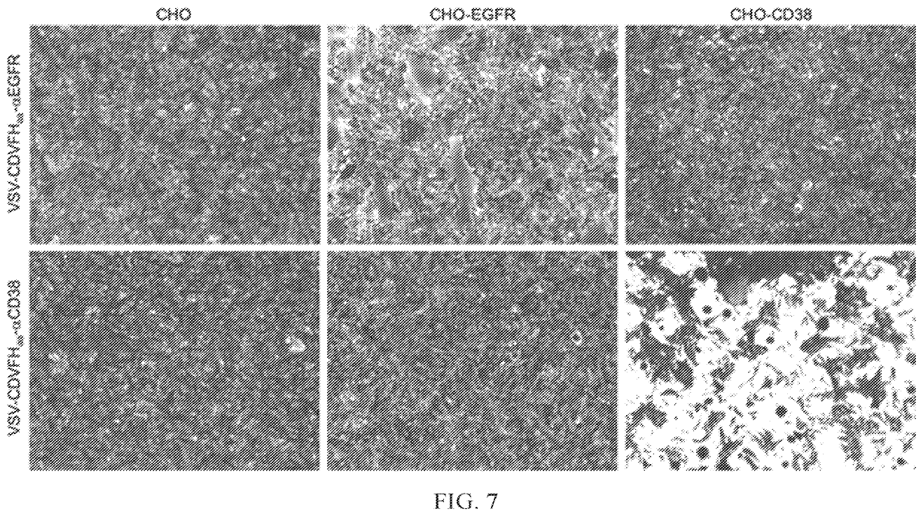

To confirm that the new envelope complex governed virus tropism, a panel of CHO cells expressing specific receptors were infected. As shown in FIG. 6, when the virus displayed the parental CDV F/H complex, GFP autofluorescence was observed in those cells expressing either Nectin-4 or canine SLAMF1 receptors. On the contrary, when the EGFR-specific scFv CDV H was present, only infection and GFP autofluorescence was observed in those cells expressing the EGFR receptor. Likewise, when the CD38-specific scFv CDV H was present, only infection and GFP autofluorescence was observed in those cells expressing the CD38 receptor. In this case, syncytia formation and cell-killing effect was observed in cells expressing CD38, but not with CHO cells expressing the EGFR receptors; whereas an opposite pattern was observed with the EGFR-specific virus (VSV-CDVFHaal-αEGFR-GFP) (FIG. 7). These results demonstrate in the context of rhabdoviruses that the CDV F/H complexes can be used to govern cell-entry and syncytia-formation in a receptor-specific manner via scFv display using a receptor of choice to re-direct virus tropism.

The use of this system as an oncolytic vector also was assessed in vivo (FIG. 8). Athymic nude mice bearing SKOV3ip.1 tumors were treated with a single dose of EGFR-targeted VSV or the VSV-hIFNβ-NIS currently undergoing testing in the clinic. Whereas VSV-hIFNβ-NIS did not improve survival versus PBS-treated controls, the EGFR-targeted VSV resulted in significantly improve survival (p<0.005). These results demonstrate that the targeted VSVs described herein can be used for oncology purposes.

### CD38 and EGFR Targeted MeV

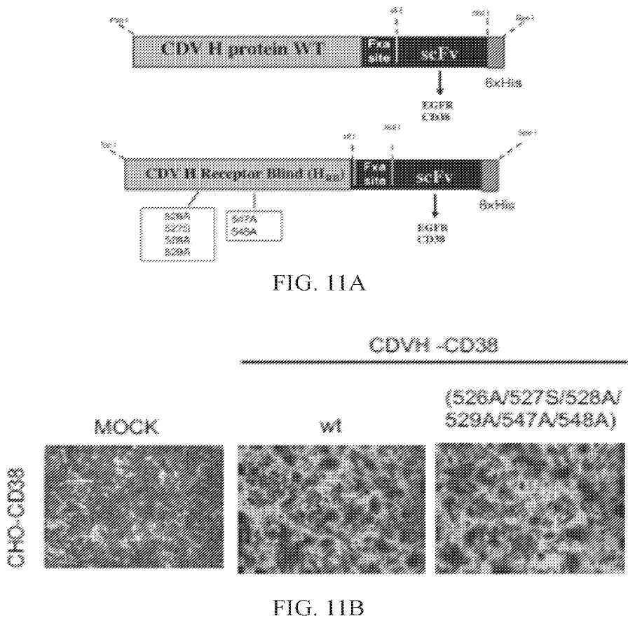

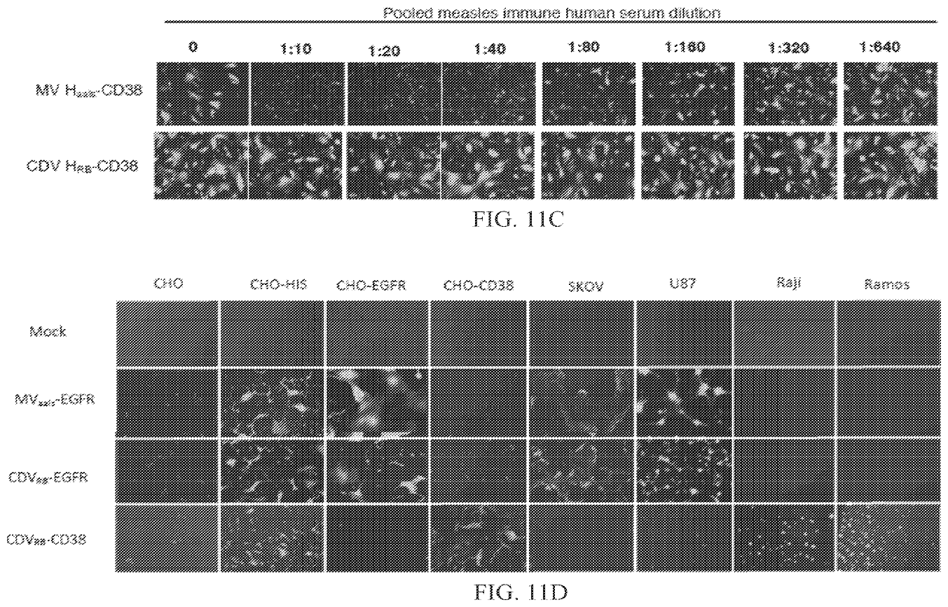

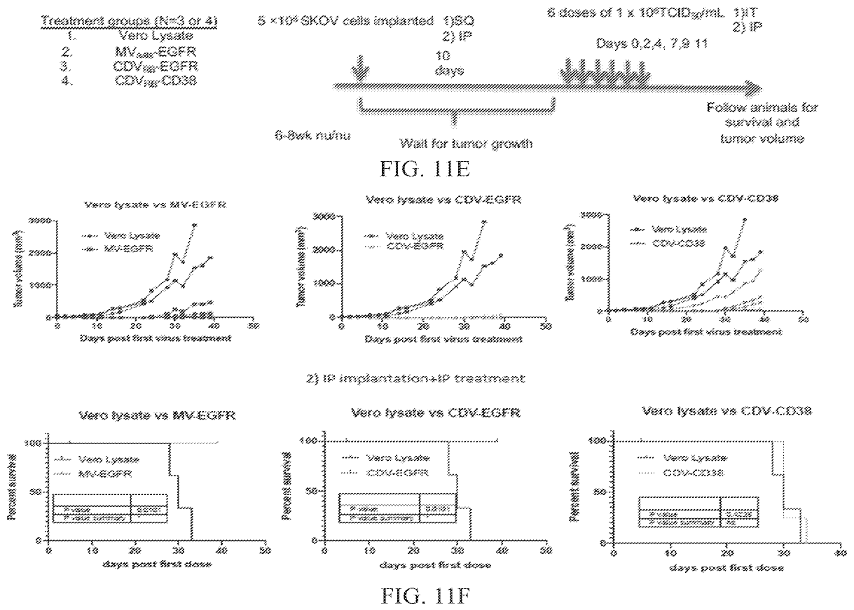

The following was performed to confirm that CD38 and EGFR targeting in the context of MeV can be achieved using CDV F and H polypeptides (FIG. 11). To increase safety, further mutations were inserted into the CDV H polypeptide to hamper a potential reversion to the use of natural receptors (Sawatsky et al. J. Virol., 92(15):e0069-18 (2018)). In addition to the R529A amino acid substitution, D526A, I527A, S528A, R529A, Y539A, Y547A, and T548 substitutions also were included (FIG. 11A). FIG. 11B shows that the introduction of these mutations in the context of CD38-targeted CDV H polypeptide did not influence fusogenicity capacity. Similarly, FIG. 11C demonstrates that the different point mutations did not influence the neutralization sensitivity of the polypeptides to measles immune human serum. Although the fusion activity of the CD38-targeted MeV H polypeptide was inhibited by pooled measles antisera up to 1:80 dilution, the homologous CD38-targeted CDV H polypeptide was not at the highest concentration tested of a 1:10 dilution. Similar to the case of Rhabdoviruses, Measles Virus incorporating the targeted CDV H/F complexes could infect and fuse cells in a CD38 or EGFR-specific manner (FIG. 11D). This infection-specificity also was observed with an array of tumor cell lines. Skov3pi and U87 cells (EGFR-positive) were infected by EGFR-targeted viruses, but not CD38-targeted viruses. On the contrary, Raji and Ramos cell lines (CD38 positive) were infected exclusively by the CD38-targeted viruses. As a control, cells expressing the 6×HIS pseudo receptor (“6×HIS” disclosed as SEQ ID NO. 21) were infected by all retargeted viruses. These results demonstrate that CDV F/H complexes can be used to avoid anti-measles immunity and drive cell-targeted entry and syncytia formation.

The in vivo oncolytic activity of CD38 and EGFR-targeted MeV were also investigated. SKOV3ip.1 tumors were implanted either subcutaneously or intraperitoneally into athymic nude mice, followed by virus treatment using the same route (FIG. 11E). Although CD38-targeted viruses showed some therapeutic potency, the potency exhibited by the EGFR-targeted viruses was superior (FIG. 11F). Conversely, CD38-targeted viruses exhibited no anti-tumor potency after intraperitoneal injection, whereas EGFR-targeted viruses resulted in complete tumor regression as observed by 100% percentage survival. Of note, no differences were observed between MeVs retargeted with either the MeV H/F complexes or CDV H/F. These results demonstrate that CDV H/F complexes can enhance the anti-tumoricidal properties of oncolytic measles virus without the problem of being neutralized by measles-induced neutralizing antibodies.

### Example 2—Further Analysis of CD46-Specific Oncolytic Measles Viruses Resistant to Neutralization by Measles-Immune Human Serum

This example repeats some of the information and results from Example 1 in addition to providing additional results.

### Heterologous Combinations of Wild-Type CDV Glycoproteins Result in Enhanced Cell Membrane Fusion

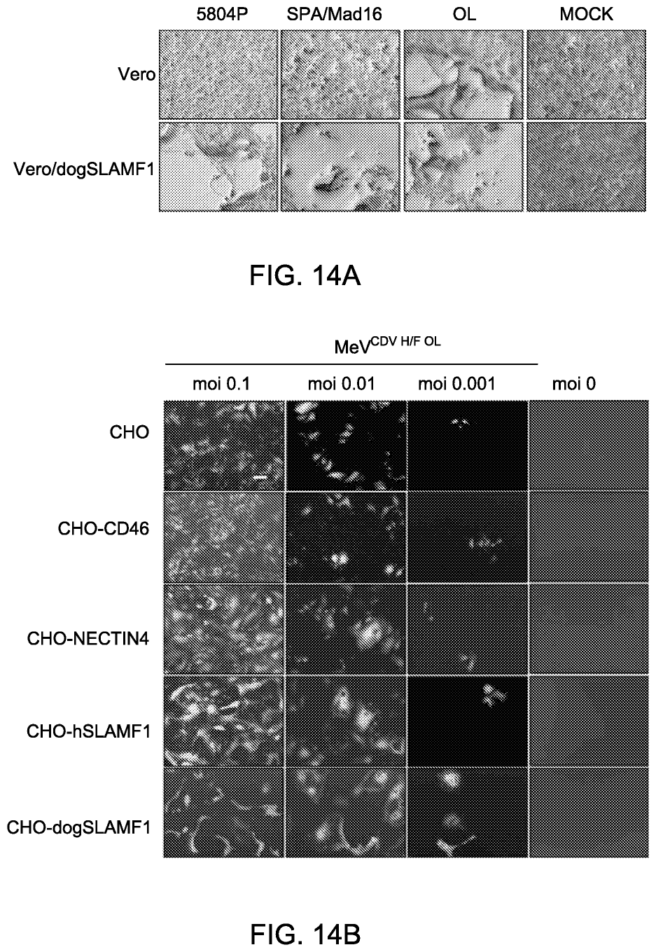

The MeV coat was replaced with an alternate viral coat that would enable the virus to evade neutralization by anti-measles antibodies. To do this, wild-type CDV was selected. While a strain of CDV approved for vaccine use exists (the Onderstepoort strain), this strain can use a currently unidentified receptor in addition to SLAMF1 and Nectin-4 (FIG. 14), which would have made it challenging to modify the viral tropism. Therefore, wild-type strains were focused on as they are known to interact exclusively with SLAM and nectin-4.

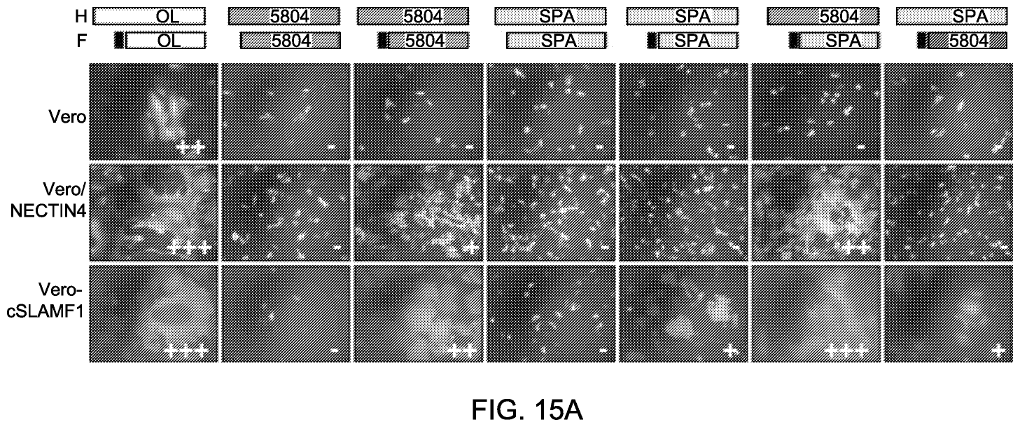

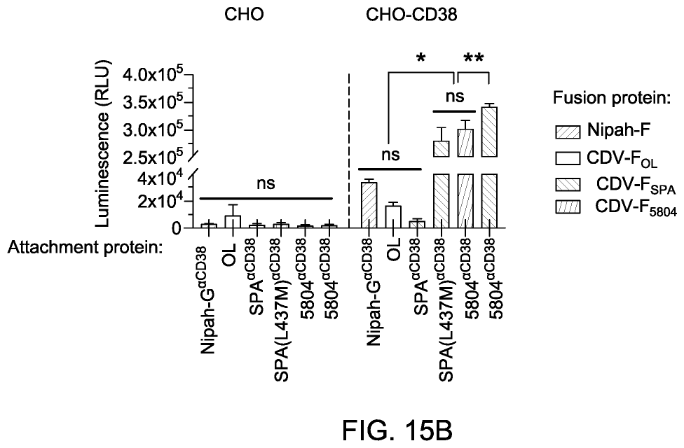

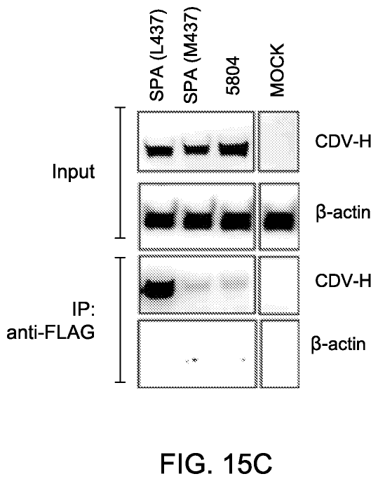

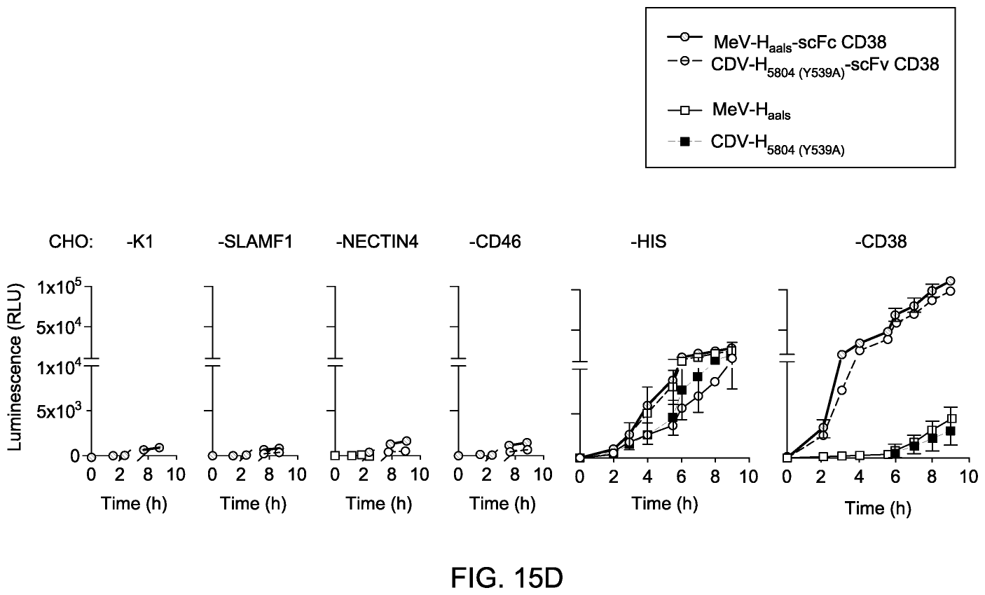

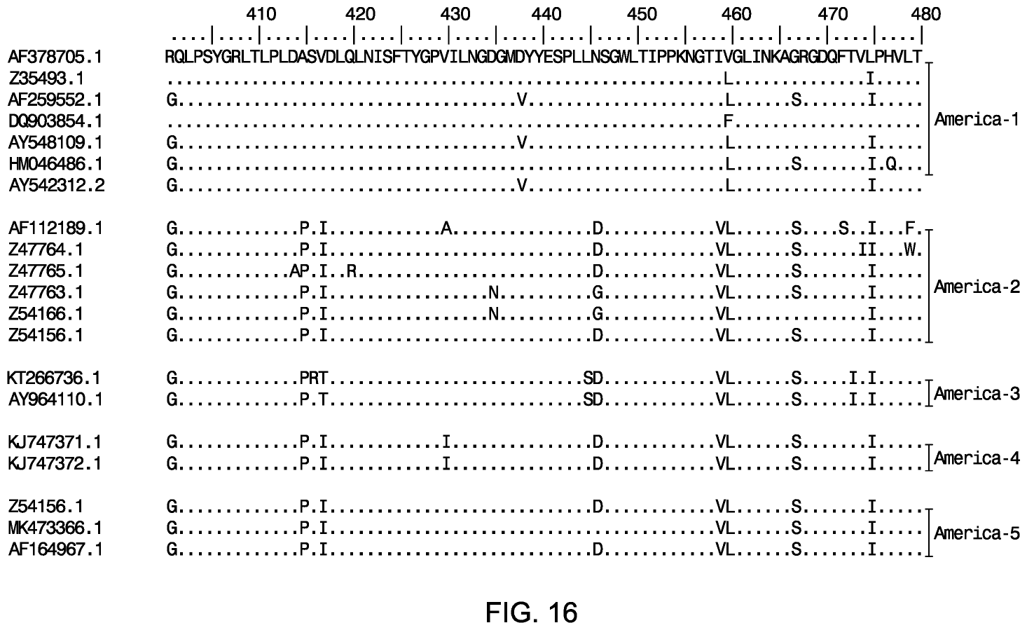

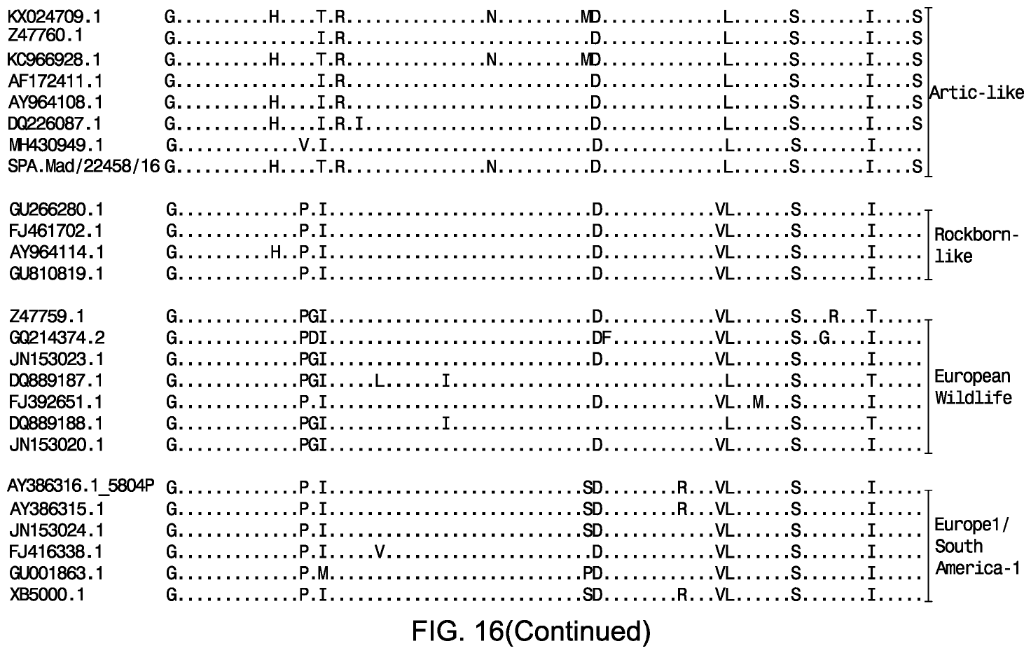

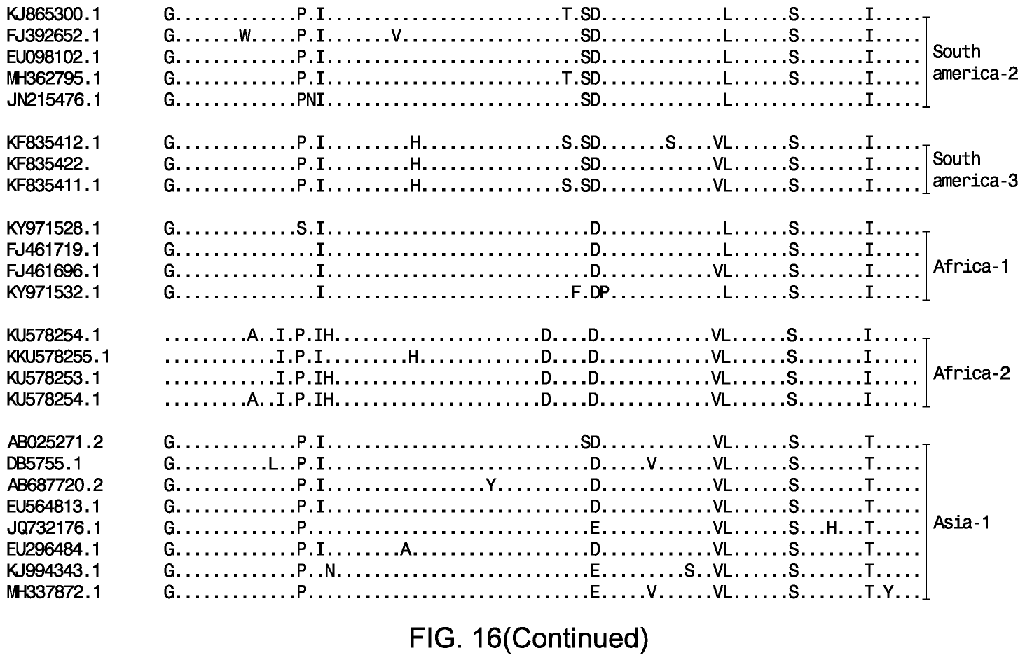

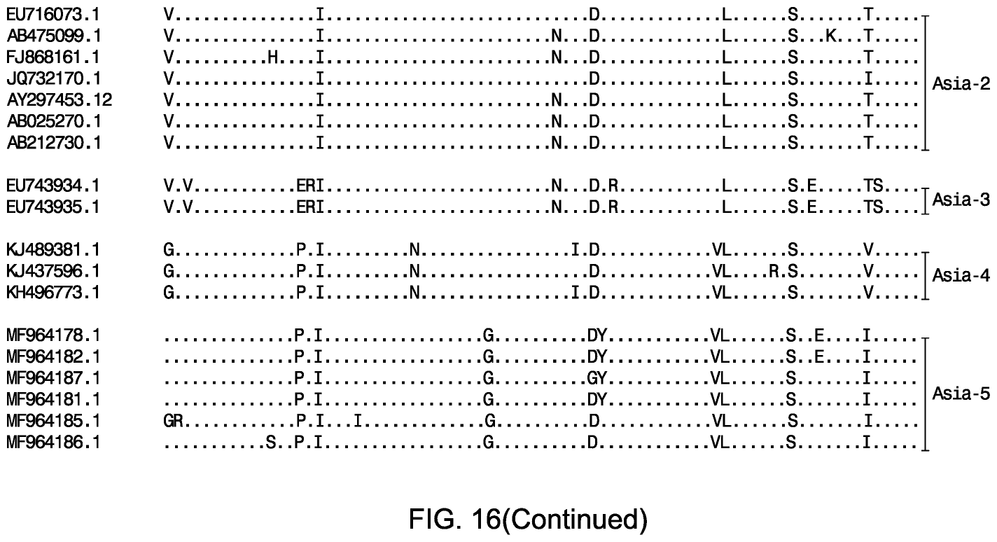

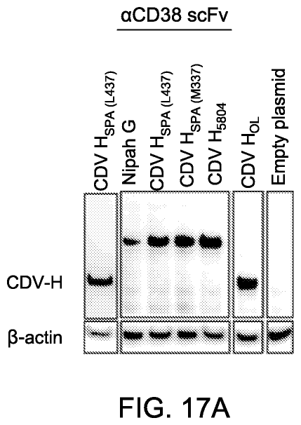

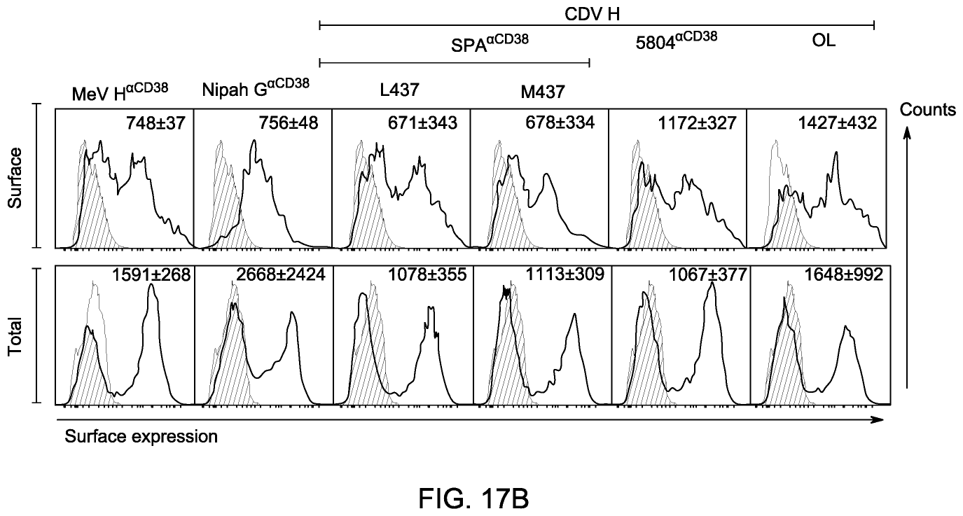

The following was performed to identify the most fusogenic CDV H/F glycoprotein pair. Different H/F combinations from the 5804P and SPA.Madrid/16 (hereafter named 5804 and SPA, respectively) isolates were transiently expressed in Vero cells expressing either SLAMF1 or NECTIN4 and assessed qualitatively the degree of cellular fusion (syncitia formation) induced by the viral proteins. Fusion activity for the CDV-H/F pairs was not observed when the 135 aa-signal peptide was maintained in CDV-F (FIG. 15A). When swapped by the homologous from MeV F, coexpression of H/F proteins from 5804P resulted in cell fusion in SLAMF1- and NECTIN4-expressing cells whereas coexpression of those from SPA promoted cell fusion exclusively in SLAMF1-expressing Vero cells. On the other hand, coexpression of the heterologous combination CDV-H 5804 and CDV-F SPA, but not CDV-H SPA and CDV-F 5804, resulted in syncytia formation in SLAMF1- and NECTIN4-expressing Vero cells. The data summarized above provided evidence in support of a fusion defect on CDV-H SPA. To begin to address whether the lack of fusion of CDV-H SPA on NECTIN4-expressing cells was due to a lower affinity for the receptor, fusion phenotypes were quantitatively compared via nonnatural receptors thus leveling the playing field for receptor binding affinity. This approach was to fuse a His-tagged CD38-specific scFv to the C-terminal domain of the receptor binding proteins and to determine fusion levels in CHO cells encoding either CD38 or the pseudoreceptor for the HIS-tag (CHO-αMIS). A L437M substitution was included into CDV-H SPA since L437 corresponded to a clone-specific mutation not present in any other CDV genetic group (FIG. 16). For comparison purposes, retargeted receptor binding proteins from other viruses were additionally included: MeV-H-and Nipah-G (Bender et al., PLoS Pathog., 12(6):e1005641 (2016); and Nakamura et al., Nat. Biotechnol., 22(3):331-6 (2004)). For added rigor to the comparisons, expression of the receptor binding proteins was initially analyzed by western blot and flow cytometric analysis, which demonstrated no significant impact on protein folding or surface expression (FIGS. 17A and 17B). When the CDV-H/F pairs from SPA proteins were expressed in CHO-C38, only the CDV-H SPA with M437L promoted fusion (FIG. 15B). Notably, no significant difference in fusion competence was observed between the two homotypic CDV-H/F pairs from the SPA or 5804P isolates. Surprisingly, the fusion activity triggered by the heterologous H/F combination CDV-H 5804/F SPA surpassed that achieved by the homologous combinations CDV-H/F 5804 and CDV-H/F SPA. For the retargeted Nipah G/F pair, although significant fusion levels were observed in CHO-CD38 cells (p<0.0001), they were insignificant when compared to those obtained by the unretargeted CDV H/F OL pair.

Based on this set of experiments, the hyperfusogenic CDV-H 5804/F SPA pair was selected for further investigation and modification.

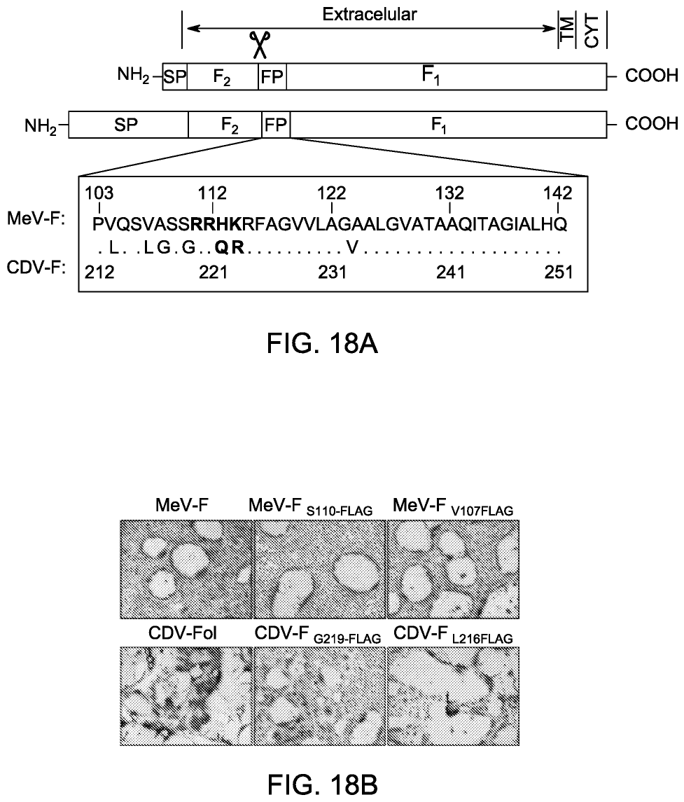

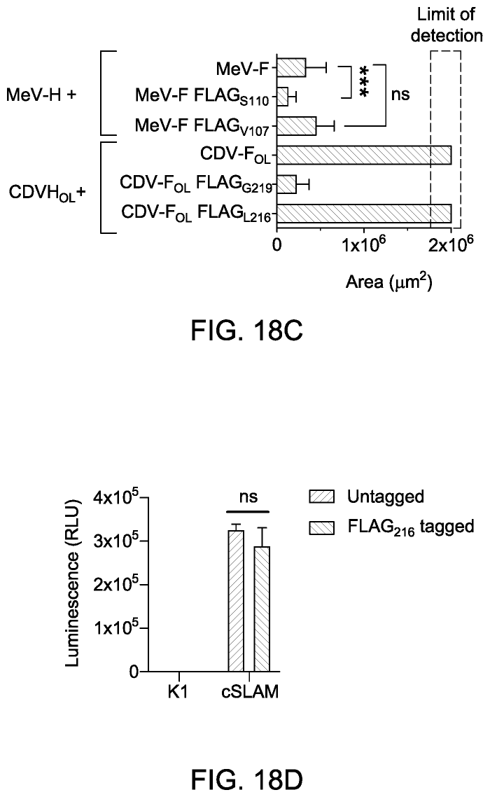

The Strength of the CDV H/F Interaction Inversely Correlates with the Cell-to-Cell Fusion Efficiency
The enhanced cell membrane fusion observed for the CDV-H 5804/F SPA pair might be related to a lower binding avidity at the H/F interface. This was based on the observation that H/F dissociation is essential for the fusion process (Plemper et al., J. Virol., 76(10):5051-61 (2002); and Bradel-Tretheway et al., J. Virol., 93(13) (2019)). In order to test the hypothesis, the relative strengths of association different combinations of CDV-H and F proteins were evaluated by coimmunoprecipitation (co-IP) assays. To facilitate detection, CDV-F SPA was fused to a FLAG-tag, which had no effect on the bioactivity of the protein (FIG. 18). The results, presented in FIG. 15C, showed that the presence of the M437L mutation in CDV-H SPA weakened its affinity for CDV-F SPA. In addition, the affinity of CDV-F SPA for CDV-H 5804 was slightly lower than that of CDV-F SPA for CDV-H SPA (FIG. 15C). Taken together, these data indicate that there is an inverse correlation between fusogenicity levels and the strength of the CDV-H/F interaction.

### Fully-Retargeted CDV Envelope Glycoproteins Display Comparable Fusion Activity to MeV Glycoproteins

The CDV-H proteins described above could still use NECTIN4 as a receptor (FIG. 15A). In order maximize retargeting efficiency, the CDV-H protein needed to be detargeted from this undesirable interaction with human cells. To determine whether the ablation of this natural tropism would affect the cell fusion induced by CDV-H/F binding to a nonnatural receptor, a Y539A mutation was introduced to CDV-H, which corresponds to Y543A in MeV-H, a mutation that was previously shown to abrogate NECTIN4-dependent fusion (Mateo et al., J. Virol., 87(16):9208-16 (2013)) while not affecting cell-surface expression (Sawatsky et al., J. Virol., 86(7):3658-66 (2012)). The fusion proficiency of CDV-H 5804 (Y539A) 5804/F SPA was then compared with that of a fully retargeted MeV-H/F pair (Nakamura et al., Nat. Biotechnol., 23(2):209-14 (2005)). For this, the quantitative and kinetic fusion assay based on a dual-split GFP/luciferase reporter protein was utilized. The data, presented in FIG. 15D, indicate that CD38-targeted CDV-H 5804 (Y539A) had no fusion activity in CHO-NECTIN-4 cells but did induce fusion in CHO-αHIS cells (construct CDV H 5804 (Y539A)/F SPA and CDV H 5804 (Y539A)αCD38/F SPA) and CHO-CD38 cells (CDV H 5804 (Y539A)αCD38/F SPA). Because the fusion activity of the NECTIN-4 blind CDV-H 5804/F SPA pair was comparable to that obtained with the fully retargeted MeV-H protein, it was concluded that CDV-H 5804 (Y539A) can efficiently retarget the CDV-H/F complex to specific receptors, and this protein was selected for incorporation in the fully retargeted virus.

### Binding Affinity Determines Efficient Retargeting of CDV H/F Complexes to CD46

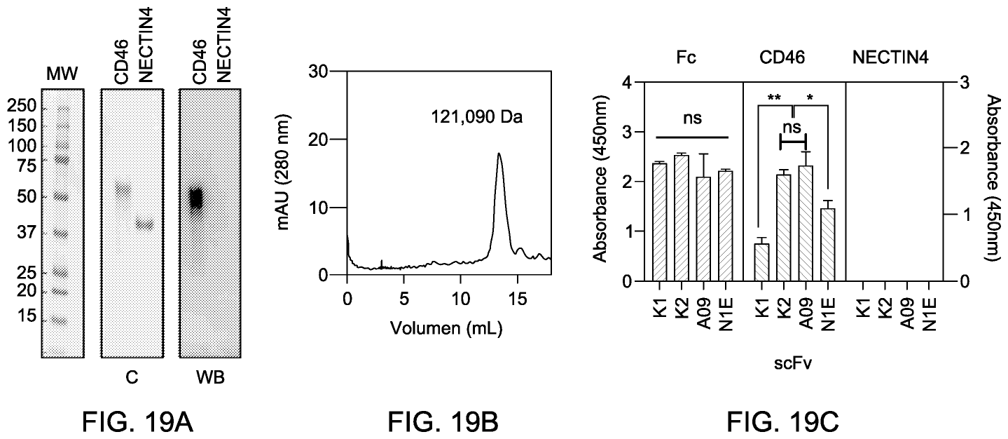

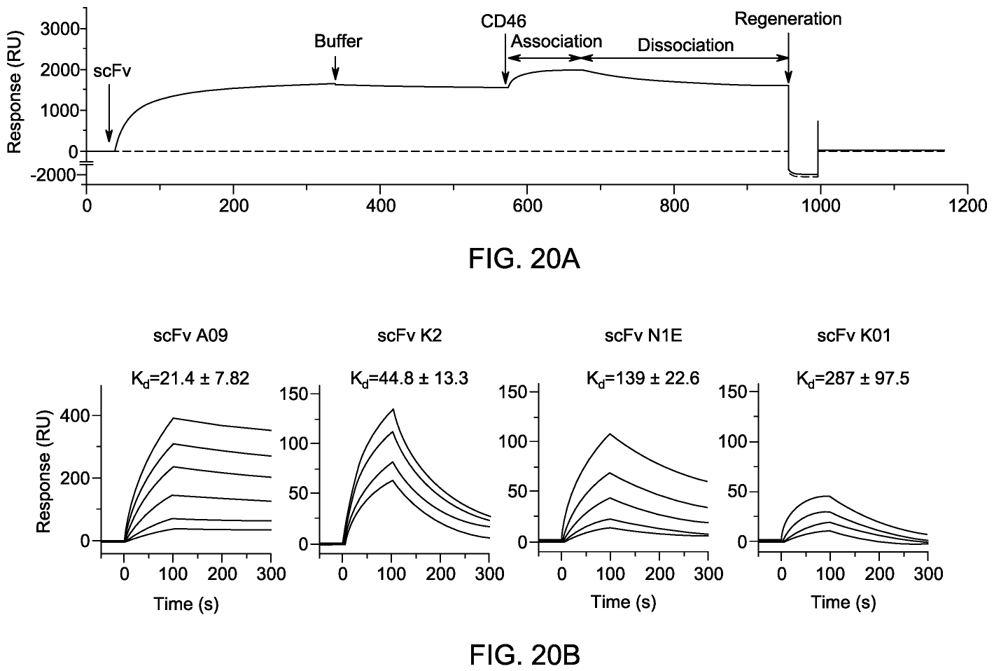

Given that the CDV-H protein selected as described above could be efficiently retargeted to CD38 by fusing a CD38-specific scFv, this protein was next retargeted to CD46 via display of a CD46-specific scFv. It was postulated that the C-terminal display on CDV-H of an scFv that recognized CD46 with sufficiently high binding affinity would result in CD46-mediated cell-to-cell fusion activity similar to that induced by the MeV H/F complex. To test this, an anti-CD46 scFv with high affinity for CD46 was identified by assessing binding to purified CD46 of several different scFv variants isolated from a phage antibody display library (FIG. 19). Surface plasmon resonance technology was applied using sensor chips with covalently immobilized anti-Fc antibody. Chimeric Fc-scFv fusion proteins were captured onto the sensor surface which was subsequently interrogated with soluble CD46 comprising SCR1-4 (FIG. 20A). Under these assay conditions, the results showed that the affinity constants (Kd) of the chimeric Fc-scFv fusion proteins displaying the A09 and K2 fragments were significantly stronger than those of the K01 and N1E fragments (A09>K2>N1E>K2), primarily due to increased association (A09) or lower dissociation rates (K2) (Table 4, FIG. 20B).

TABLE 4
Affinity and kinetic rate constants for single-chain variable
fragment binding to CD46.

| Interaction | Kₒₙ × 10⁴ (M⁻¹s⁻¹) | K_{off} × 10⁻³ (s⁻¹) | K_{D} (nM) |
|---|---|---|---|
| scFv A09-CD46 | 3.34 ± 1.68 | 0.606 ± 0.07 | 21.4 ± 7.82 |
| scFv K2-CD46 | 24.0 ± 3.79 | 10.8 ± 1.81 | 44.8 ± 13.3 |
| scFv N1E-CD46 | 3.69 ± 1.28 | 5.16 ± 2.21 | 139 ± 22.6 |
| scFv K1-CD46 | 4.13 ± 0.77 | 11.5 ± 2.58 | 287 ± 97.5 |

Association (Kₒₙ) and dissociation (K_{off}) reaction were determined with a BIACORE T100 instrument using a 1:1 Langmuir binding model.

The following was performed to determine whether fusion of a scFv to the CDV-H protein can support CD46-dependent fusion and if so, how CD46 binding affinity would affect cell fusion. The primary approach was to perform quantitative fusion assays for the detargeted CDV H [5804 (Y539)] and retargeted CDV-H [5804 (Y539)-scFv]/F SPA pairs and to compare them to the unmodified MeV/F complex on CHO cells and CHO cells expressing either NECTIN4 or CD46. All the proteins were expressed at comparable levels (FIG. 21). With the exception of scFv K01, all the other anti-CD46 scFvs allowed the CDV-H/F complex to induce cell-to-cell fusion in CHO-CD46 cells (FIG. 20C) and in a HeLa cell line with high CD46 expression (FIG. 21). Only the MeV-H/F complex induced cell-to-cell fusion in CHO-NECTIN-4 cells.

It was conclude from this set of experiments that there exists a binding affinity threshold for CD46-mediated cell-cell fusion through the retargeted CDV H/F complexes and above this threshold there is a positive correlation between binding affinity and intercellular fusion.

The CD46-Targeted CDV Envelope Glycoproteins are Efficiently Incorporated into MeV Virions and Mediate Virus Entry in Accordance with their Binding Affinity

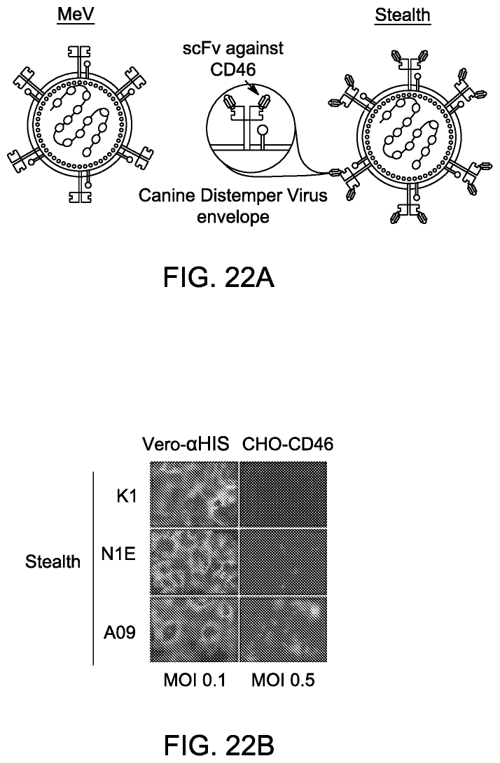

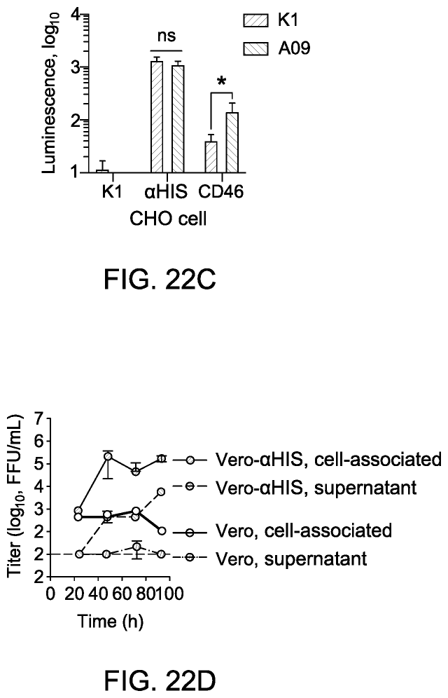

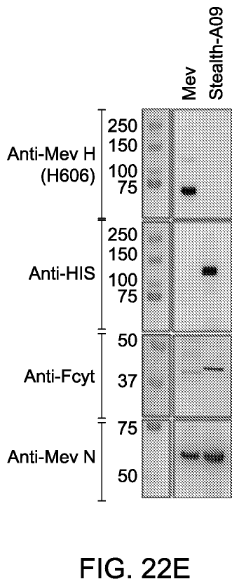

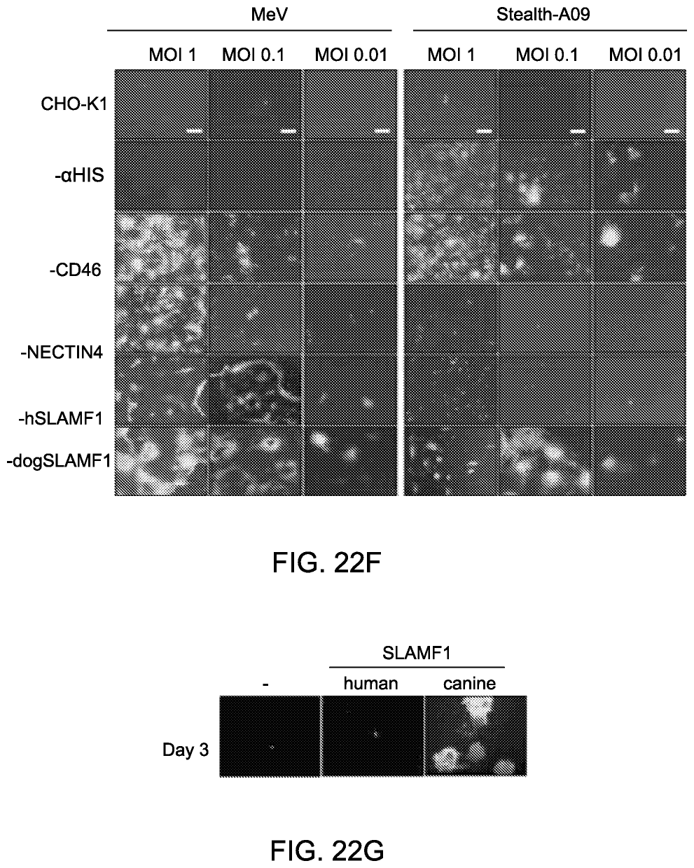

The following was performed to investigate whether higher receptor affinity translated into higher virus infectivity. To begin to address this question, a panel of isogenic MeVs were generated where the MeV coat was replaced with CDV-F SPA together with CDV-H 5804 (Y539A) displaying low (K1), intermediate (N1E) and high (A09) affinity scFv specific for CD46 (FIG. 22A). These “Stealth” viruses were further engineered to express either eGFP or firefly luciferase as reporter genes and were rescued on Vero-αHIS cells. The ability of the Stealth viruses to infect CHO-CD46 cells or the producer cell line (Vero-αHIS) were next assessed. The data, presented in FIG. 22B, showed that all three Stealth viruses efficiently induced syncytia formation on Vero-αHIS cells, but only Stealth-A09 formed syncytia on CHO-CD46 cells. Stealth-N1E gave rise to small clusters of unfused GFP-positive CHO-CD46 cells, whereas Stealth-K1 apparently failed to infect them (FIG. 22B). When using luciferase as a reporter for infection, CD46-dependent infection with Stealth-K1 was detected, but the luciferase levels were significantly lower than those obtained when cells were infected with Stealth-A09 (FIGS. 22C and 23), indicating that binding to CD46 determines the efficiency of virus entry.

Based on its superior CD46-dependent virus entry, Stealth-A09 (this virus was referred to as Stealth 2.0 in Example 1) was selected for further characterization. Stealth-A09 replicated in Vero-αHIS cells but not in the parental Vero cell line, indicating efficient virus replication through the HIS-pseudo receptor and a lack of interaction with CD46 from African green monkey (FIG. 22D). To estimate the relative particle-to-infectivity ratio of the Stealth-A09 in relation to the original MeV, western blot analysis was performed on virus preparations to detect the major structural protein (N), and no significant differences in expression levels were observed (FIG. 22E). Since equivalent numbers of virus particles were analyzed, the data suggest similar particle-to-plaque-forming unit (PFU) ratios for the two viruses, indicative of efficient incorporation into the virions of the foreign envelope. The receptor-specific tropism of the viruses was next fully examined by infecting a panel of CHO cells stably expressing αHIS, CD46, NECTIN-4, or either human or canine SLAMF1. The results, presented in FIG. 22F, showed that MeV infected CHO cells expressing the receptors CD46 and NECTIN-4 and either canine or human SLAMF1, whereas Stealth-A09 only infected CHO-αHIS, CHO-CD46, and CHO-dogSLAM cells, indicating that Stealth-A09 is efficiently retargeted against human CD46.

In keeping with a recent report showing that CDV-H does not bind human SLAMF1 (Fukuhara et al., Viruses, 11(8) (2019)), entry of MeV-Stealth-A09 into CHO-hSLAMF1 was not observed (FIG. 22F). However, adaptation to human SLAMF1 via a different CDV strain has been observed (Bieringer et al., PLoS One, 8(3):e57488 (2013)). Therefore, to assess the likelihood of MeV-Stealth adapting to human SLAMF1, the virus was passaged consecutively in Vero-hSLAMF1 cells, and viral tropism was analyzed. The infected cells were blind-passaged eight times at 5-day intervals before testing the tropism. This number of passages has been previously shown to suffice to induce cyclical adaptation of measles virus quasispecies (Donohue et al., PLoS Pathog., 15(2):e1007605 (2019)). As shown in FIG. 22G, at the end of this selective pressure, MeV-Stealth-A09 was still able to induce syncytia formation only in Vero-dogSLAMF1 cells, not in Vero or Vero-hSLAMF1 cells, where only discrete GPF-positive cells were observed. Thus, the data argue against a potential adaptation to allow use of the pathogenic human SLAMF1 receptor.

Collectively, these results indicate that the MeV H/F glycoproteins can be exchanged with the CD46-retargeted CDV-H/F glycoproteins and that cell entry is dependent on receptor affinity.

### MeV-Stealth Oncolytic Activity is Dependent on its CD46 Binding Affinity

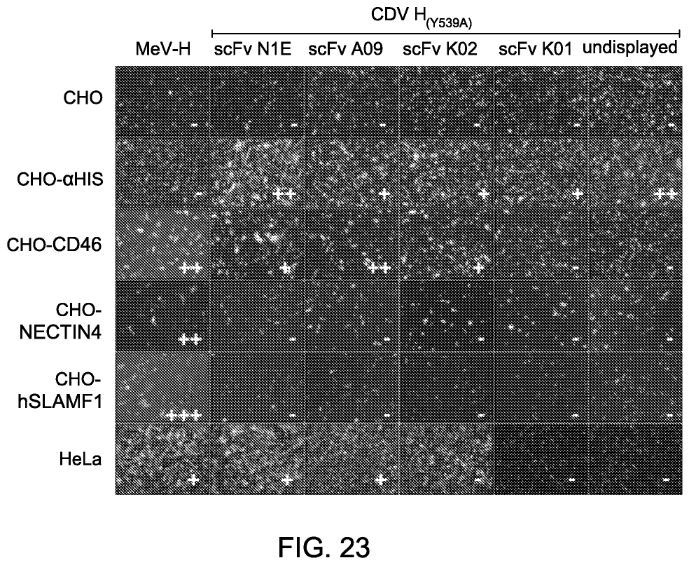

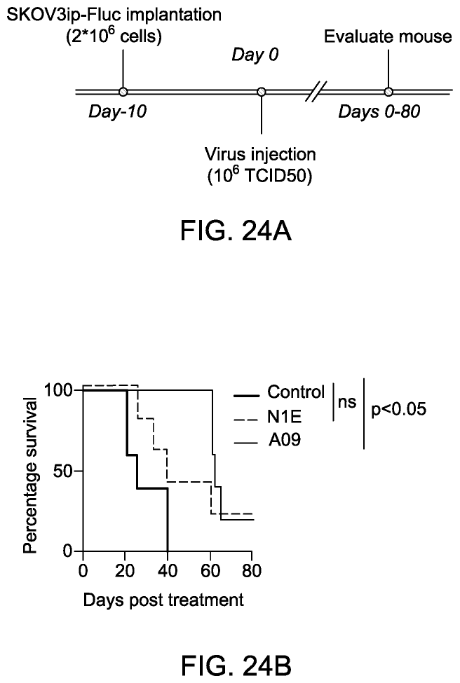

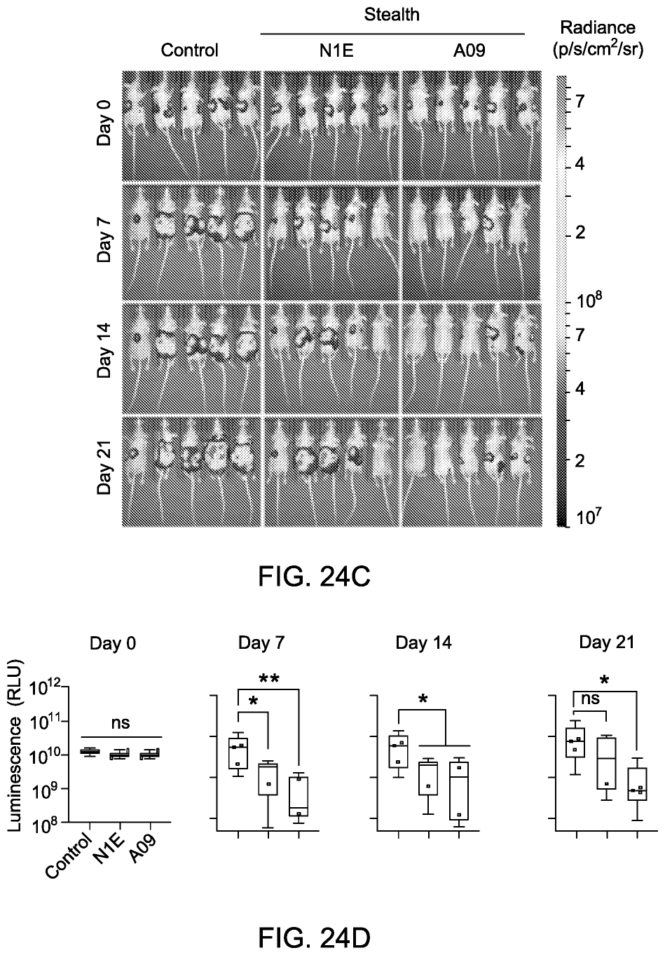

The following was performed to determine the antitumor potential of Stealth viruses and the role of CD46 binding affinity in vivo. For this, athymic mice bearing peritoneally disseminated SKOV3ip.1 tumors expressing the firefly luciferase gene (SKOV3ip.Fluc) were treated with a single intraperitoneal dose of saline or 10⁶ TCID₅₀ of Stealth-N1E and Stealth-A09 (n=5). Tumor burden was then monitored using in vivo bioluminescence imaging (FIG. 24A). By day 7, a comparable reduction in tumor burden was observed in animals that received either Stealth-N1E or Stealth-A09 when compared with the control group (FIG. 24B). However, the statistical significance in the reduction of tumor burden was lost for the Stealth-N1E group by day 21, whilst this was not the case for the Stealth-A09 group. Comparison of survival curves showed that only MeV Stealth-A09 increased mouse survival compared to the control group. Thus, Stealth-A09 shows oncolytic potency that appears dependent on its higher CD46 affinity.

### MeV-Stealth Achieves Oncolysis and Prolongs Survival of Myeloma and Ovarian Tumor-Bearing Mice

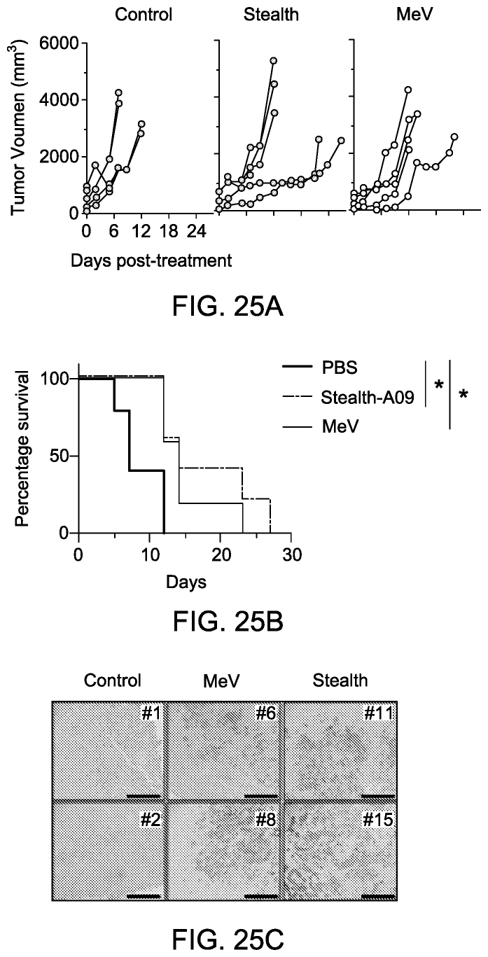

The following was performed to assess the utility of MeV-Stealth-A09 over MeV as an oncolytic agent. For this, we initially left untreated (PBS-treated group) or treated severe combined immunodeficiency (SCID) mice bearing subcutaneous human myeloma xenografts (derived from U266.B1 cells) with a suboptimal intravenous dose of MeV-Stealth or MeV. The tumors in the PBS-treated group continued to grow exponentially, and all mice had to be sacrificed because of the tumor burden by day 12 (FIG. 25A). Treatment with either MeV or MeV-Stealth-A09 slowed tumor progression, resulting in a significantly increased median survival time of 7 and 5 days, respectively (FIG. 25B). In order to assess whether the oncolytic effect was due to virus replication, histological analysis of explanted tumors was performed. The results, presented in FIG. 25C, strongly indicated that both viruses were able to home to tumor tissue. These results strongly suggested that CD46-targeted Stealth induces oncolysis at similar levels compared to MeV, which targets both CD46 and SLAMF1, thereby inducing tumor regression in this multiple myeloma model.

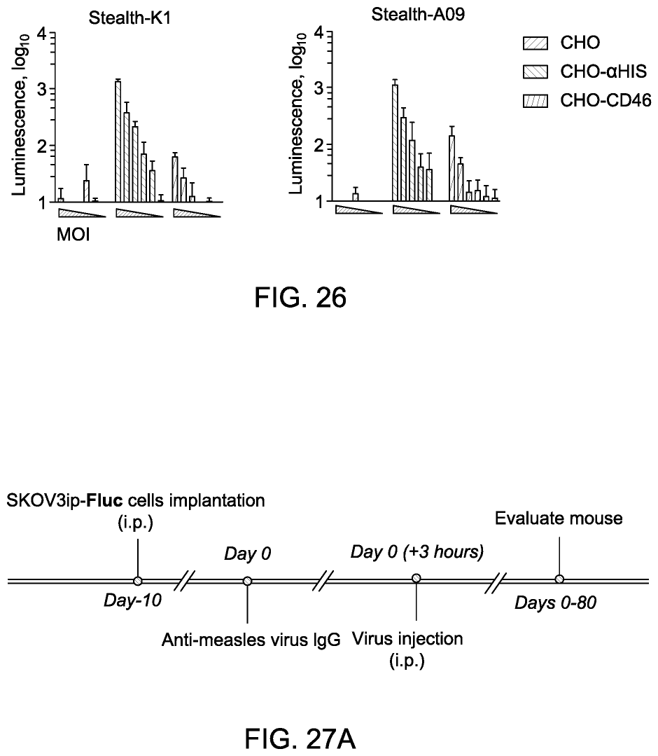

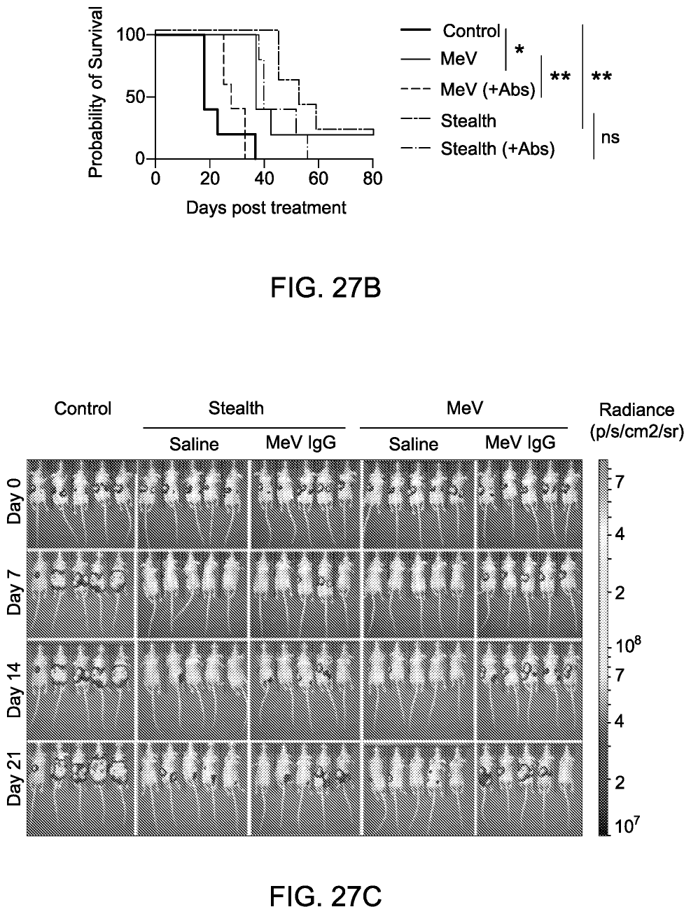

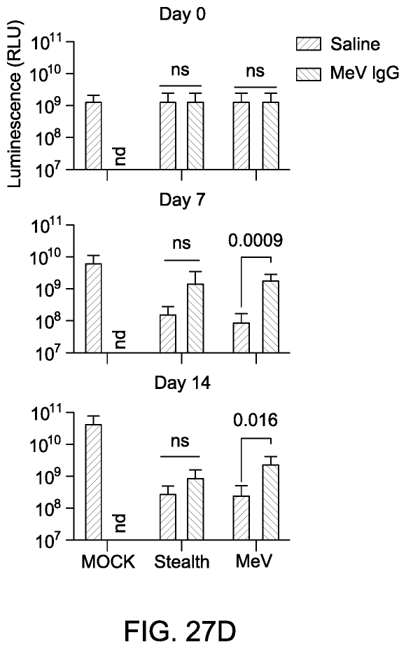

Next, the therapeutic effect of MeV-Stealth-A09 in prolonging survival in the presence of measles-immune serum was evaluated. Before embarking on this in vivo study, the neutralization sensitivity of recombinant viruses was first evaluated in vitro. The results, presented in FIG. 26, showed that MeV-Stealth-A09 was insensitive to the neutralizing activity of measles-immune human serum, whereas it was completely neutralized by CDV-immune ferret or mouse serum. The opposite pattern was observed essentially for MeV (FIG. 23). SKOV3ip.Fluc cells were then implanted into the peritoneal cavities of athymic nude mice, which received either PBS or measles-immune human serum prior to a single intraperitoneal injection of MeV or MeV-Stealth (FIG. 27A). Animals that did not receive any virus injection as well as those treated with MeV in the presence of immune serum exhibited high bioluminescence activity that continued to increase over time (FIG. 27B), indicating that MeV could not exert oncolysis in the presence of pre-existent immunity. On the contrary, Kaplan-Meier survival curves indicated that, in the absence of immune serum, both oncolytic virus (OV) treatments significantly prolonged mouse survival, with a median survival times of 37 days for the MeV-treated mice and 53 days for the Stealth-treated mice versus a median survival time of 18 days for the control mice (FIG. 27C). However, prolonged survival following MeV treatment was completely abrogated when mice received measles-immune serum, whereas MeV-Stealth treatment was still able to significantly prolong survival (median survival time of 28 days) with no statistical difference over treatment in the absence of immune serum.

These results demonstrate that CD46 targeting drives oncolyis in both a xenograft myeloma model and an orthotropic model of ovarian cancer, and that exchange of the MeV coat by the homologous CDV-H/F fusion apparatus shields MeV from MeV-immune human serum.

### Methods and Materials

### Cell Lines

Baby hamster kidney cells (BHK, Cat. #CCL-10, ATCC, Manassas, VA, USA), Human kidney epithelial cells (HEK293T) obtained from Dr. Franųcois-Loïc Cosset (Universitd de Lyon), and the human ovarian cancer cell line SKOV3ip.1-Fluc (Mader et al., Clin. Cancer Res., 15(23):7246-55 (2009)) were maintained in Dulbecco's modified Eagle's medium (DMEM; Cat. #SH30022.01, GE Healthcare Life, Pittsburgh, PA, USA) supplemented with 5% fetal bovine serum (FBS; Cat. #10437-028; Thermo Fisher Scientific, Waltham, MA, USA). Vero African green monkey kidney cells (Vero, ATCC, Cat. #CCL-81) and their derivatives (expressing human NECTIN-4 (Noyce et al., Virology, 436(1):210-20 (2013)), human SLAMF1 (Ono et al., J. Virol., 75(9):4399-401 (2001)) or a membrane-anchored single-chain variable fragment (scFv) specific for a hexahistidine peptide (6×HIS-tag (SEQ ID NO: 21)) (Nakamura et al., Nat. Biotechnol., 23(2):209-14 (2005)) were cultured in DMEM (Cat. #SH30022.01, GE Healthcare Life Sciences) as described elsewhere (Munoz-Alia et al., Viruses, 11(8) (2019)). Vero cells constitutively expressing the canine SLAMF1 molecule (Vero-dogSLAMF1) were generated by transduction and puromycin selection of a second-generation lentiviral vector (kindly provided by Dr. Lukkana Suksanpaisan [Imanis Life Science, Rochester, MN, USA]) encoding, under the control of the spleen focus-forming virus promoter, a codon-optimized SLAMF1 molecule from Canis lupus familiaris (GenBank NP_001003084.1) with an N-terminal FLAG-tag sequence (DYKDDDD (SEQ ID NO: 28)). Cells were maintained in DMEM supplemented with 5% FBS. The Chinese hamster ovary (CHO) cell line, CHO-CD46 cells, CHO-hSLAMF1 cells, CHO-dogSLAMF1 cells, CHO-NECTIN4 cells, CHO-αHIS cells, CHO-CD38 cells and the human myeloma cell line U266.B1 (kindly provided to us by Dr. David Dingli [Mayo Clinic, Rochester, MN]) were grown in RPMI 1640 medium supplemented with 10% FBS. Cells were incubated at 37° C. in 5% CO2 with saturating humidity.

### Plasmids and Construction of Full-Genome Recombinant Measles Virus (MeVP)

To generate canine distemper virus (CDV) SPA.Madrid/22458/16 expression plasmids, total RNA was extracted from CDV SPA.Madrid/22458/16 isolate-infected Vero/dog SLAMF1 cells (passage 1) using the RNeasy Mini Kit (Qiagen, Hilden, Germany). Both the CDV-hemagglutinin (H) and CDV-fusion (F) genes were reverse transcribed with SuperScript III Reverse Transcriptase (Cat. #11752050, Thermo Fisher Scientific) and amplified by PCR with the following primers: CDVH7050(+): 5′-AGAAAACTTAGGGCTCAGGTAGTCC;-3′ (SEQ ID NO: 22) CDVH8949(−): 5′-TCGTCTGTAAGGGATTTCTCACC-3′ (SEQ ID NO: 23); CDVF4857(+): 5′-AGGACATAGCAAGCCAACAGG-3′ (SEQ ID NO: 24) and CDVH7050(−): 5′-GGACTACCTGAGCCCTAAGTTTCT-3′ (SEQ ID NO: 25). PCR products were sequenced directly by Sanger sequencing (Genewiz, Plainfield NJ, USA) and cloned into the pJET1.2 vector (Thermo Fisher Scientific). Next, the CDV-H open reading frame was PCR amplified with a forward primer (5′-CCG GTA GTT AAT TAA AAC TTA GGG TGC AAG ATC ATC GAT AAT GCT CTC CTA CCA AGA TAA GGT G-3′ (SEQ ID NO: 26)) and a reverse primer (5′-CTA TTT CAC ACT AGT GGG TAT GCC TGA TGT CTG GGT GAC ATC ATG TGA TTG GTT CAC TAG CAG CCT CAA GGT TTT GAA CGG TTA CAG GAG-3′ (SEQ ID NO: 27)) and cloned into a PacI and SpeI-restricted (New England Biolabs, Ipswich, MA, USA) pCG vector (Cathomen et al., J. Virol., 72(2):1224-34 (1998)) using an InFusion HD kit (Takara, Shinagawa, Tokyo, Japan). The primers contained the PacI and SpeI restriction sites (underlined) as well as the coding sequence for the untranslated region of MeV-H (italics). Similarly, the CDV-F open reading frame (amino acid residues 136-662) was cloned into the HpaI/SpeI-restricted pCG-CDV-F plasmid (von Messling et al., J. Virol., 75(14):6418-27 (2001)). The resulting plasmid pCG-CDV-F SPA.Madrid/22458/16 contained coding sequences for the MeV-F untranslated region and signal peptide.

Expression plasmids for the CDV-H/F Onderstepoort vaccine and 5804P isolate (von Messling et al., J. Virol., 75(14):6418-27 (2001)), as well as MeV Nse strain, were described elsewhere (Cathomen et al., J. Virol., 72(2):1224-34 (1998)). The signal peptide for CDV-F 5804 was replaced with heterologous MeV-F as described above for CDV-F SPA.Madrid/22458/16. The open reading frames of the Nipah-G and Nipah-F glycoprotein genes were amplified from purchased RNA templates (Cat. #NR-37391, BEI Resources), and the Nipah-F gene (GenBank AF212302.2) was inserted into the pCG vector using the NarI and PacI sites. Retargeted versions of the H/G proteins were generated by inserting the homologous PacI/SfiI-digested PCR product into pCGHX α-CD38 (Peng et al., Blood, 101(7):2557-62 (2003)). Insertion of the coding sequence for an scFv recognizing CD46 was performed by exchanging the anti-CD38 scFv via the SfiI and NotI restriction sites. Site-directed mutagenesis (QuickChange Site-Directed Mutagenesis Kit, Agilent Technologies, Santa Clara CA, USA) was used to ablate the tropism of H and remove the SpeI site in CDV-F.

The viruses used in this example were derived from the molecular cDNA clone of the Moraten/Schwart vaccine strain pB(+)MVvac2(ATU)P, with an additional transcriptional unit downstream of the phosphoprotein gene (Cathomen et al., J. Virol., 72(2):1224-34 (1998); and Munoz-Alia et al., Viruses, 11(8) (2019)). To avoid plasmid instability upon propagation in bacteria and increase virus rescue efficiency, the plasmid backbone was replaced with the pSMART LCkan vector (Cat. #40821-1; Lucigen, Middleton, WI, USA), with an optimized T7 promoter followed by a self-cleaving hammerhead ribozyme (Hrbz) (Beaty et al., mSphere, 2(2) (2017); and Munoz-Alia et al., Viruses, 11(8) (2019)). eGFP or firefly luciferase were cloned into the infectious clone by using the unique MluI/AatII restriction sites. Rescue of rMeVs was carried out employing the START system (Nakamura et al., Nat. Biotechnol., 23(2):209-14 (2005)).

### Expression of Recombinant Proteins

A plasmid encoding the CD46-Fc fusion protein was produced by fusing the CD46 ectodomain (residues 35-328) with the Fc domain of IgG1 (pfc1-hg1e3; InvivoGen, San Diego, CA, USA). The scFvs K1, K2, and A09 were designed with the VL and VH sequences separated by a GSSGGSSSG flexible linker (SEQ ID NO: 29), codon-optimized, synthesized and cloned into pUC57-Kan (GenScript). A fourth scFv (NIE) was designed with the VH and VL sequences separated by an SSGGGGS linker (SEQ ID NO: 30), codon-optimized, synthesized by Creative Biolabs (Shirley, NY) and cloned into pCDNA3.1+(Invitrogen). For the IgG constructs, scFvs were cloned into the unique AgeI and KpnI sites of pHL-FcHIS (Cat. #99846, Addgene, Cambridge, MA, USA), harboring the coding sequence for a secretion signal and a C-terminal human Fc region followed by a 6×HIS-tag (SEQ ID NO: 21). The recombinant proteins were expressed by transfecting Expi293F suspension cells (Thermo Fisher) in serum-free Expi293 expression medium (Thermo Fisher) in shaker flasks following the manufacturer's instructions. The culture supematants containing the recombinant proteins were collected and passed through a Protein G chromatography cartridge (Cat.#89926, ThermoFisher). Bound recombinant proteins were eluted with 0.1 M glycine (pH 2.0), followed by immediate neutralization with 1 M Tris (pH 8.0), and the isolated proteins were concentrated with an Amicon Ultra centrifugal concentrator (Millipore Sigma, Burlington, MA, USA). CD46 and NECTIN4 were released from the Fc region by incubation with HRV 3C Protease (Thermo Fisher) at a 1:200 ratio. A final purification step was performed using a Superdex 75 10/300 gel filtration column (GE Healthcare) equilibrated in phosphate-buffered saline (PBS). Protein concentrations were calculated from the protein extinction coefficient as determined from the amino acid composition.

### Fusion Assays

Cells were transfected using Fugene HD (PROMEGA, Fitchburg WI, USA) or TransIT-LT1 transfection reagent (Mirus Bio LLC, Madison WI, USA). For a quantitative fusion assay, a dual-split reporter system (Kondo et al., J. Biol. Chem., 285(19):14681-8 (2010); and Ishikawa et al., Protein Eng. Des. Sel., 25(12):813-20 (2012)) was used as described elsewhere (Munoz-Alia et al., Viruses, 11(8) (2019)), using BHK cells as the effector cells. For semiquantitative assessment of fusion, Vero cells and derivative cell lines were transfected with a total of 0.1 μg of DNA (1:1 ratio of H and F expression plasmids), including a GFP expression plasmid for added visualization of syncitia formation. Images were obtained with a microscope (Eclipse Ti-S; Nikon) at 40×or 100× magnification.

### Expression Analysis of Morbillivirus Attachment Proteins

For assessment of the level of the H polypeptide, transfected cells were analyzed by flow cytometry or cellular enzyme-linked immunosorbent assay (CELISA) using an anti-6×HIS-tag monoclonal antibody (Cat. #130-120-787, Miltenyi Biotec or Cat. #MA1-135, Thermo Fisher Scientific), as described elsewhere (Munoz-Alia et al., Viruses, 11(8) (2019); and Saw et al., Methods, 90:68-75 (2015)). For analysis of total protein expression by flow cytometry (FACSCanbt, BD Biosciences, San Jose, CA, USA), cells were treated with the eBioscience Intracellular Fixation & Permeabilization buffer (Cat. #88-8823-88, Thermo Fisher Scientific).

### Envelope Glycoprotein Coimmunoprecipitation (Co-IP)

Three micrograms (1 μg of H and 2 μg of F) of total DNA were transfected into HEK293T cells (4e5 cells). After 24 hours, the cells were washed twice with PBS and treated with the cross-linker 3-3′-diothiobis (sulfosuccinimidyl propionate) (DTSSP; Cat. #21578, Thermo Fisher Scientific) at 1 mM, followed by quenching with 20 mM Tris/HCl (pH 7.4) and lysis with 0.4 mL of M-PER mammalian protein extraction reagent (Thermo Fisher Scientific) containing a 1× Halt protease and phosphatase inhibitor cocktail (Cat. #1861281, Thermo Fisher Scientific). Soluble fractions were collected after centrifugation at 10,000×g for 10 minutes at 4° C., and one-thirtieth of the volume was set aside as the cell lysate input. The rest was incubated with 0.5 μg of anti-FLAG monoclonal antibody M2 (Sigma-Aldrich) and EZview red protein G affmity gel (Sigma-Aldrich, St. Louis, MO, USA). The precipitated material was washed (20 mM Tris-HCl, pH 7.4, 140 mM sodium chloride) and denatured by boiling in laemmli buffer containing β-mercaptoethanol.

### SDS-PAGE and Immunoblotting

Samples were fractioned by gel electrophoresis on a 4 to 12% NuPAGE Bis-tris gel (Thermo Fisher) and transferred to polyvinylidene difluoride (PVDF) membranes using an iBLOT 2 dry blotting system (Cat. #IB21001, Thermo Fisher Scientific). The protein material was detected though incubation with the antibodies anti-MeV-H606 (Hudacek et al., Cancer Gene Therapy, 20(2):109-16 (2013)), anti-MeV-F431 (von Messling et al., J. Virol., 78(15):7894-903 (2004)), anti-Fcyt (von Messling et al., J. Virol., 75(14):6418-27 (2001)), anti-MeV-N(Toth et al., J. Virol., 83(2):961-8 (2009)), anti-HIS (Cat. #A01857-40, GenScript, Piscataway NJ, USA), anti-β-actin (Cat. #A3854, Sigma-Aldrich), and anti-CD46 (Cat. #sc-7056, Santa Cruz, Dallas TX, USA). Immunoblots were visualized using a rabbit horseradish peroxidase (HRP)-conjugated secondary antibody and KwikQuant Imager (Kindle Bioscience LLC, Greenwich CT, USA). Representative results of two independent repeats are shown. Band quantification was carried out using the KwikQuant Image Analyzer 1.4. (Cat. #D1016, Kindle Biosciences, LLC)

### Antibody Binding Assay

An enzyme-linked immunosorbent assay (ELISA) was used to measure the binding of scFvs to CD46. Nunc-Immuno MicroWell 96-well solid plates were coated overnight at 4° C. with 1 μg of purified CD46 or N4 in 0.05 M carbonate-bicarbonate buffer, pH 9.6. (Cat. #E107, Bethyl Laboratories, Montgomery TX, USA). Purified scFv-Fc fusion proteins were then diluted in PBS and added at a concentration of 12.5 μg/mL. Bound antibodies were detected with a secondary anti-human IgG (Fc-specific) HRP-conjugated antibody (1:70,000; Cat. #A0170, Sigma-Aldrich). In parallel, 125 ng of scFv-Fc fusion protein was first bound to wells, and protein levels were monitored by measuring the optical density (OD490 nm) after incubation with the secondary antibody alone.

### Surface Plasmon Resonance (SPR)

The interaction between the scFv A09, 2B10, K1, K2, and CD46 was measured using series S CM5 sensor chips on a Biacore T-100 system (GE Healthcare, Waukesha, WI, USA). For A09, N1E, K1, and K2, 50 μg/mL of an anti-FC antibody (MAB1302, EMD Millipore, Burlington, MA, USA) diluted into 10 mM NaAcetate, pH 4.5, were immobilized to the active and the reference channel of the CM5 chip using amine coupling kit reagents (EDC (1-ethyl-3-[3-dimethylaminopropyl]carbodiimide), NHS (N-hydroxysuccinimide) and ethanolamine). The immobilization of the antibody resulted in ˜12000 response units. The interactions between CD46 and the anti-FC antibody captured scFv were measured at 25° C. with a data rate of 10 Hz using HBS EP buffer (0.01 M HEPES pH 7.4, 0.15 M NaCl, 3 mM EDTA, 0.005% v/v Surfactant P20). Each binding cycle began with the loading of 15 μg/mL of the scFv onto the active channel for 300 s at a flow rate of 10 μL/min. After 100 s of buffer wash and a 120 s stabilization period, CD46 (concentration range 50 nM-1000 nM for A09, 37.5 nM-100 nM for K2 and 50-1000 nM for N1E and K1) was flown over the active and the reference channel for 100 s at a flow rate of 40 μL/min. The association phase was followed by a 200 s dissociation period followed by a 60 s injection of 10 mM Glycine pH 2.0 at a flow rate of 30 μL/min, to regenerate the surface immobilized anti-FC antibody. All sensograms were fitted with a 1:1 binding model using the Biacore T100 evaluation software v2.04.

### Infections and Virus Growth Kinetic Analysis

For virus infections, cells were infected at the indicated MOIs for 90 min at 37° C. in Opti-MEM I reduced-serum medium. After the absorption phase, we removed the inoculum, washed, and added viral growth media (DMEM+5% FBS). When using eGFP-expressing viruses, fluorescence microscopy photographs were taken 48 hours post-infection. For infections with Fluc-expressing viruses, luciferase expression was measured using an Infinite M200 Pro multimode microplate reader (Tecan Trading AG) after adding 0.5 mM of D-Luciferin to the infected cells.

For virus growth kinetic analysis, Vero cells and derivative cell lines seeded in 6-well plates 16-18 hours prior to infection were infected at a multiplicity of infection (MOI) of 0.03 for 90 min in Opti-MEM (Cat. #31985070, Thermo Fisher Scientific). The inoculum was then removed, and the cell monolayers were washed three times with Dulbecco's phosphate-buffered saline (DPBS; Cat. #MT-21-031-CVRF, Mediatech, Inc., Manassas, VA, USA), and the medium was replaced with 1 mL of DMEM supplemented with 5% FBS. At the indicated time points, cell supernatants were collected, and cells were scraped into 1 mL of Opti-MEM, followed by 3 freeze/thaw cycles. Cell debris was removed by centrifugation (2,000×g for 5 min), and virus titers were determined in Vero-αHIS cells.

### Immunization Studies

Four to six-week-old male and female HuCD46Ge-IFNARKO mice (Mrkic et al., J. Virol., 74(3):1364-72 (2000)), deficient for type I IFN receptor and transgenically expressing human CD46, were inoculated intraperitoneally (i.p.) with 1×10⁵ TCID₅₀ particles of MeV or Stealth-A09. On day 28, serum samples were collected and stored at −20° C. until assessed for neutralizing antibodies.

### Neutralization Assays

A fluorescence-based plaque reduction microneutralization (PRMN) assay was carried out as described elsewhere (Munoz-Alia et al., J. Virol., 91(11) (2017)). Briefly, Vero-αHIS cells were seeded in a 96-well plate, and serial dilutions of serum samples were premixed for 1 hour at 37° C. with virus inoculum before they were added to the cells. The data were plotted as the log(dilution of serum) vs. the normalized response (variable slope) present with GraphPad software (Prism 8), and the neutralization dose 50% was calculated (ND50). Inclusion of the 3rd World Health Organization International serum standard (3IU/mL) enabled conversion of antibody titers to mIU/mL by calculation of the unitage constant (Haralambieva et al., Vaccine, 29(27):4485-91 (2011)). Pooled human serum from 60-80 donors that had blood type AB (Cat. #HS1017; Lot #C80553, Valley Biomedical Inc., Winchester, VA, USA) was used. The following reagents were obtained from the NIH Defense and Emerging Infections Research Resources Repository, NIAID, NIH: polyclonal anti-MeV antibody, Edmonston, (antiserum, Guinea Pig), NR-4024 and polyclonal anti-CDV Lederle Avirulent (antiserum, Ferret), NR-4025.

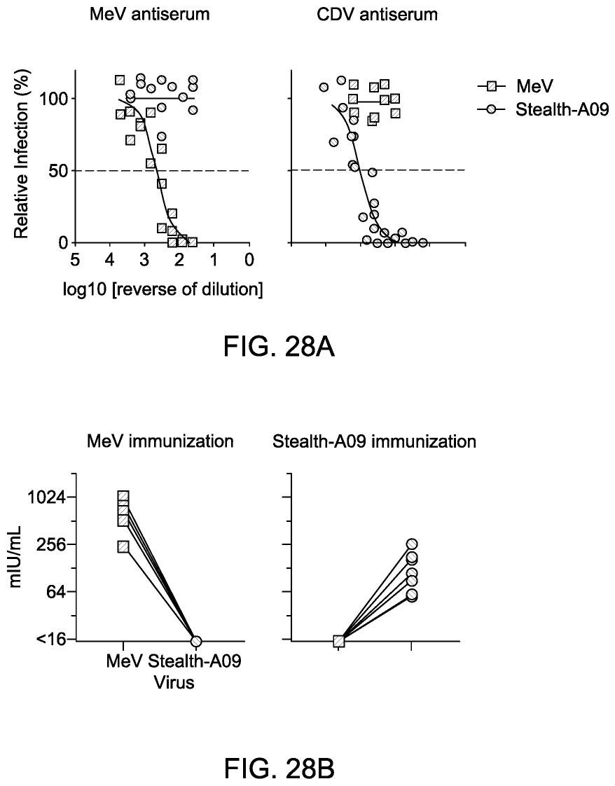

A lack of cross-neutralization between measles virus and Stealth was assessed (FIGS. 28A-B).

### Experimental Oncolytic Therapy

To establish subcutaneous tumors, 6-week-old female severe combined immunodeficiency (SCID) mice were injected in the right flank with 1×10⁷ U266.B1 tumor cells. When the tumor reached 0.5 cm in diameter, mice received a single intravenous dose of MeV (n=5) or Stealth (n=5) at 1×10 5 50% tissue culture infectious dose (TCID₅₀). Control mice (n=5) were injected with an equal volume of PBS. Animals were euthanized when the tumors ulcerated or when the burden reached 20% of the body weight. Tumor diameter was measured every other day, and tumor volume was calculated with the formula length×length×width×0.5.

To establish an orthotopic model of ovarian cancer, 5×10⁶ SKOV3ip.1 cells expressing firefly luciferase (SKOV3ip.1-Fluc) were injected into the peritoneal cavity of athymic nude mice. Ten days later, the animals received 600 mIU of measles-immune serum (Cat. #HS1017; Lot #C80553, Valley Biomedical Inc.) or an equal volume of saline, and three hours later, they were treated with a single intraperitoneal dose (1×10⁶ TCID₅₀) of MeV (n=5) or Stealth (n=5). The mice in the control group received a similar volume of Vero cell lysates (n=5). For the therapy experiment, 5×10⁶ SKOV3ip.1-Fluc cells were implanted instead. The tumor burden was monitored weekly through in vivo bioluminescence imaging using an IVIS Spectrum instrument (Perking Elmer, Waltham, MA, USA). The mice were euthanized at the end of the study (80 days), when they developed ascites or had lost 20% of their body weight. Statistical comparisons among groups were performed with the log-rank (Mantel-Cox) test, and p<0.05 was considered statistically significant.

### Statistical Analysis

Statistical analyses were performed with GraphPad Prism 8.3.1 version for Mac OS X. Significant differences among groups were determined using one-way analysis of variance (ANOVA) with Holm-Sidak's multiple comparison test Survival data were analyzed using the Kaplan-Meier method, and the log-rank test was used to identify significant differences among groups.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A CDV F polypeptide having g signal peptide sequence that is less than 75 amino acid residues in length.

2. The CDV F polypeptide of claim 1, wherein said signal peptide sequence comprises no more than 75 amino acid residues of SEQ ID NO:6.

3. The CDV F polypeptide of claim 1, wherein said CDV F polypeptide comprises SEQ ID NO:4 with the proviso that said CDV F polypeptide lacks at least amino acid residues 1 to 60 or lacks at least amino acid residues 1 to 105 of SEQ ID NO:4.

4. The CDV F polypeptide of claim 1, wherein a recombinant virus comprising said CDV F polypeptide and a CDV H polypeptide exhibits increased fusogenic activity as compared to a comparable control recombinant virus comprising a full length, wild-type CDV F polypeptide and said CDV H polypeptide.

5-7. (canceled)

8. A CDV H polypeptide comprising 454A, 464A, 479A, 494A, 510A, 520A, 525A, 526A, 527S, 528A, 529A, 537A, 539A, 547A, 548A, or a combination thereof according to the amino acid numbering of SEQ ID NO:2.

9. The CDV H polypeptide of claim 8, wherein said CDV H polypeptide comprises a combination of two, three, four, five, or six of 454A, 464A, 479A, 494A, 510A, 520A, 525A, 526A, 527S, 528A, 529A, 537A, 539A, 547A, and 548A.

10. The CDV H polypeptide of claim 8, wherein said CDV H polypeptide comprises a combination of seven, eight, nine, ten, or eleven of 454A, 464A, 479A, 494A, 510A, 520A, 525A, 526A, 527S, 528A, 529A, 537A, 539A, 547A, and 548A.

11. The CDV H polypeptide of claim 8, wherein said CDV H polypeptide comprises a combination of 12, 13, or 14 of 454A, 464A, 479A, 494A, 510A, 520A, 525A, 526A, 527S, 528A, 529A, 537A, 539A, 547A, and 548A.

12. The CDV H polypeptide of claim 8, wherein said CDV H polypeptide comprises 454A, 464A, 479A, 494A, 510A, 520A, 525A, 526A, 527S, 528A, 529A, 537A, 539A, 547A, and 548A.

13. The CDV H polypeptide of claim 8, wherein said CDV H polypeptide comprises M437 according to the amino acid numbering of SEQ ID NO:5.

14-25. (canceled)

26. A replication-competent vesicular stomatitis virus comprising an RNA molecule, wherein said RNA molecule comprises a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a CDV F polypeptide, a nucleic acid sequence that is a template for a positive sense transcript encoding a CDV H polypeptide, and a nucleic acid sequence that is a template for a positive sense transcript encoding a VSV L polypeptide, wherein said RNA molecule lacks a nucleic acid sequence that is a template for a positive sense transcript encoding a functional VSV G polypeptide.

27. The virus of claim 26, wherein said CDV F polypeptide is a CDV F polypeptide of claim 1.

28. The virus of claim 26, wherein said CDV H polypeptide is a CDV H polypeptide of claim 8.

29. The virus of claim 26, wherein said CDV H polypeptide comprises an amino acid sequence of a single chain antibody.

30. The virus of claim 29, wherein said single chain antibody is a single chain antibody directed to CD19, CD20, CD38, CD46, EGFR, αFR, HER2/neu, or PSMA.

31. The virus of claim 26, wherein said RNA molecule comprises a nucleic acid sequence that is a template for a positive sense transcript encoding a NIS polypeptide.

32-39. (canceled)

40. A method for treating cancer, wherein said method comprises administering a composition comprising said replication-competent vesicular stomatitis virus of claim 26 to a mammal comprising cancer cells, wherein the number of cancer cells within said mammal is reduced following said administration.

41. The method of claim 40, wherein said mammal is a human.

42. The method of claim 40, wherein said cancer is myeloma, melanoma, glioma, lymphoma, mesothelioma, lung cancer, brain cancer, stomach cancer, colon cancer, rectum cancer, kidney cancer, prostate cancer, ovary cancer, breast cancer, pancreas cancer, liver cancer, or head and neck cancer.

43-46. (canceled)
